(19) United States
(12) Patent Application Publication
(10) Pub. No.: US 2025/0092054 A1
(43) Pub. Date: Mar. 20, 2025

(54) FUSED PYRIDINES FOR CONTROLLING INVERTEBRATE PESTS

(71) Applicant: FMC Corporation, Philadelphia, PA (US)

(72) Inventors: Ming XU, Middletown, DE (US); George Philip LAHM, Wilmington, DE (US); Andrew Jon DEANGELIS, Garnet Valley, PA (US)

(21) Appl. No.: 18/566,378

(22) PCT Filed: May 31, 2022
(86) PCT No.: PCT/US22/31512

§ 371 Date: Dec. 1, 2023

Related U.S. Application Data
(60) Provisional application No. 63/195,965, filed on Jun. 2, 2021.

(51) Int. Cl. C07D 491/048; A01N 43/90; A01P 7/04; C07D 495/04

(52) U.S. Cl. CPC C07D 491/048; A01N 43/90; A01P 7/04; C07D 495/04

(57) ABSTRACT

Disclosed are compounds of Formula 1, including all geometric and stereoisomers,

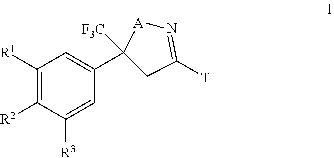

wherein
R¹, R², R³, A and T are as defined in the disclosure.
   Also disclosed are compositions containing the compounds of Formula 1 and methods for controlling an invertebrate pest comprising contacting the invertebrate pest or its environment with a biologically effective amount of a compound or a composition of the disclosure.

### CROSS-REFERENCE TO RELATED APPLICATION

This application claims the benefit of U.S. Provisional Application No. 63/195,965, filed Jun. 2, 2021, all of which is incorporated by reference herein in its entirety.

### FIELD

This disclosure relates to certain fused pyridines, their N-oxides, salts and compositions suitable for agronomic and nonagronomic uses, and methods of their use for controlling invertebrate pests such as arthropods in both agronomic and nonagronomic environments.

### BACKGROUND

The control of invertebrate pests is extremely important in achieving high crop efficiency. Damage by invertebrate pests to growing and stored agronomic crops can cause significant reduction in productivity and thereby result in increased costs to the consumer.

The control of invertebrate pests in forestry, greenhouse crops, ornamentals, nursery crops, stored food and fiber products, livestock, household, turf, wood products, and public and animal health is also important. Many products are commercially available for these purposes, but the need continues for new compounds that are more effective, less costly, less toxic, environmentally safer or have different sites of action.

### SUMMARY

This disclosure is directed to compounds of Formula 1 (including all geometric and stereoisomers), N-oxides, and salts thereof, and compositions containing them and their use for controlling invertebrate pests:

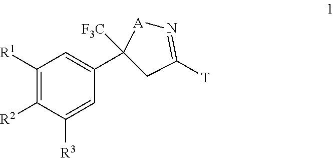

wherein
T is

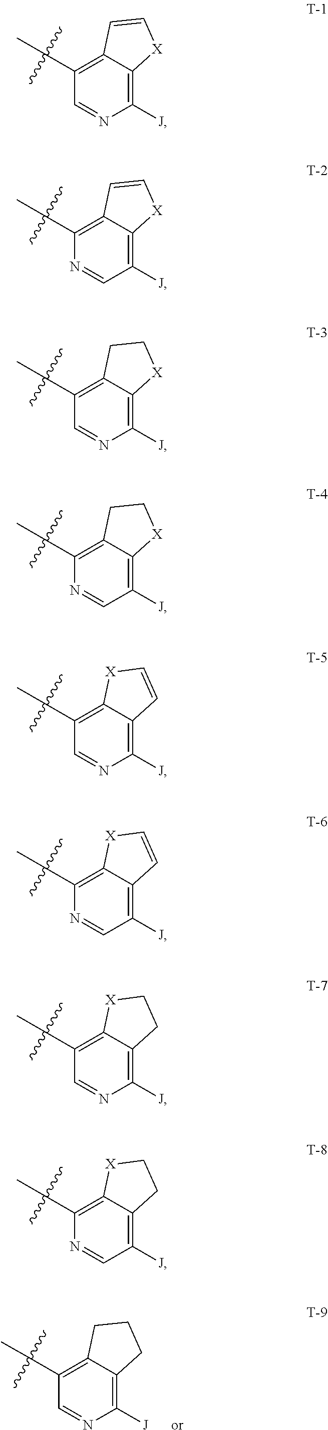

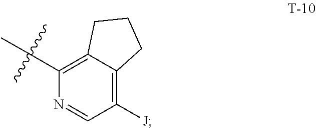

A is O, S or CH₂;
R¹, R², and R³ are each independently hydrogen, halogen, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
X is O or S;
J is C(═Z)NR⁴R⁵ or CH(R⁶)N(R¹³)C(═Z)R¹⁴;
each Z is independently O or S;
R⁴ is hydrogen, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl or C₂-C₇ alkoxycarbonyl;
R⁵ is hydrogen, OR¹⁰, NR¹¹R¹², SO₂NR¹¹R¹², C(R¹²)═NOR¹¹, CHR¹²NHR¹¹ or Q¹; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷; or
R⁴ and R⁵ are taken together with the nitrogen to which they are attached to form a 3- to 6-membered ring containing ring members selected from carbon atoms and up to two additional atoms independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
R⁶ is hydrogen, halogen, cyano, C₁-C₃ alkyl or C₁-C₃ haloalkyl;
each R⁷ is independently halogen, hydroxy, amino, cyano, nitro, Q², C₁-C₆ alkyl, C₁-C₆ alkoxy, C₁-C₆ alkylthio, C₁-C₆ alkylsulfinyl, C₁-C₆ alkylsulfonyl, C₁-C₆ alkylamino, C₂-C₈ dialkylamino, C₃-C₆ cycloalkylamino, C₂-C₇ alkylcarbonyl, C₂-C₇ alkoxycarbonyl, C₂-C₇ alkylaminocarbonyl, C₃-C₇ cycloalkylaminocarbonyl, C₃-C₇ alkenylaminocarbonyl, C₃-C₇ alkynylaminocarbonyl, C₃-C₉ dialkylaminocarbonyl, C₂-C₇ haloalkylcarbonyl, C₂-C₇ haloalkoxycarbonyl, C₂-C₇ haloalkylaminocarbonyl, C₃-C₉ halodialkylaminocarbonyl or —CONH₂;
Q¹ is a 5- or 6-membered aromatic ring or a 4- to 11-membered saturated or unsaturated ring or ring system, each optionally containing up to three heteroatoms selected from up to 1 oxygen, up to 1 sulfur and up to 3 nitrogen, wherein up to 2 carbon atom ring members are independently selected from C(═O) and C(═S) and the sulfur atom ring member is selected from S, S(═O) and S(═O)₂, each ring or ring system optionally substituted with one or more substituents independently selected from R⁸;
each Q² is independently a phenyl ring, a 5- or 6-membered aromatic heterocyclic ring or a 3- to 6-membered non-aromatic heterocyclic ring, each ring optionally substituted with one or more substituents independently selected from R⁹;
each R⁸ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, C₂-C₇ alkylaminocarbonyl, C₃-C₉ dialkylaminocarbonyl, —CONH₂, cyano or nitro;
each R⁹ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, cyano, nitro, phenyl or pyridinyl;
R¹⁰ is hydrogen; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more halogen;
R¹¹ is hydrogen, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl, C₂-C₇ haloalkylcarbonyl or C₂-C₇ alkoxycarbonyl;
R¹² is hydrogen or Q³; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷; or
R¹¹ and R¹² are taken together with the nitrogen to which they are attached to form a 3- to 6-membered ring containing ring members selected from carbon atoms and up to one additional atom independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
Q³ is a phenyl ring or a 5- or 6-membered heterocyclic ring, each ring optionally substituted with one or more substituents independently selected from R⁹;
R¹³ is hydrogen, C₁-C₆ alkyl, C₂-C₇ alkylcarbonyl, C₂-C₄ haloalkylcarbonyl, C₂-C₇ alkoxycarbonyl or C₂-C₄ alkoxyalkyl;
R¹⁴ is C₁-C₆ alkyl optionally substituted with halogen, OR¹⁹, S(═O)ₙR²⁰ or NR²¹C(═O)R²²; or
R¹⁴ is C₃-C₆ cycloalkyl or C₄-C₇ cycloalkylalkyl, each optionally substituted with up to one cyclopropyl and up to 4 substituents selected from halogen, cyano, C₁-C₂ alkyl and C₁-C₂ haloalkyl; or
R¹⁴ is (CH₂)ₘQ⁴; or
R¹⁴ is OR¹⁶ or NR^{17a}R^{17b};
Q⁴ is a 3- to 6-membered saturated heterocyclic ring containing ring members selected from carbon atoms and one heteroatom independently selected from one oxygen and one sulfur, wherein the sulfur atom ring member is selected from S, S(═O) or S(═O)₂, each ring optionally substituted with up to 2 substituents independently selected from R¹⁸;
R¹⁶ is C₁-C₄ alkyl or C₁-C₄ haloalkyl;
R^{17a} is hydrogen, C₁-C₄ alkyl, C₁-C₄ haloalkyl or C₃-C₆ cycloalkyl; R^{17b} is hydrogen, C₁-C₄ alkyl, C₁-C₄ haloalkyl or C₃-C₆ cycloalkyl; or
R^{17a} and R^{17b} are taken together with the nitrogen to which they are attached to form a 3- to 6-membered ring containing ring members selected from carbon atoms and up to one additional atom independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
each R¹⁸ is independently halogen, cyano, C₁-C₂ alkyl or C₁-C₂ haloalkyl;
R¹⁹ is hydrogen, C₁-C₄ alkyl or C₁-C₄ haloalkyl;
R²⁰ is C₁-C₄ alkyl or C₁-C₄ haloalkyl;
R²¹ is hydrogen or C₁-C₄ alkyl;
R²² is C₁-C₄ alkyl, C₁-C₄ haloalkyl or C₃-C₆ cycloalkyl;
n is independently 0, 1 or 2; and
m is 0 or 1.

This disclosure also provides a composition comprising a compound of Formula 1, an N-oxide or a salt thereof, and at least one additional component selected from the group consisting of surfactants, solid diluents and liquid diluents. In one embodiment, this disclosure also provides a composition for controlling an invertebrate pest comprising a compound of Formula 1, an N-oxide or a salt thereof, and at least one additional component selected from the group consisting of surfactants, solid diluents and liquid diluents, said composition optionally further comprising at least one additional biologically active compound or agent.

This disclosure also provides a method for controlling an invertebrate pest comprising contacting the invertebrate pest or its environment with a biologically effective amount of a compound of Formula 1, an N-oxide or a salt thereof, (e.g., as a composition described herein). This disclosure also relates to such method wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent.

This disclosure also provides a method for controlling an invertebrate pest comprising contacting the invertebrate pest or its environment with a biologically effective amount of any of the aforesaid compositions wherein the environment is a plant.

This disclosure also provides a method for controlling an invertebrate pest comprising contacting the invertebrate pest or its environment with a biologically effective amount of any of the aforesaid compositions wherein the environment is an animal.

This disclosure also provides a method for controlling an invertebrate pest comprising contacting the invertebrate pest or its environment with a biologically effective amount of any of the aforesaid compositions wherein the environment is a seed.

This disclosure also provides a method for protecting a seed from an invertebrate pest comprising contacting the seed with a biologically effective amount of a compound of Formula 1, an N-oxide or a salt thereof, (e.g., as a composition described herein). This disclosure also relates to the treated seed (i.e. seed contacted with a compound of Formula 1).

This disclosure also provides a method for increasing vigor of a crop plant comprising contacting the crop plant, the seed from which the crop plant is grown or the locus (e.g., growth medium) of the crop plant with a biologically effective amount of a compound of Formula 1 (e.g., as a composition described herein).

This disclosure further provides a method for protecting an animal from an invertebrate parasitic pest comprising administering to the animal a parasiticidally effective amount of a compound of Formula 1, an N-oxide or a salt thereof, (e.g., as a composition described herein). This disclosure also provides for the use of a compound of Formula 1, an N-oxide or a salt thereof, (e.g., as a composition described herein) in protecting an animal from an invertebrate pest.

### DETAILED DESCRIPTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed disclosure. The term “consisting essentially of” occupies a middle ground between “comprising” and “consisting of”.

Where applicants have defined an embodiment or a portion thereof with an open-ended term such as “comprising,” it should be readily understood that (unless otherwise stated) the description should be interpreted to also describe such an embodiment using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the disclosure are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods, nematodes and helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes members of the phylum Nematoda, such as phytophagous nematodes and helminth nematodes parasitizing animals. The term “helminth” includes all of the parasitic worms, such as roundworms (phylum Nematoda), heartworms (phylum Nematoda, class Secernentea), flukes (phylum Platyhelminthes, class Tematoda), acanthocephalans (phylum Acanthocephala), and tapeworms (phylum Platyhelminthes, class Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) an invertebrate pest to be controlled or its environment, or to a plant, the seed from which the plant is grown, or the locus of the plant (e.g., growth medium) to protect the plant from injury by the invertebrate pest or for other desired effect (e.g., increasing plant vigor).

Nonagronomic applications include protecting an animal from an invertebrate parasitic pest by administering a parasiticidally effective (i.e. biologically effective) amount of a compound of the disclosure, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

A wavy line in a structure fragment denotes the attachment point of the fragment to the remainder of the molecule. For example, when the variable T in Formula 1 is defined as T-1, the wavy line bisecting the bond in the position identified with an asterisk (*) of the fused pyridine T-1 means that the fused pyridine T-1 is attached to the remainder of the structure of Formula 1 at said position, as shown below.

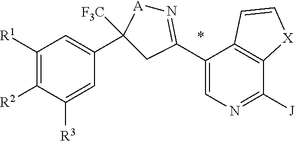

A “-” at the beginning of a fragment definition denotes the attachment point of said fragment to the remainder of the molecule; for example, “—CH₂CH₂OMe” denotes the fragment 2-methoxyethyl.

Cyclic fragments are represented by the use of two “-” within parentheses; for example, the fragment 1-cyanocyclopropyl is represented by “—C(CN)(—CH₂CH₂—)”, wherein a carbon atom is bonded to both terminal carbon atoms of the two-carbon chain, as illustrated below.

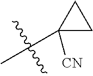 [SMILES: *C1(C#N)CC1]

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkylamino” includes an NH radical substituted with straight-chain or branched alkyl. Examples of “alkylamino” include CH₃CH₂NH, CH₃CH₂CH₂NH, (CH₃)₂CHNH and the different butylamino, pentylamino and hexylamino isomers. Examples of “dialkylamino” include (CH₃)₂N, (CH₃CH₂)₂N, CH₃CH₂(CH₃)N and (CH₃)₂CHCH₂CH₂(CH₃CH₂)N. “Alkenylamine” includes an NH radical substituted with straight-chain or branched alkenes. Examples of “Alkenylamine” include CH₂═CHNH, CH₃CH═C(CH₃)CH₂NH, (CH₃)₂CHCH═CHNH and the different butenylamine, pentenylamine and hexenylamine isomers. “Alkynylamine” includes an NH radical substituted with straight-chain or branched alkynes. Examples of “Alkynylamine” include HC≡CNH, CH₃C≡CCH₂NH, (CH₃)₂CHC≡CNH and the different butynylamine, pentynylamine and hexynylamine isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

“Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers.

“Alkylcarbonyl” denotes a straight-chain or branched alkyl group bonded to a C(═O) moiety. Examples of “alkylcarbonyl” include CH₃C(═O), CH₃CH₂CH₂C(═O), (CH₃)₂CHC(═O) and the different butylcarbonyl, pentylcarbonyl and hexylcarbonyl isomers. Examples of “alkoxycarbonyl” include CH₃OC(═O), CH₃CH₂OC(═O), CH₃CH₂CH₂OC(═O), (CH₃)₂CHOC(═O), and the different butoxycarbonyl, pentylcarbonyl and hexylcarbonyl isomers. Examples of “alkylaminocarbonyl” include CH₃NHC(═O), CH₃CH₂NHC(═O), CH₃CH₂CH₂NHC(═O), (CH₃)₂CHNHC(═O), and the different butylaminocarbonyl, pentylaminocarbonyl and hexylaminocarbonyl isomers. Examples of “dialkylaminocarbonyl” include (CH₃)₂NC(═O), (CH₃CH₂)₂NC(═O), CH₃CH₂(CH₃)NC(═O), (CH₃CH₂)₂CH(CH₃)NC(═O) and CH₃CH₂CH₂(CH₃CH₂)NC(═O).

The term “alkenylaminocarbonyl” denotes a straight-chain or branched alkenylamino attached to and linked through a C(═O) group. Examples of “alkenylaminocarbonyl” include CH₂═CHNHC(═O), CH₃CH═C(CH₃)CH₂NHC(═O) and (CH₃)₂CHCH═CHNHC(═O). The term “alkynylaminocarbonyl” denotes a straight-chain or branched alkynylamino attached to and linked through a C(═O) group. Examples of “alkynylaminocarbonyl” include HC≡CNHC(═O), CH₃CH≡CCH₂NHC(═O) and (CH₃)₂CHC≡CNHC(═O).

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkylamino” denotes an NH radical substituted with cycloalkyl. Examples of “cycloalkylamino” include cyclopropylamino and cyclohexylamino. “Cycloalkylaminocarbonyl” denotes cycloalkylamino bonded to a C(═O) group, for example, cyclopentylaminocarbonyl and cyclohexylaminocarbonyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkylcarbonyl”, “haloalkoxycarbonyl”, “haloalkylaminocarbonyl”, “halodialkylaminocarbonyl” and the like, are defined analogously to the term “haloalkyl”. Examples of “halocycloalkyl” include chlorocyclopropyl, fluorocyclobutyl and chlorocyclohexyl. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

The chemical abbreviations S(O) and S(═O) as used herein represent a sulfinyl moiety.

The chemical abbreviations SO₂, S(O)₂ and S(═O)₂ as used herein represent a sulfonyl moiety. The chemical abbreviations C(O) and C(═O) as used herein represent a carbonyl moiety. The chemical abbreviations CO₂, C(O)O and C(═O)O as used herein represent an oxycarbonyl moiety. “CHO” means formyl.

The total number of carbon atoms in a substituent group is indicated by the “Cₕ-Cᵢ” prefix where h and i are numbers from 1 to 9. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including (CH₃)₂CHOCH₂—, CH₃CH₂CH₂OCH₂— and CH₃CH₂OCH₂CH₂—.

When a compound is substituted with a substituent bearing a subscript that indicates the number of said substituents can vary (e.g., (R^{V})ᵣ in Exhibit 1 wherein r is 1, 2, 3 or 4), then said substituents are independently selected from the group of defined substituents, unless otherwise indicated. Further, when the subscript indicates a range, e.g. (R)ₕ₋ᵢ, then the number of substituents may be selected from the integers between h and i inclusive. When a group contains a substituent which can be hydrogen, for example R¹, then when this substituent is taken as hydrogen, it is recognized that this is equivalent to said group being unsubstituted. When a variable group is shown to be optionally attached to a position, for example (R^{V})ᵣ in Exhibit 1 wherein r may be 0, then hydrogen may be at the position even if not recited in the definition of the variable group. When one or more positions on a group are said to be “not substituted” or “unsubstituted”, then hydrogen atoms are attached to take up any free valency.

The number of optional substituents may be restricted by an expressed limitation. For example, the phrase “optionally substituted with up to 2 substituents independently selected from R¹⁸” means that 0, 1 or 2 substituents can be present (if the number of potential connection points allows). When a range specified for the number of substituents (e.g., r being an integer from 0 to 4 in Exhibit 1) exceeds the number of positions available for substituents on a ring (e.g., in Exhibit 1 only 2 positions are available for (R^{V})ᵣ on U-9), the actual higher end of the range is recognized to be the number of available positions.

Naming of substituents in the present disclosure uses recognized terminology providing conciseness in precisely conveying to those skilled in the art the chemical structure. For sake of conciseness, locant descriptors may be omitted.

Unless otherwise indicated, a “ring” or “ring system” as a component of Formula 1 (e.g., substituent Q¹) is carbocyclic or heterocyclic. The term “ring system” denotes two or more connected rings. The term “bicyclic ring system” denotes a ring system consisting of two rings sharing one or more common atoms. A “bicyclic ring system” can be “ortho-fused”, “bridged bicyclic” or “spirobicyclic”. In an “ortho-fused bicyclic ring system” the common atoms are adjacent, and therefore the rings share two adjacent atoms and a bond connecting them. A “bridged bicyclic ring system” is formed by bonding a segment of one or more atoms to nonadjacent ring members of a ring. A “spirobicyclic ring system” is formed by bonding a segment of two or more atoms to the same ring member of a ring. The term “fused heterobicyclic ring system” denotes a fused bicyclic ring system in which at least one ring atom is not carbon. The term “ring member” refers to an atom (e.g., C, O, N, B or S) or other moiety (e.g., C(═O), C(═S), S(═O) or S(═O)₂) forming the backbone of a ring or ring system.

The terms “carbocyclic ring”, “carbocycle” or “carbocyclic ring system” denote a ring or ring system wherein the atoms forming the ring backbone are selected only from carbon. The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a carbocyclic ring or heterocyclic ring can be a saturated or unsaturated ring. “Saturated” refers to a ring having a backbone consisting of atoms linked to one another by single bonds; unless otherwise specified, the remaining atom valences are occupied by hydrogen atoms. Unless otherwise stated, an “unsaturated ring” may be partially unsaturated or fully unsaturated. The expression “fully unsaturated ring” means a ring of atoms in which the bonds between atoms in the ring are single or double bonds according to valence bond theory and furthermore the bonds between atoms in the ring include as many double bonds as possible without double bonds being cumulative (i.e. no C═C═C or C═C═N). The term “partially unsaturated ring” denotes a ring comprising at least one ring member bonded to an adjacent ring member through a double bond and which conceptually potentially accommodates a number of non-cumulated double bonds between adjacent ring members (i.e. in its fully unsaturated counterpart form) greater than the number of double bonds present (i.e. in its partially unsaturated form).

Unless otherwise indicated, heterocyclic rings and ring systems can be attached through any available carbon or nitrogen by replacement of a hydrogen on said carbon or nitrogen.

The term “aromatic” indicates that each of the ring atoms of a fully unsaturated ring are essentially in the same plane and have a p-orbital perpendicular to the ring plane, and that (4n+2)π electrons, where n is a positive integer, are associated with the ring to comply with Hückel's rule.

The term “aromatic ring system” denotes a carbocyclic or heterocyclic ring system in which at least one ring of the ring system is aromatic. When a fully unsaturated carbocyclic ring satisfies Hückel's rule, then said ring is also called an “aromatic ring” or “aromatic carbocyclic ring”. The term “aromatic carbocyclic ring system” denotes a carbocyclic ring system in which at least one ring of the ring system is aromatic. When a fully unsaturated heterocyclic ring satisfies Hückel's rule, then said ring is also called a “heteroaromatic ring” or “aromatic heterocyclic ring”. The term “aromatic heterocyclic ring system” denotes a heterocyclic ring system in which at least one ring of the ring system is aromatic.

The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” denotes a carbocyclic ring in which no ring in the ring system is aromatic. The term “nonaromatic heterocyclic ring system” denotes a heterocyclic ring system in which no ring in the ring system is aromatic.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When Q¹ is a 5- or 6-membered nitrogen-containing heterocyclic ring, it may be attached to the remainder of Formula 1 though any available carbon or nitrogen ring atom, unless otherwise described. As noted above, Q¹ can be (among others) phenyl optionally substituted with one or more substituents selected from a group of substituents as defined in the Summary. An example of phenyl optionally substituted with one to five substituents is the ring illustrated as U-1 in Exhibit 1, wherein R^{V} is R⁸ as defined in the Summary for Q¹ and r is an integer from 0 to 5.

As noted above, Q¹ can be (among others) 5- or 6-membered heterocyclic ring, which may be saturated or unsaturated, optionally substituted with one or more substituents selected from a group of substituents as defined in the Summary. Examples of a 5- or 6-membered unsaturated aromatic heterocyclic ring optionally substituted with from one or more substituents include the rings U-2 through U-61 illustrated in Exhibit 1 wherein R^{V} is any substituent as defined in the Summary for Q¹ (i.e. R⁸) and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{V})ᵣ.

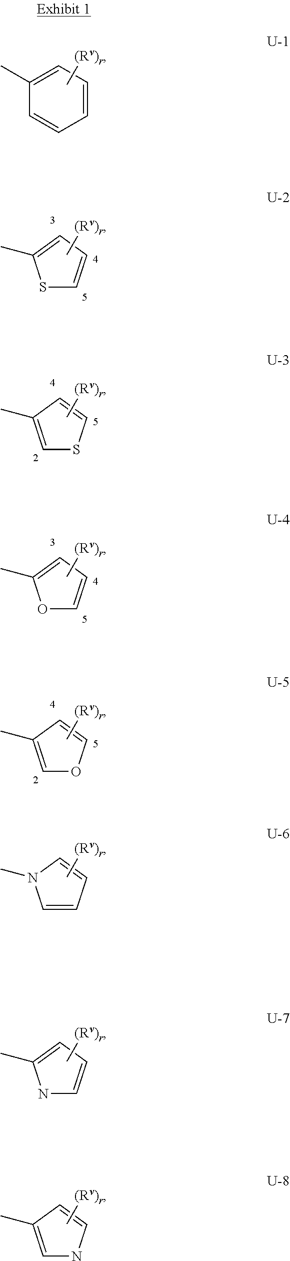

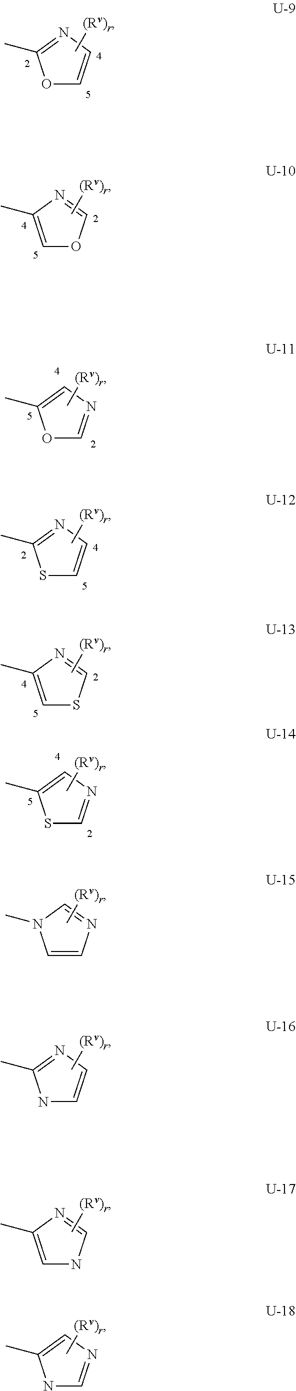

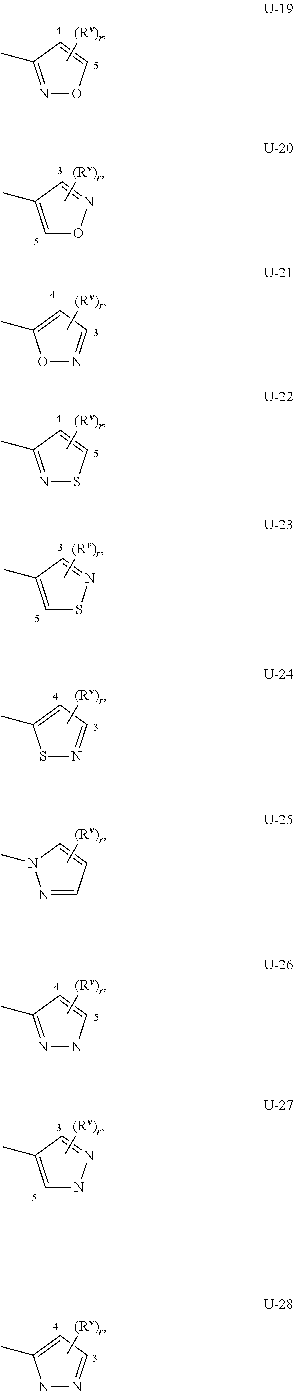

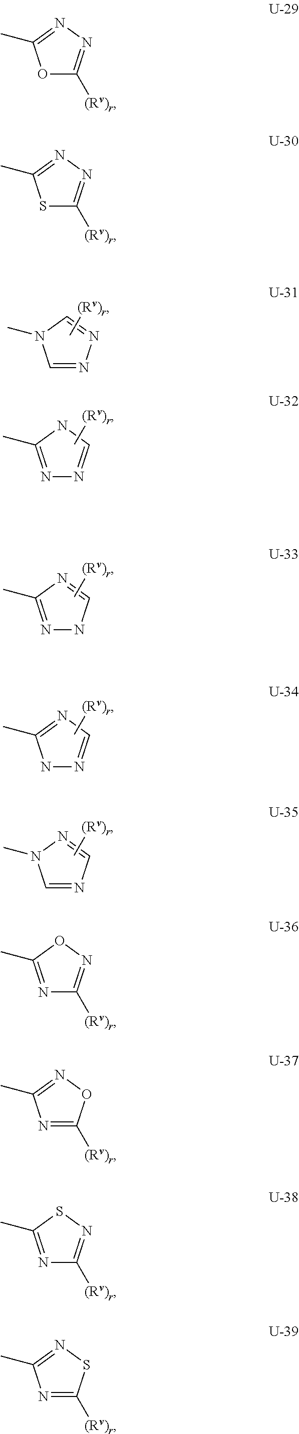

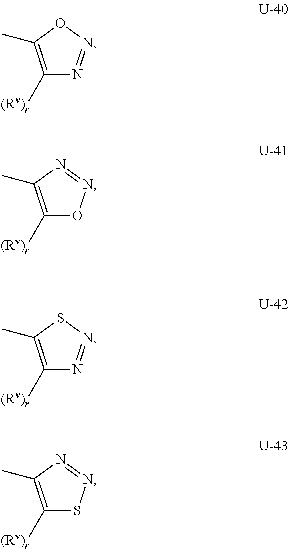

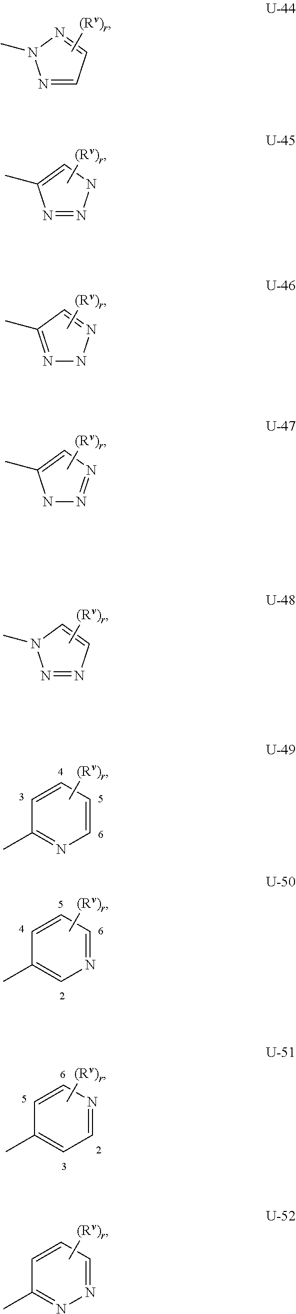

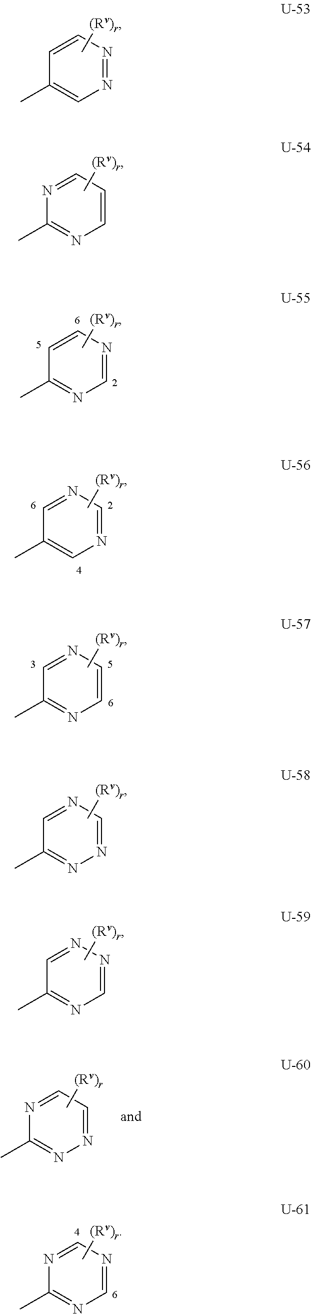

Note that when Q¹ is a 5- or 6-membered saturated or unsaturated non-aromatic heterocyclic ring optionally substituted with one or more substituents selected from the group of substituents as defined in the Summary for Q¹, one or two carbon ring members of the heterocycle can optionally be in the oxidized form of a carbonyl moiety.

Examples of a 5- or 6-membered saturated or non-aromatic unsaturated heterocyclic ring include the rings G-1 through G-35 as illustrated in Exhibit 2. Note that when the attachment point on the G group is illustrated as floating, the G group can be attached to the remainder of Formula 1 through any available carbon or nitrogen of the G group by replacement of a hydrogen atom. The optional substituents corresponding to R^{V} can be attached to any available carbon or nitrogen by replacing a hydrogen atom. For these G rings, r is typically an integer from 0 to 4, limited by the number of available positions on each G group.

Note that when Q¹ comprises a ring selected from G-28 through G-35, G² is selected from O, S or N. Note that when G² is N, the nitrogen atom can complete its valence by substitution with either H or the substituents corresponding to R^{V} as defined in the Summary for Q¹, Q² or Q³ (i.e. R⁸ or R⁹).

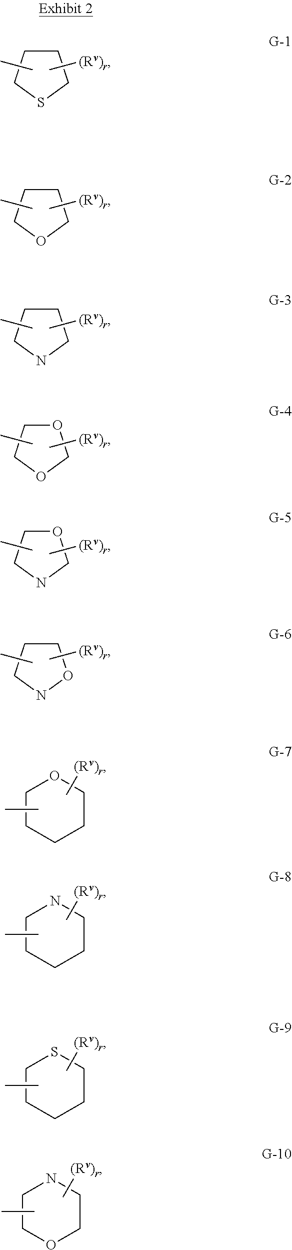

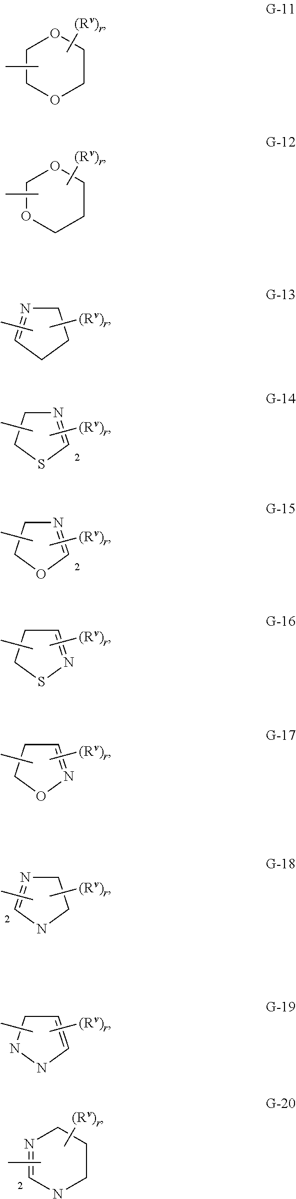

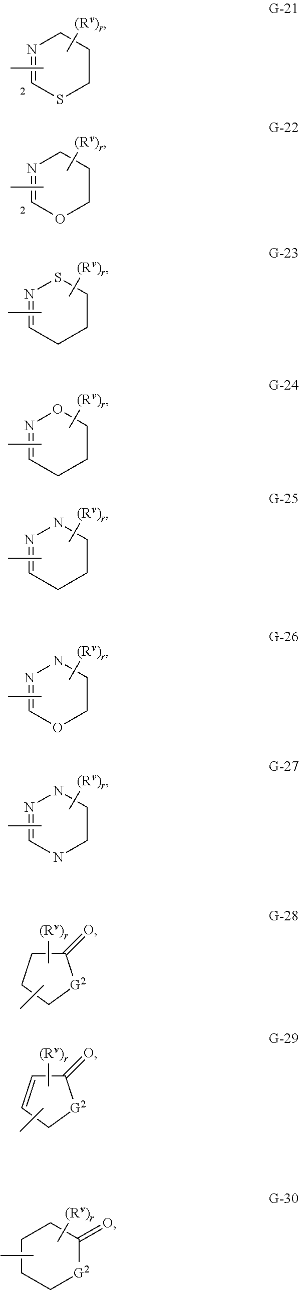

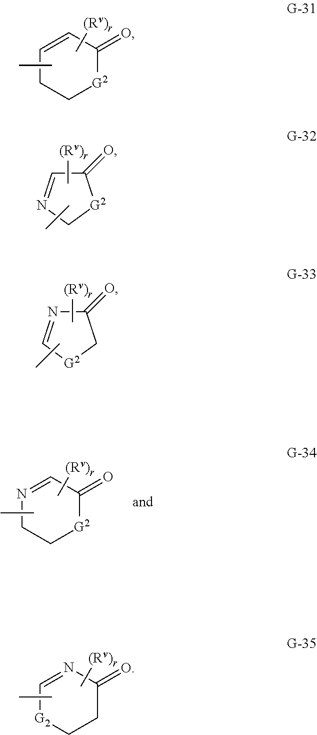

As noted above, Q¹ can be (among others) an 8-, 9- or 10-membered ortho-fused bicyclic ring system optionally substituted with one or more substituents selected from a group of substituents as defined in the Summary (i.e. R⁸). Examples of 8-, 9- or 10-membered ortho-fused bicyclic ring system optionally substituted with from one or more substituents include the rings U-81 through U-123 illustrated in Exhibit 3 wherein R^{V} is any substituent as defined in the Summary for Q¹ (i.e. R⁸), and r is typically an integer from 0 to 4.

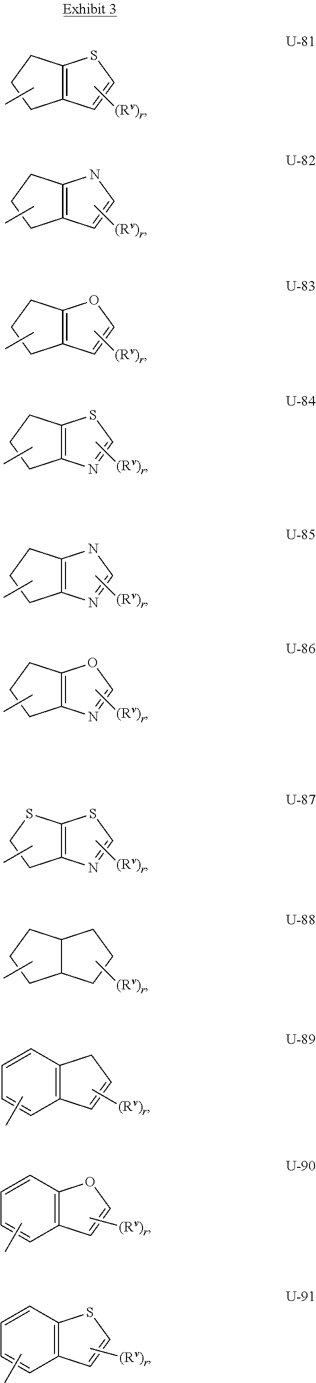

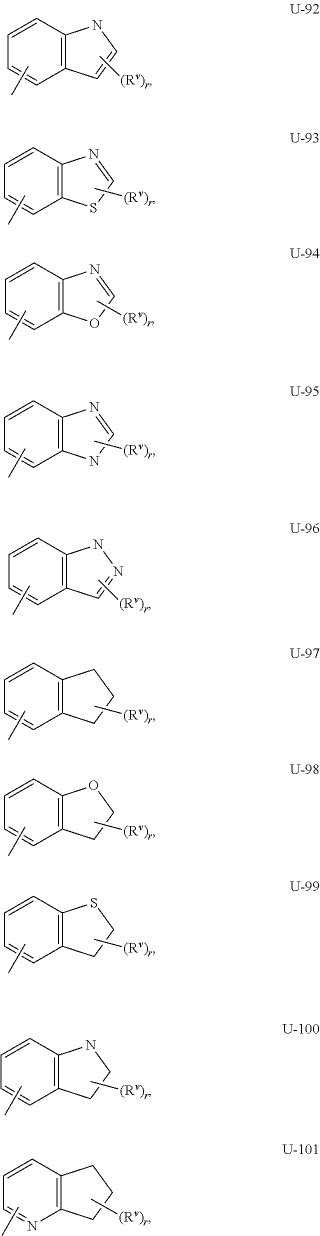

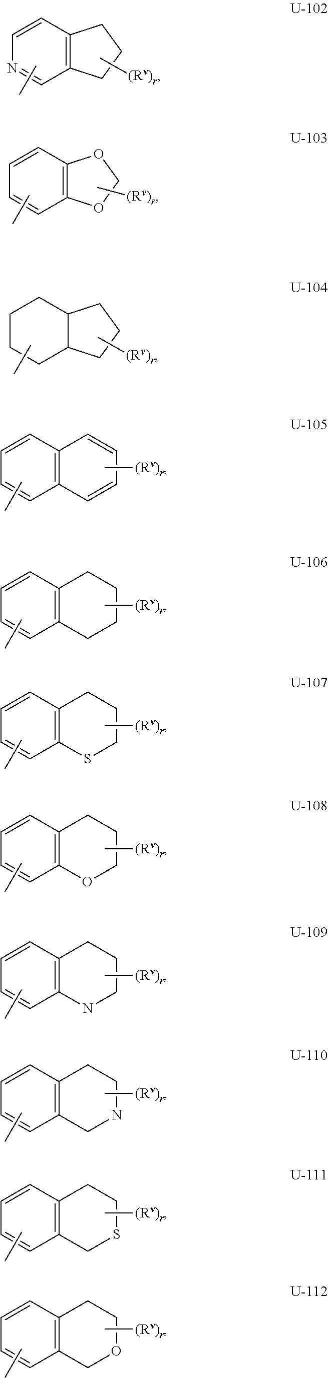

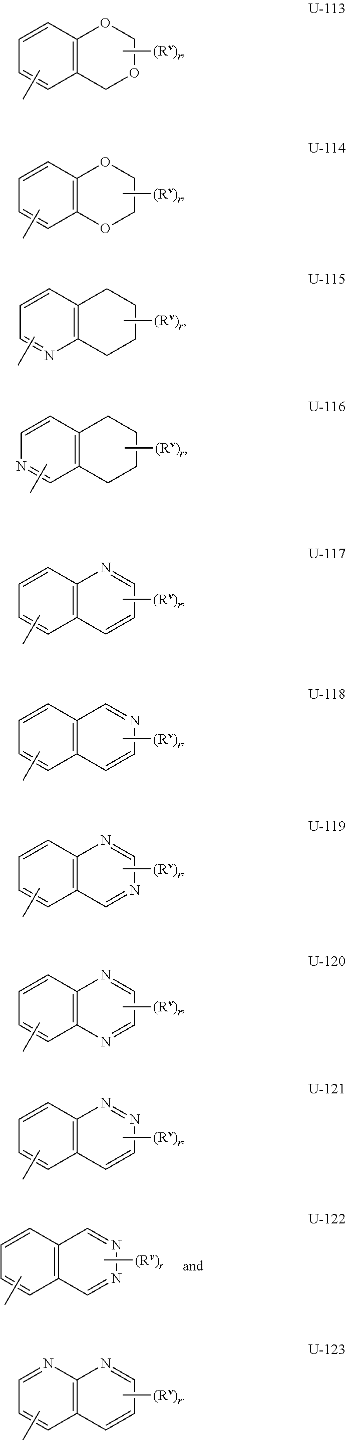

Although R^{V} groups are shown in the structures U-1 through U-123, it is noted that they do not need to be present since they are optional substituents. Note that when R^{V} is H when attached to an atom, this is the same as if said atom is unsubstituted. The nitrogen atoms that require substitution to fill their valence are substituted with H or R^{V}. Note that when the attachment point between (R^{V})ᵣ and the U group is illustrated as floating, (R^{V})ᵣ can be attached to any available carbon atom or nitrogen atom of the U group. Note that when the attachment point on the U group is illustrated as floating, the U group can be attached to the remainder of Formula 1 through any available carbon or nitrogen of the U group by replacement of a hydrogen atom. Note that some U groups can only be substituted with less than 4 R^{V} groups (e.g., U-2 through U-5, U-7 through U-48, and U-52 through U-61).

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this disclosure can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

The compounds of the disclosure may be present as a mixture of stereoisomers or individual stereoisomers. For example, two possible enantiomers of Formula 1 are depicted as Formula 1′ and Formula 1″ involving the isoxazoline chiral center identified with an asterisk (*). Analogously, other chiral centers are possible at, for example, R⁵.

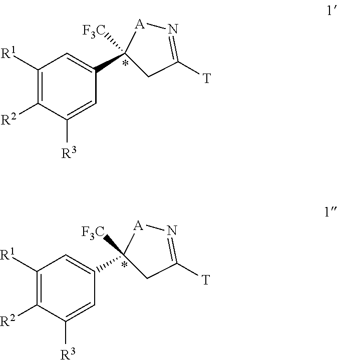

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the broad end of the wedge is attached to the atom further away from the viewer.

The compounds of the disclosure can exist as stereoisomers due to the possible chiral carbon atoms present in Formula 1. Thus, this disclosure comprises the individual stereoisomers of the compounds of Formula 1, as well as mixtures of stereoisomers of the compounds of Formula 1.

This disclosure comprises racemic mixtures, for example, equal amounts of the enantiomers of Formulae 1′ and 1″. In addition, this disclosure includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x-1) 100%, where x is the mole fraction of the dominant enantiomer in the mixture (e.g., an ee of 20% corresponds to a 60:40 ratio of enantiomers). Preferably the compositions of this disclosure have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R⁵ may themselves contain chiral centers. This disclosure comprises racemic mixtures as well as enriched and essentially pure stereoconfigurations at these additional chiral centers.

Compounds of this disclosure can exist as one or more conformational isomers due to restricted rotation about the amide bond (e.g., C(═O)—N) in Formula 1. This disclosure comprises mixtures of conformational isomers. In addition, this disclosure includes compounds that are enriched in one conformer relative to others.

This disclosure comprises all stereoisomers, conformational isomers and mixtures thereof in all proportions as well as isotopic forms such as deuterated compounds.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides.

Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of invertebrate pests. The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present disclosure comprises compounds selected from Formula 1, N-oxides and suitable salts thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. Compounds of this disclosure may exist as one or more crystalline polymorphs. This disclosure comprises both individual polymorphs and mixtures of polymorphs, including mixtures enriched in one polymorph relative to others. For a comprehensive discussion of polymorphism see R. Hilfiker, Ed., Polymorphism in the Pharmaceutical Industry, Wiley-VCH, Weinheim, 2006.

Embodiments of the present disclosure as described in the Summary include those described below. In the following Embodiments, Formula 1 includes stereoisomers, N-oxides and salts thereof, and reference to “a compound of Formula 1” includes the definitions of substituents specified in the Summary unless further defined in the Embodiments.
Embodiment 1. A compound of Formula 1 wherein T is T-1, T-2, T-3, T-4, T-5, T-6, T-7 or T-8.
Embodiment 2. A compound of Embodiment 1 wherein T is T-1, T-2, T-4, T-5 or T-6.
Embodiment 3. A compound of Formula 1 or any of the preceeding Embodiments wherein T is T-1, T-2, T-5 or T-6.
Embodiment 4. A compound of Embodiment 3 wherein T is T-1, T-2 or T-6.
Embodiment 5. A compound of Embodiment 3 wherein T is T-1 or T-2.
Embodiment 6. A compound of Embodiment 3 wherein T is T-1 or T-5.
Embodiment 7. A compound of Embodiment 3 wherein T is T-2 or T-6.
Embodiment 8. A compound of Embodiment 3 wherein T is T-5 or T-6.
Embodiment 9. A compound of Embodiment 3 wherein T is T-1.
Embodiment 10. A compound of Embodiment 3 wherein T is T-2.
Embodiment 11. A compound of Embodiment 3 wherein T is T-5.
Embodiment 12. A compound of Embodiment 3 wherein T is T-6.
Embodiment 13. A compound of Formula 1 wherein T is T-3, T-4, T-7, T-8, T-9 or T-10.
Embodiment 14. A compound of Formula 1 or Embodiment 13 wherein T is T-3, T-4, T-7 or T-8.
Embodiment 15. A compound of Embodiment 14 wherein T is T-3.
Embodiment 16. A compound of Embodiment 14 wherein T is T-4.
Embodiment 17. A compound of Embodiment 14 wherein T is T-7.
Embodiment 18. A compound of Embodiment 14 wherein T is T-8.
Embodiment 19. A compound of Formula 1 or Embodiment 13 wherein T is T-9 or T-10.
Embodiment 20. A compound of Embodiment 19 wherein T is T-9.
Embodiment 21. A compound of Embodiment 19 wherein T is T-10.
Embodiment 22. A compound of Formula 1 or any one of Embodiments 1 through 21 wherein A is O or CH₂.
Embodiment 23. A compound of Formula 1 or any one of Embodiments 1 through 21 wherein A is O or S.
Embodiment 24. A compound of Embodiment 22 or Embodiment 23 wherein A is O.
Embodiment 25. A compound of Embodiment 22 wherein A is CH₂.
Embodiment 26. A compound of Embodiment 23 wherein A is S.
Embodiment 27. A compound of Formula 1 or any of the preceding Embodiments wherein R¹, R², and R³ are each independently hydrogen, halogen, cyano, C₁-C₄ alkyl, C₁-C₄ haloalkyl, C₁-C₄ alkoxy or C₁-C₄ haloalkoxy.
Embodiment 28. A compound of Embodiment 27 wherein R¹, R², and R³ are each independently hydrogen, halogen, C₁-C₂ alkyl, C₁-C₂ haloalkyl, C₁-C₂ alkoxy or C₁-C₂ haloalkoxy.
Embodiment 29. A compound of Embodiment 28 wherein R¹, R², and R³ are each independently hydrogen, halogen or C₁-C₂ haloalkyl.
Embodiment 30. A compound of Embodiment 29 wherein R¹, R², and R³ are each independently hydrogen or halogen.
Embodiment 31. A compound of Formula 1 or any one of Embodiments 1 through 30 wherein X is 0.
Embodiment 32. A compound of Formula 1 or any one of Embodiments 1 through 30 wherein X is S.
Embodiment 33. A compound of Formula 1 or any one of Embodiments 1 through 32 wherein J is C(═Z)NR⁴R⁵.
Embodiment 34. A compound of Formula 1 or any one of Embodiments 1 through 32 wherein J is CH(R⁶)N(R¹³)C(═Z)R¹⁴.
Embodiment 35. A compound of Embodiment 33 wherein Z is 0.
Embodiment 36. A compound of Embodiment 33 wherein Z is S.
Embodiment 37. A compound of Embodiment 34 wherein Z is 0.
Embodiment 38. A compound of Embodiment 34 wherein Z is S.
Embodiment 39. A compound of Formula 1 or any of the preceding Embodiments wherein R⁴ is hydrogen, C₁-C₆ alkyl, C₄-C₇ alkylcycloalkyl, C₄-C₇ cycloalkylalkyl, C₂-C₇ alkylcarbonyl or C₂-C₇ alkoxycarbonyl.
Embodiment 40. A compound of Embodiment 39 wherein R⁴ is hydrogen, C₁-C₄ alkyl, C₂-C₇ alkylcarbonyl or C₂-C₇ alkoxycarbonyl.
Embodiment 41. A compound of Embodiment 40 wherein R⁴ is hydrogen, C₂-C₇ alkylcarbonyl or C₂-C₇ alkoxycarbonyl.
Embodiment 42. A compound of Embodiment 41 wherein R⁴ is C₁-C₄ alkyl, C₂-C₇ alkylcarbonyl or C₂-C₇ alkoxycarbonyl.
Embodiment 43. A compound of Embodiment 42 wherein R⁴ is hydrogen.
Embodiment 44. A compound of Formula 1 or any of the preceding Embodiments wherein R⁵ is hydrogen, OR¹⁰, NR¹¹R¹², SO₂NR¹¹R¹², C(R¹²)═NOR¹¹, CHR¹²NHR¹¹ or Q¹; or C₁-C₆ alkyl, C₂-C₆ alkenyl, C₂-C₆ alkynyl, C₃-C₆ cycloalkyl or C₄-C₇ cycloalkylalkyl, each optionally substituted with one or more substituents independently selected from R⁷.
Embodiment 45. A compound of Embodiment 44 wherein R⁵ is hydrogen, SO₂NR¹¹R¹², C(R¹²)═NOR¹¹, CHR¹²NHR¹¹ or Q¹; or C₁-C₆ alkyl, C₂-C₆ alkenyl, C₂-C₆ alkynyl, C₃-C₆ cycloalkyl or C₄-C₇ cycloalkylalkyl, each optionally substituted with one or more substituents independently selected from R⁷.
Embodiment 46. A compound of Embodiment 45 wherein R⁵ is hydrogen or Q¹; or C₁-C₆ alkyl, C₃-C₆ cycloalkyl or C₄-C₇ cycloalkylalkyl, each optionally substituted with one or more substituents independently selected from R⁷.
Embodiment 47. A compound of Embodiment 46 wherein R⁵ is hydrogen or Q¹; or C₁-C₄ alkyl, C₃-C₅ cycloalkyl or C₄-C₆ cycloalkylalkyl, each optionally substituted with one or more substituents independently selected from R⁷.
Embodiment 48. A compound of Embodiment 47 wherein R⁵ is hydrogen.
Embodiment 49. A compound of Embodiment 47 wherein R⁵ is Q¹; or C₁-C₄ alkyl, C₃-C₅ cycloalkyl or C₄-C₆ cycloalkylalkyl, each optionally substituted with one or more substituents independently selected from R⁷.
Embodiment 50. A compound of Embodiment 49 wherein R⁵ is Q¹; or C₁-C₄ alkyl, cyclopropyl or cyclopropylmethyl, each optionally substituted with one or more substituents independently selected from R⁷.
Embodiment 51. A compound of Embodiment 50 wherein R⁵ is Q¹.
Embodiment 52. A compound of Embodiment 50 wherein R⁵ is C₁-C₄ alkyl, optionally substituted with one or more substituents independently selected from R⁷.
Embodiment 53. A compound of Embodiment 50 wherein R⁵ is cyclopropyl or cyclopropylmethyl.
Embodiment 54. A compound of Embodiment 53 wherein R⁵ is cyclopropyl.
Embodiment 55. A compound of Embodiment 53 wherein R⁵ is cyclopropylmethyl.
Embodiment 56. A compound of Formula 1 or any of the preceding Embodiments wherein R⁴ and R⁵ are taken together with the nitrogen to which they are attached to form a 3- to 6-membered ring containing ring members selected from carbon atoms and up to one additional atom independently selected from nitrogen, sulfur and oxygen, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro.
Embodiment 57. A compound of Embodiment 56 wherein R⁴ and R⁵ are taken together with the nitrogen to which they are attached to form a 3- to 6-membered ring containing ring members selected from carbon atoms and up to one additional atom independently selected from nitrogen, sulfur and oxygen, said ring optionally substituted with 1 to 2 substituents independently selected from halogen, C₁-C₂ alkyl, C₁-C₂ haloalkyl, C₁-C₂ alkoxy, C₁-C₂ haloalkoxy, cyano and nitro.
Embodiment 58. A compound of Formula 1 or any of the preceding Embodiments wherein R⁶ is hydrogen, cyano, C₁-C₃ alkyl or C₁-C₃ haloalkyl.
Embodiment 59. A compound of Embodiment 58 wherein R⁶ is hydrogen, C₁-C₃ alkyl or C₁-C₃ haloalkyl.
Embodiment 60. A compound of Embodiment 59 wherein R⁶ is hydrogen.
Embodiment 61. A compound of Formula 1 or any of the preceding Embodiments wherein each R⁷ is independently halogen, cyano, C₁-C₆ alkyl, C₁-C₆ alkoxy, C₁-C₆ alkylthio, C₁-C₆ alkylsulfinyl, C₁-C₆ alkylsulfonyl, C₁-C₆ alkylamino, C₂-C₈ dialkylamino, C₃-C₆ cycloalkylamino, C₂-C₇ alkylcarbonyl, C₂-C₇ alkoxycarbonyl, C₂-C₇ alkylaminocarbonyl, C₃-C₇ cycloalkylaminocarbonyl, C₃-C₇ alkenylaminocarbonyl, C₃-C₇ alkynylaminocarbonyl, C₃-C₉ dialkylaminocarbonyl, C₂-C₇ haloalkylcarbonyl, C₂-C₇ haloalkoxycarbonyl, C₂-C₇ haloalkylaminocarbonyl, C₃-C₉ halodialkylaminocarbonyl or —CONH₂.
Embodiment 62. A compound of Embodiment 61 wherein each R⁷ is independently halogen, cyano, C₁-C₆ alkyl, C₁-C₆ alkoxy, C₁-C₆ alkylthio, C₁-C₆ alkylsulfinyl, C₁-C₆ alkylsulfonyl, C₂-C₇ alkylcarbonyl, C₂-C₇ alkoxycarbonyl, C₂-C₇ alkylaminocarbonyl, C₃-C₇ cycloalkylaminocarbonyl, C₂-C₇ haloalkylcarbonyl, C₂-C₇ haloalkoxycarbonyl, C₂-C₇ haloalkylaminocarbonyl or —CONH₂.
Embodiment 63. A compound of Embodiment 62 wherein each R⁷ is independently C₁-C₆ alkyl, C₁-C₆ alkoxy, C₁-C₆ alkylthio, C₁-C₆ alkylsulfinyl, C₁-C₆ alkylsulfonyl, C₂-C₇ alkylcarbonyl, C₂-C₇ alkoxycarbonyl, C₂-C₇ alkylaminocarbonyl, C₃-C₇ cycloalkylaminocarbonyl or —CONH₂.
Embodiment 64. A compound of Embodiment 63 wherein each R⁷ is independently C₁-C₄ alkyl, C₁-C₄ alkoxy, C₁-C₄ alkylthio, C₁-C₄ alkylsulfinyl, C₁-C₄ alkylsulfonyl, C₂-C₅ alkylcarbonyl, C₂-C₅ alkoxycarbonyl, C₂-C₅ alkylaminocarbonyl, C₃-C₅ cycloalkylaminocarbonyl or —CONH₂.
Embodiment 65. A compound of Formula 1 or any of the preceding Embodiments wherein Q¹ is a 5- or 6-membered aromatic ring or a 4- to 6-membered saturated or unsaturated ring, each optionally containing up to three heteroatoms selected from up to 1 oxygen, up to 1 sulfur and up to 3 nitrogen, wherein up to 2 carbon atom ring members are independently selected from C(═O) and C(═S) and the sulfur atom ring member is selected from S, S(═O) and S(═O)₂, each ring optionally substituted with one or more substituents independently selected from R⁸.
Embodiment 66. A compound of Embodiment 65 wherein Q¹ is a 5- or 6-membered aromatic ring, optionally containing up to three heteroatoms selected from up to 1 oxygen, up to 1 sulfur and up to 3 nitrogen, wherein up to 1 carbon atom ring member is C(═O), each ring optionally substituted with one or more substituents independently selected from R⁸.
Embodiment 67. A compound of Embodiment 66 wherein Q¹ is a 4- to 6-membered saturated or unsaturated ring, optionally containing up to two heteroatoms selected from up to 1 oxygen, up to 1 sulfur and up to 2 nitrogen, wherein up to 1 carbon atom ring member is C(═O) and the sulfur atom ring member is selected from S, S(═O) and S(═O)₂, each ring optionally substituted with one or more substituents independently selected from R⁸.
Embodiment 68. A compound of Embodiment 67 wherein Q¹ is a 4- to 6-membered saturated ring, optionally containing up to two heteroatoms selected from up to 1 oxygen, up to 1 sulfur and up to 2 nitrogen, wherein the sulfur atom ring member is selected from S, S(═O) and S(═O)₂.
Embodiment 69. A compound of Formula 1 or any of the preceding Embodiments wherein each Q² is independently a phenyl ring, optionally substituted with one or more substituents independently selected from R⁹.
Embodiment 70. A compound of Formula 1 or any of the preceding Embodiments wherein each Q² is a 5- or 6-membered aromatic heterocyclic ring, each ring optionally substituted with one or more substituents independently selected from R⁹.
Embodiment 71. A compound of Formula 1 or any of the preceding Embodiments wherein each Q² is independently a 3- to 6-membered non-aromatic heterocyclic ring, each ring optionally substituted with one or more substituents independently selected from R⁹.
Embodiment 72. A compound of Formula 1 or any of the preceding Embodiments wherein each R⁸ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, C₂-C₇ alkylaminocarbonyl, C₃-C₉ dialkylaminocarbonyl, cyano or nitro.
Embodiment 73. A compound of Embodiment 72 wherein each R⁸ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy or cyano.
Embodiment 74. A compound of Embodiment 73 wherein each R⁸ is independently halogen, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy or cyano.
Embodiment 75. A compound of Embodiment 74 wherein each R⁸ is independently halogen, C₁-C₄ alkyl, C₁-C₄ haloalkyl, C₁-C₄ haloalkoxy or cyano.
Embodiment 76. A compound of Formula 1 or any of the preceding Embodiments wherein each R⁹ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy or cyano.
Embodiment 77. A compound of Embodiment 76 wherein each R⁹ is independently halogen, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy or cyano.
Embodiment 78. A compound of Embodiment 77 wherein each R⁹ is independently halogen, C₁-C₄ alkyl, C₁-C₄ haloalkyl, C₁-C₄ haloalkoxy or cyano.
Embodiment 79. A compound of Formula 1 or any of the preceding Embodiments wherein R¹⁰ is hydrogen; or C₁-C₆ alkyl, C₃-C₆ cycloalkyl, C₄-C₇ alkylcycloalkyl or C₄-C₇ cycloalkylalkyl, each optionally substituted with one or more halogen.
Embodiment 80. A compound of Embodiment 79 wherein R¹⁰ is hydrogen; or C₁-C₆ alkyl, C₃-C₆ cycloalkyl, C₄-C₇ alkylcycloalkyl or C₄-C₇ cycloalkylalkyl, each optionally substituted with one to three halogen.
Embodiment 81. A compound of Embodiment 80 wherein R¹⁰ is C₃-C₆ cycloalkyl, C₄-C₇ alkylcycloalkyl or C₄-C₇ cycloalkylalkyl, each optionally substituted with one to three halogen.
Embodiment 82. A compound of Embodiment 80 wherein R¹⁰ is hydrogen; or C₁-C₆ alkyl optionally substituted with one to three halogen.
Embodiment 83. A compound of Formula 1 or any of the preceding Embodiments wherein R¹¹ is hydrogen, C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl.
Embodiment 84. A compound of Formula 1 or any one of Embodiments 1 through 82 wherein R¹¹ is hydrogen, C₁-C₆ alkyl, C₂-C₇ alkylcarbonyl, C₂-C₇ haloalkylcarbonyl or C₂-C₇ alkoxycarbonyl.
Embodiment 85. A compound of Embodiment 84 wherein R¹¹ is hydrogen, C₁-C₄ alkyl, C₂-C₅ alkylcarbonyl, C₂-C₅ haloalkylcarbonyl or C₂-C₅ alkoxycarbonyl.
Embodiment 86. A compound of Embodiment 85 wherein R¹¹ is hydrogen.
Embodiment 87. A compound of Formula 1 or any of the preceding Embodiments wherein R¹² is hydrogen; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷.
Embodiment 88. A compound of Embodiment 87 wherein R¹² is hydrogen; or C₁-C₆ alkyl, C₃-C₆ cycloalkyl, each optionally substituted with one to three substituents independently selected from R⁷.
Embodiment 89. A compound of Embodiment 88 wherein R¹² is hydrogen; or C₁-C₄ alkyl, each optionally substituted with one to three substituents independently selected from R⁷.
Embodiment 90. A compound of Embodiment 89 wherein R¹² is C₁-C₄ alkyl.
Embodiment 91. A compound of Formula 1 or any of the preceding Embodiments wherein R¹¹ and R¹² are taken together with the nitrogen to which they are attached to form a 3- to 6-membered ring containing ring members selected from carbon atoms and up to one additional atom independently selected from nitrogen, sulfur and oxygen, said ring optionally substituted with 1 to 2 substituents independently selected from the halogen, C₁-C₂ alkyl, C₁-C₂ haloalkyl, C₁-C₂ alkoxy, C₁-C₂ haloalkoxy, cyano and nitro.
Embodiment 92. A compound of Embodiment 91 wherein R¹¹ and R¹² are taken together with the nitrogen to which they are attached to form a 3- to 6-membered ring containing ring members selected from carbon atoms and up to one additional atom independently selected from nitrogen, sulfur and oxygen, said ring optionally substituted with 1 to 2 substituents independently selected from halogen, C₁-C₂ alkyl and C₁-C₂ haloalkyl.
Embodiment 93. A compound of Formula 1 or any of the preceding Embodiments wherein Q³ is a phenyl ring, optionally substituted with one to three substituents independently selected from R⁹.
Embodiment 94. A compound of Formula 1 or any of the preceding Embodiments wherein Q³ is a 5- or 6-membered heterocyclic ring, optionally substituted with one to three substituents independently selected from R⁹.
Embodiment 95. A compound of Formula 1 or any of the preceding Embodiments wherein R¹³ is hydrogen, C₁-C₆ alkyl, C₂-C₇ alkylcarbonyl, C₂-C₄ haloalkylcarbonyl or C₂-C₇ alkoxycarbonyl.
Embodiment 96. A compound of Embodiment 95 wherein R¹³ is hydrogen, C₁-C₄ alkyl, C₂-C₇ alkylcarbonyl or C₂-C₇ alkoxycarbonyl.
Embodiment 97. A compound of Embodiment 96 wherein R¹³ is hydrogen, C₂-C₇ alkylcarbonyl or C₂-C₇ alkoxycarbonyl.
Embodiment 98. A compound of Embodiment 97 wherein R¹³ is hydrogen.
Embodiment 99. A compound of Formula 1 or any of the preceding Embodiments wherein R¹⁴ is C₁-C₆ alkyl optionally substituted with halogen.
Embodiment 100. A compound of Embodiment 99 wherein R¹⁴ is C₁-C₄ alkyl optionally substituted with halogen.
Embodiment 101. A compound of Embodiment 100 wherein R¹⁴ is methyl or ethyl.
Embodiment 102. A compound of Formula 1 or any of the preceding Embodiments wherein R¹⁴ is C₁-C₆ alkyl optionally substituted with OR¹⁹.
Embodiment 103. A compound of Formula 1 or any of the preceding Embodiments wherein R¹⁴ is C₁-C₆ alkyl optionally substituted with S(═O)ₙR²⁰.
Embodiment 104. A compound of Formula 1 or any of the preceding Embodiments wherein R¹⁴ is C₁-C₆ alkyl optionally substituted with NR²¹C(═O)R²².
Embodiment 105. A compound of Formula 1 or any one of Embodiments 1 through 98 wherein R¹⁴ is C₃-C₆ cycloalkyl or C₄-C₇ cycloalkylalkyl, each optionally substituted with up to 2 substituents selected from halogen, cyano, C₁-C₂ alkyl and C₁-C₂ haloalkyl.
Embodiment 106. A compound of Formula 1 or any one of Embodiments 1 through 98 wherein R¹⁴ is C₃-C₆ cycloalkyl, optionally substituted with up to one cyclopropyl and up to 2 substituents selected from halogen, cyano, C₁-C₂ alkyl and C₁-C₂ haloalkyl.
Embodiment 107. A compound of Formula 1 or any one of Embodiments 1 through 98 wherein R¹⁴ is C₄-C₇ cycloalkylalkyl, optionally substituted with up to one cyclopropyl and up to 2 substituents selected from halogen, cyano, C₁-C₂ alkyl and C₁-C₂ haloalkyl.
Embodiment 108. A compound of Formula 1 or any one of Embodiments 1 through 98 wherein R¹⁴ is OR¹⁶.
Embodiment 109. A compound of Formula 1 or any one of Embodiments 1 through 98 wherein R¹⁴ is NR^{17a}R^{17b}.
Embodiment 110. A compound of Formula 1 or any of the preceding Embodiments wherein Q⁴ is a 3- to 6-membered saturated heterocyclic ring containing ring members selected from carbon atoms and one heteroatom independently selected from one oxygen and one sulfur, optionally substituted with up to 2 substituents independently selected from R¹⁸.
Embodiment 111. A compound of Formula 1 or any of the preceding Embodiments wherein R¹⁶ is C₁-C₂ alkyl or C₁-C₂ haloalkyl.
Embodiment 112. A compound of Formula 1 or any of the preceding Embodiments wherein R^{17a} is hydrogen, C₁-C₂ alkyl, C₁-C₂ haloalkyl or C₃-C₅ cycloalkyl.
Embodiment 113. A compound of Embodiment 112 wherein R^{17a} is C₁-C₂ alkyl or C₁-C₂ haloalkyl.
Embodiment 114. A compound of Embodiment 112 wherein R^{17a} is hydrogen.
Embodiment 115. A compound of Formula 1 or any of the preceding Embodiments wherein R^{17b} is hydrogen, C₁-C₂ alkyl, C₁-C₂ haloalkyl or C₃-C₅ cycloalkyl.
Embodiment 116. A compound of Embodiment 115 wherein R^{17b} is hydrogen, C₁-C₂ alkyl or C₁-C₂ haloalkyl.
Embodiment 117. A compound of Embodiment 116 wherein R^{17b} is hydrogen or C₁-C₄ alkyl.
Embodiment 118. A compound of Embodiment 117 wherein R^{17b} is hydrogen.
Embodiment 119. A compound of Formula 1 or any of the preceding Embodiments wherein R^{17a} and R^{17b} are taken together with the nitrogen to which they are attached to form a 3- to 6-membered ring containing ring members selected from carbon atoms and up to one additional atom independently selected from nitrogen, sulfur and oxygen, said ring optionally substituted with 1 to 2 substituents independently selected from halogen, C₁-C₂ alkyl, C₁-C₂ haloalkyl, C₁-C₂ alkoxy, C₁-C₂ haloalkoxy, cyano and nitro.
Embodiment 120. A compound of Embodiment 119 wherein R^{17a} and R^{17b} are taken together with the nitrogen to which they are attached to form a 3- to 6-membered ring containing ring members selected from carbon atoms and up to one additional atom independently selected from nitrogen, sulfur and oxygen, said ring optionally substituted with 1 to 2 substituents independently selected from halogen, C₁-C₂ alkyl and C₁-C₂ haloalkyl.
Embodiment 121. A compound of Formula 1 or any of the preceding Embodiments wherein each R¹⁸ is independently halogen, C₁-C₂ alkyl or C₁-C₂ haloalkyl.
Embodiment 122. A compound of Formula 1 or any of the preceding Embodiments wherein R¹⁹ is hydrogen, C₁-C₂ alkyl or C₁-C₂ haloalkyl.
Embodiment 123. A compound of Formula 1 or any of the preceding Embodiments wherein R²⁰ is C₁-C₂ alkyl or C₁-C₂ haloalkyl.
Embodiment 123. A compound of Formula 1 or any of the preceding Embodiments wherein R²¹ is hydrogen or C₁-C₂ alkyl.
Embodiment 124. A compound of Formula 1 or any of the preceding Embodiments wherein R²² is C₁-C₂ alkyl, C₁-C₂ haloalkyl or C₃-C₆ cycloalkyl.
Embodiment 125. A compound of Formula 1 or any of the preceding Embodiments wherein n is 0.
Embodiment 126. A compound of Formula 1 or any of the preceding Embodiments wherein n is 1 or 2.
Embodiment 127. A compound of Embodiment 126 wherein n is 1.
Embodiment 128. A compound of Embodiment 126 wherein n is 2.
Embodiment 129. A compound of Formula 1 or any of the preceding Embodiments wherein m is 0.
Embodiment 130. A compound of Formula 1 or any of the preceding Embodiments wherein m is 1.
Embodiment S1. A compound of any one of Embodiments 1-130 wherein the compound of Formula 1 is a compound of Formula 1′.
Embodiment S2. A compound of any one of Embodiments 1-130 wherein the compound of Formula 1 is a compound of Formula 1″.
Embodiment S3. A composition consisting of a compound of Formula 1′ and a compound of Formula 1″ wherein the ratio of the compound of Formula 1′ to the compound of Formula 1″ is greater than 60:40.
Embodiment S3a. A composition of Embodiment S3 wherein the ratio of the compound of Formula 1′ to the compound of Formula 1″ is greater than 80:20.
Embodiment S3b. A composition of Embodiment S3 wherein the ratio of the compound of Formula 1′ to the compound of Formula 1″ is greater than 90:10.
Embodiment S3c. A composition of Embodiment S3 wherein the ratio of the compound of Formula 1′ to the compound of Formula 1″ is greater than 99:1.
Embodiment S4. A composition consisting of a compound of Formula 1″ and a compound of Formula 1′ wherein the ratio of the compound of Formula 1″ to the compound of Formula 1′ is greater than 60:40.

Embodiment X. A method for controlling an invertebrate pest comprising contacting the invertebrate pest or its environment with a biologically effective amount of a compound of Formula 1.
Embodiment X1. The method of Claim X wherein the environment is soil or plant foliage.
Embodiment Y1. A composition comprising a compound of Formula 1 or any one of the preceding embodiments and at least one additional component selected from the group consisting of surfactants, solid diluents and liquid diluents, said composition optionally further comprising at least one additional biologically active compound or agent.
Embodiment Y2. The composition of embodiment Y1 wherein the at least one additional biologically active compound or agent is selected from the group consisting of abamectin, acephate, acequinocyl, acetamiprid, acrinathrin, afidopyropen, amidoflumet, amitraz, avermectin, azadirachtin, azinphos-methyl, benfuracarb, bensultap, bifenthrin, bifenazate, bistrifluron, borate, bromantraniliprole, buprofezin, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cyclaniliprole, cycloprothrin, cycloxaprid, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyromazine, deltamethrin, diafenthiuron, diazinon, dichlorantraniliprole, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin, flonicamid, flubendiamide, flucythrinate, flufenerim, flufenoxuron, flufenoxystrobin, fluensulfone, flupiprole, fluopyram, flupyradifurone, fluvalinate, tau-fluvalinate, fonophos, formetanate, fosthiazate, halofenozide, heptafluthrin, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicotine, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide, N-[1-(difluoromethyl)cyclopropyl]-2-(3-pyridinyl)-2H-indazole-4-carboxamide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, tetrachlorantraniliprole, tetrachlorvinphos, tetramethrin, tetramethylfluthrin, thiacloprid, thiamethoxam, thiodicarb, thiosultap-sodium, tioxazafen, tolfenpyrad, tralomethrin, triazamate, trichlorfon, triflumezopyrim, triflumuron, Bacillus thuringiensis delta-endotoxins, all strains of Bacillus thuringiensis, all strains of nuclear polyhedrosis viruses entomopathogenic bacteria, entomopathogenic viruses and entomopathogenic fungi.
Embodiment Y3. The composition of embodiment Y2 wherein the at least one additional biologically active compound or agent is selected from the group consisting of abamectin, acetamiprid, acrinathrin, afidopyropen, amitraz, avermectin, azadirachtin, benfuracarb, bensultap, bifenthrin, buprofezin, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, fluensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nuclear polyhedrosis viruses.
Embodiment Y4. The composition of any one of embodiments Y1-Y3 further comprising a liquid fertilizer.
Embodiment Y5. The composition of Embodiment Y4 wherein the liquid fertilizer is aqueous-based.
Embodiment Y6. A soil drench formulation comprising the composition of any one of embodiments Y1-Y3.
Embodiment Y7. A spray composition comprising the composition of any one of embodiments Y1-Y3 and a propellant.
Embodiment Y8. A bait composition, comprising the composition of any one of embodiments Y1-Y3, one or more food materials, optionally an attractant, and optionally a humectant.
Embodiment Y9. A trap device for controlling an invertebrate pest, comprising: the bait composition of Embodiment Y8 and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.
Embodiment Y10. A composition comprising the composition of any of Embodiments Y1-Y3 wherein the composition is a solid composition selected from dusts, powders, granules, pellets, prills, pastilles, tablets, and filled films.
Embodiment Y11. The composition of Embodiment Y10 wherein the composition is water-dispersible or water-soluble.
Embodiment Y12. A liquid or dry formulation comprising the composition of any one of Embodiments Y1-Y3 for use in a drip irrigation system, furrow during planting, handheld sprayer, backpack sprayer, boom sprayer, ground sprayer, aerial application, unmanned aerial vehicle, or a seed treatment.
Embodiment Y13. The liquid or dry formulation of Embodiment Y12 wherein said formulation is sprayed at an ultra-low volume.

Embodiments of this disclosure, including Embodiments 1-Y13 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this disclosure, including Embodiments 1-Y13 above as well as any other embodiments described herein, and any combination thereof, pertain to the compositions and methods of the present disclosure.

Combinations of Embodiments 1-Y13 are illustrated by:

Embodiment A. A compound of Formula 1 wherein
A is O; and
R¹, R², and R³ are each independently hydrogen, halogen, cyano, C₁-C₄ alkyl, C₁-C₄ haloalkyl, C₁-C₄ alkoxy or C₁-C₄ haloalkoxy.

Embodiment B. A compound of Embodiment A wherein
T is T-1, T-2, T-3, T-4, T-5, T-6 or T-9;
R¹, R², and R³ are each independently hydrogen, halogen, C₁-C₂ alkyl, C₁-C₂ haloalkyl, C₁-C₂ alkoxy or C₁-C₂ haloalkoxy; and
Z is O.

Embodiment C. A compound of Embodiment B wherein
is T-1, T-2, T-3, T-6 or T-9;
R¹, R², and R³ are each independently hydrogen, halogen or C₁-C₂ haloalkyl;
R⁴ is hydrogen, C₂-C₇ alkylcarbonyl or C₂-C₇ alkoxycarbonyl; and
R⁶ is hydrogen, C₁-C₃ alkyl or C₁-C₃ haloalkyl.

Embodiment D. A compound of Embodiment C wherein
R⁴ is hydrogen;
R⁵ is hydrogen or Q¹; or C₁-C₆ alkyl, C₃-C₆ cycloalkyl or C₄-C₇ cycloalkylalkyl, each optionally substituted with one or more substituents independently selected from R⁷;
R⁶ is hydrogen;
each R⁷ is independently C₁-C₄ alkyl, C₁-C₄ alkoxy, C₁-C₄ alkylthio, C₁-C₄ alkylsulfinyl, C₁-C₄ alkylsulfonyl, C₂-C₅ alkylcarbonyl, C₂-C₅ alkoxycarbonyl, C₂-C₅ alkylaminocarbonyl, C₃-C₅ cycloalkylaminocarbonyl or —CONH₂;
Q¹ is a 4- to 6-membered saturated or unsaturated ring, optionally containing up to two heteroatoms selected from up to 1 oxygen, up to 1 sulfur and up to 2 nitrogen, wherein up to 1 carbon atom ring member is C(═O) and the sulfur atom ring member is selected from S, S(═O) and S(═O)₂, each ring optionally substituted with one or more substituents independently selected from R⁸;
R¹³ is hydrogen, C₁-C₄ alkyl, C₂-C₇ alkylcarbonyl or C₂-C₇ alkoxycarbonyl; and
R¹⁴ is C₁-C₆ alkyl optionally substituted with halogen.

Embodiment E. A compound of Embodiment D wherein
R⁵ is Q¹; or C₁-C₄ alkyl, cyclopropyl or cyclopropylmethyl, each optionally substituted with one or more substituents independently selected from R⁷;
Q¹ is a 4- to 6-membered saturated ring, optionally containing up to two heteroatoms selected from up to 1 oxygen, up to 1 sulfur and up to 2 nitrogen, wherein the sulfur atom ring member is selected from S, S(═O) and S(═O)₂;
R¹³ is hydrogen; and
R¹⁴ is methyl or ethyl.

Embodiment F. A compound of Embodiment E wherein
T is T-1, T-2 or T-6.

Embodiment G. A compound of Formula 1 or any one of Embodiments A-F wherein the compound of Formula 1 is Formula 1′.

Embodiment H. A compound of Formula 1 or any one of Embodiments A-F wherein the compound of Formula 1 is Formula 1″.

Specific embodiments include compounds of Formula 1 selected from the group consisting of:
N-(cyclopropylmethyl)-4-[5-(3,5-dichloro-4-fluorophenyl)-4,5-dihydro-5-(trifluoromethyl)-3-isoxazolyl]furo[2,3-c]pyridine-7-carboxamide;
N-cyclopropyl-4-[5-(3,5-dichloro-4-fluorophenyl)-4,5-dihydro-5-(trifluoro-methyl)-3-isoxazolyl]furo[2,3-c]pyridine-7-carboxamide;
N-cyclopropyl-7-[5-(3,5-dichlorophenyl)-4,5-dihydro-5-(trifluoromethyl)-3-isoxazolyl]thieno[2,3-c]pyridine-4-carboxamide;
7-[5-(3,5-dichlorophenyl)-4,5-dihydro-5-(trifluoromethyl)-3-isoxazolyl]-N-[(1R)-1-methyl-2-(methylamino)-2-oxoethyl]thieno[2,3-c]pyridine-4-carboxamide;
7-[5-(3,5-dichlorophenyl)-4,5-dihydro-5-(trifluoromethyl)-3-isoxazolyl]-N-(1,1-dioxido-3-thietanyl)thieno[2,3-c]pyridine-4-carboxamide;
N-[[4-[4,5-dihydro-5-(trifluoromethyl)-5-[3-(trifluoromethyl)phenyl]-3-isoxazolyl]furo[2,3-c]pyridin-7-yl]methyl]acetamid;
N-[[4-[4,5-dihydro-5-(trifluoromethyl)-5-[3-(trifluoromethyl)phenyl]-3-isoxazolyl]furo[2,3-c]pyridin-7-yl]methyl]propanamid;
4-[5-(3,5-dichlorophenyl)-4,5-dihydro-5-(trifluoromethyl)-3-isoxazolyl]-N-[(1R)-1-methyl-2-(methylamino)-2-oxoethyl]thieno[3,2-c]pyridine-7-carboxamide;
N-cyclopropyl-4-[5-(3,5-dichlorophenyl)-4,5-dihydro-5-(trifluoromethyl)-3-isoxazolyl]thieno[3,2-c]pyridine-7-carboxamide;
4-[5-(3,5-dichlorophenyl)-4,5-dihydro-5-(trifluoromethyl)-3-isoxazolyl]-N-(1,1-dioxido-3-thietanyl)thieno[3,2-c]pyridine-7-carboxamide;
N-[[4-[4,5-dihydro-5-(trifluoromethyl)-5-[3-(trifluoromethyl)phenyl]-3-isoxazolyl]-2,3-dihydrofuro[2,3-c]pyridin-7-yl]methyl]-propanamide;
4-[5-(3,5-dichloro-4-fluorophenyl)-4,5-dihydro-5-(trifluoromethyl)-3-isoxazolyl]-6,7-dihydro-N-[(1R)-1-methyl-2-(methylamino)-2-oxoethyl]-5H-cyclopenta[c]pyridine-1-carboxamide.

Of note is that compounds of this disclosure are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the disclosure. Compounds of this disclosure because of their favorable translocation properties or systemicity in plants also protect foliar or other plant parts which are not directly contacted with a compound of Formula 1 or a composition comprising the compound.

Also noteworthy as embodiments of the present disclosure are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present disclosure are compositions for controlling an invertebrate pest comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the disclosure further include methods for controlling an invertebrate pest comprising contacting the invertebrate pest or its environment with a biologically effective amount of a compound of any of the preceding Embodiments (e.g., as a composition described herein).

Embodiments of the disclosure also include a composition comprising a compound of any of the preceding Embodiments, in the form of a soil drench liquid formulation. Embodiments of the disclosure further include methods for controlling an invertebrate pest comprising contacting the soil with a liquid composition as a soil drench comprising a biologically effective amount of a compound of any of the preceding Embodiments.

Embodiments of the disclosure also include a spray composition for controlling an invertebrate pest comprising a biologically effective amount of a compound of any of the preceding Embodiments and a propellant. Embodiments of the disclosure further include a bait composition for controlling an invertebrate pest comprising a biologically effective amount of a compound of any of the preceding Embodiments, one or more food materials, optionally an attractant, and optionally a humectant. Embodiments of the disclosure also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the disclosure also include methods for protecting a seed from an invertebrate pest comprising contacting the seed with a biologically effective amount of a compound of any of the preceding Embodiments.

Embodiments of the disclosure also include methods for protecting an animal from an invertebrate parasitic pest comprising administering to the animal a parasiticidally effective amount of a compound of any of the preceding Embodiments.

Embodiments of the disclosure also include methods for controlling an invertebrate pest comprising contacting the invertebrate pest or its environment with a biologically effective amount of a compound of Formula 1, an N-oxide or a salt thereof, (e.g., as a composition described herein), provided that the methods are not methods of medical treatment of a human or animal body by therapy.

This disclosure also relates to such methods wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

Embodiments of this disclosure also include use of an unmanned aerial vehicle (UAV) for the dispersion of the compositions disclosed herein over a planted area. In some embodiments the planted area is a crop-containing area. In some embodiments, the crop is selected from a monocot or dicot. In some embodiments, the crop is selected form rice, corn, barley, soybean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally is 5.0 liters/ha-100 liters/ha (approximately 0.5-10 gpa). This includes the range of ultra-low spray volume (ULV) to low spray volume (LV). Although not common there may be situations where even lower spray volumes could be used as low as 1.0 liter/ha (0.1 gpa).

The compounds of Formula 1 can be prepared by one or more of the following methods and variations as described in Schemes 1-32. The definitions of substituents in the compounds of Formulae 1-49 below are as defined above in the Summary unless otherwise noted. Compounds of Formulae 1a-1k are subsets of the compounds of Formula 1; Formula 5a is a subset of Formula 5; Formula 6a is a subset of Formula 6; Formula 15a is a subset of Formula 15; Formula 19a is a subset of Formula 19; Formula 27a and 27b are subsets of Formula 27; Formula 30a and 30b are subsets of Formula 30; Formula 32a is a subset of Formula 32; Formula 36a is a subset of Formula 36; Formula 38a is a subset of Formula 38; Formula 40a is a subset of Formula 40; Formula 42a is a subset of Formula 42; Formula 44a and 44b are subsets of Formula 44; and Formula 46a is a subset of Formula 46. Substituents for each subset formula are as defined for its parent formula unless otherwise noted. Ambient or room temperature is defined as about 20-25° C.

As shown in Scheme 1, compounds of Formula 1a (i.e. Formula 1 wherein T is T-2, J is C(═Z)NR⁴R⁵ and Z is O) can be prepared by reacting a carboxylic acid of Formula 2 with amines of Formula 3 in the presence of a coupling reagent. Useful coupling reagents include, for example, dicyclohexyl carbodiimide, 1-(3-dimethylaminopropyl)-3-ethylcarbodiimide and carbonyl diimidazole. Further coupling reagents useful in this method include propylphosphonic anhydride (T3P), 0-(7-azabenzotriazol-1-yl)-N,N,N′,N′-tetramethyluronium hexafluorophosphate (HATU) and N-[(1H-benzotriazol-1-yl-oxy)(dimethylamino)methylene]-N-methyl-methanaminium hexafluorophosphate (HBTU). These coupling reagents are generally used in the presence of a base such as triethylamine, pyridine, 4-(dimethylamino)pyridine or N,N-diisopropylethylamine. Typical reaction conditions include an anhydrous aprotic solvent such as dichloromethane, tetrahydrofuran or N,N-dimethylformamide, and a reaction temperature between room temperature and 70° C. For reaction conditions useful in the method of Scheme 1, as well as other well-established coupling conditions see, for example, Journal of Organic Chemistry 2008, 73(7), 2731-2737; Tetrahedron Letters 2009, 50(45), 6200-6202; and Organic letters 2011, 13(12), 2988-91. Also, present Example 1 Step I and present Example 4 Step H illustrate the method of Scheme 1.

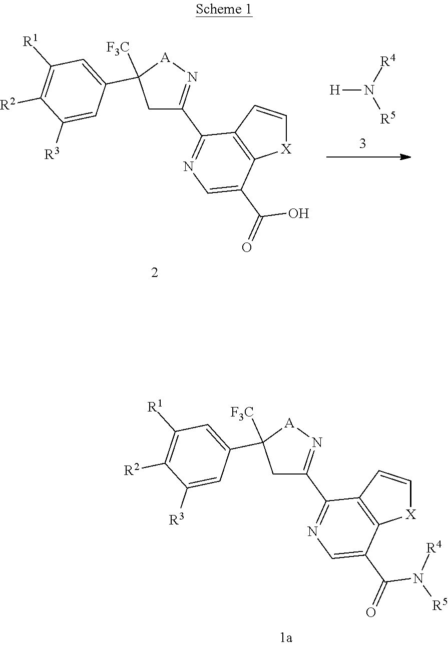

Alternatively, as shown in Scheme 2, compounds of Formula 1a can be prepared by reacting an acid chloride of Formula 4 with amines of Formula 3 in the presence of an acid scavenger. Typical acid scavengers include amine bases such as triethylamine, N,N-diisopropylethylamine and pyridine. Other scavengers include hydroxides such as sodium hydroxide and potassium hydroxide, or carbonates such as sodium carbonate and potassium carbonate. In certain instances it is useful to use polymer-supported acid scavengers such as polymer-bound N,N-diisopropylethylamine and polymer-bound 4-(dimethylamino)pyridine.

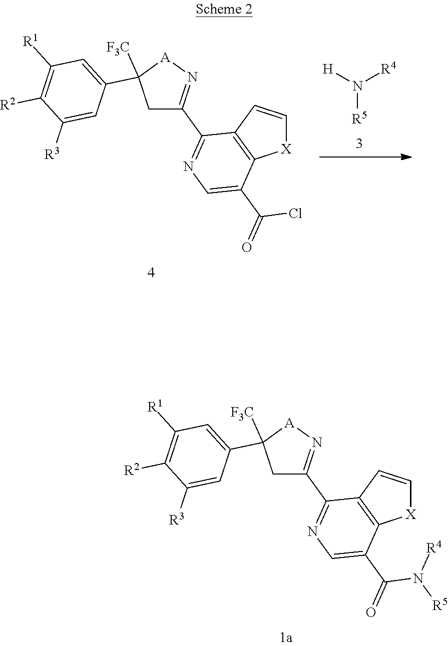

Acid chlorides of Formula 4 are easily prepared from carboxylic acids of Formula 2 by numerous well-known methods. For example, reacting the carboxylic acid with a chlorinating reagent such as thionyl chloride, oxalyl chloride or phosphorus oxychloride in a solvent such as dichloromethane or toluene and optionally in the presence of a catalytic amount of N,N-dimethylformamide can provide the corresponding acid chloride of Formula 4.

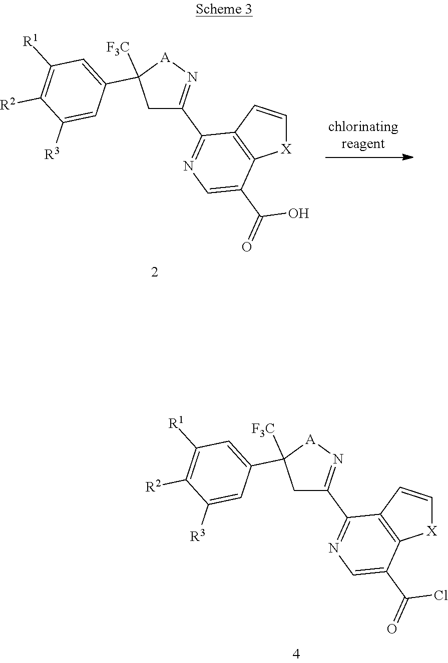

Compounds of Formula 1a can also be prepared by aminocarbonylation of aryl halides of Formula 5 wherein Y¹ is Cl, Br or I, with amines of Formula 3 as shown in Scheme 4.

This reaction is typically carried out with an aryl halide of Formula 5 wherein Y¹ is Br or I in the presence of a palladium catalyst under CO atmosphere. The palladium catalysts used for the present method typically comprises palladium in a formal oxidation state of either 0 (i.e. Pd(0)) or 2 (i.e. Pd(II)). A wide variety of such palladium-containing compounds and complexes are useful as catalysts for the present method. Examples of palladium-containing compounds and complexes useful as catalysts in the method of Scheme 4 include PdCl₂(PPh₃)₂ (i.e. bis(triphenylphosphine)palladium(II) dichloride), Pd₂(dba)₃ (i.e. tris(dibenzylideneacetone)dipalladium(0)), Pd(PPh₃)₄ (i.e. tetrakis(triphenyl-phosphine)palladium(0)), Pd(C₅H₇O₂)₂(i.e. palladium(II) acetyl-acetonate), and dichloro-[1,1′-bis(diphenylphosphino)ferrocene]palladium(II). The method of Scheme 4 is generally conducted in a liquid phase, and therefore to be most effective the palladium catalyst preferably has good solubility in the liquid phase. Useful solvents include, for example, ethers such as 1,2-dimethoxyethane, amides such as N,N-dimethylacetamide, and non-halogenated aromatic hydrocarbons such as toluene.

The method of Scheme 4 can be conducted over a wide range of temperatures, ranging from about 25 to about 150° C. Of note are temperatures from about 60 and about 110° C., which typically provide faster reaction rates and higher product yields. The general methods and procedures for aminocarbonylation with an aryl bromide and an amine are well known in the literature; see, for example, Synthesis 1989, 715; and Palladium in Heterocyclic Chemistry: A Guide for the Synthetic Chemist, 2000.

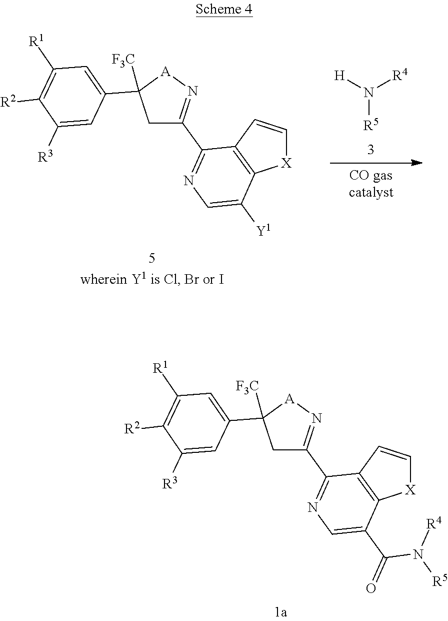

As shown in Scheme 5, carboxylic acids of Formula 2 can be prepared according to well-known methods of basic or acidic hydrolysis of the corresponding compounds of Formula 6, preferably using a slight excess of a hydroxide base (e.g. lithium hydroxide) in a water-miscible co-solvent such as methanol, ethanol or tetrahydrofuran at a temperature between about 0 and 45° C. The product can be isolated by acidification and then filtering or extracting, optionally after removal of the organic solvent by evaporation. Present Example 1 Step H and present Example 4 Step G illustrate the method of Scheme 5.

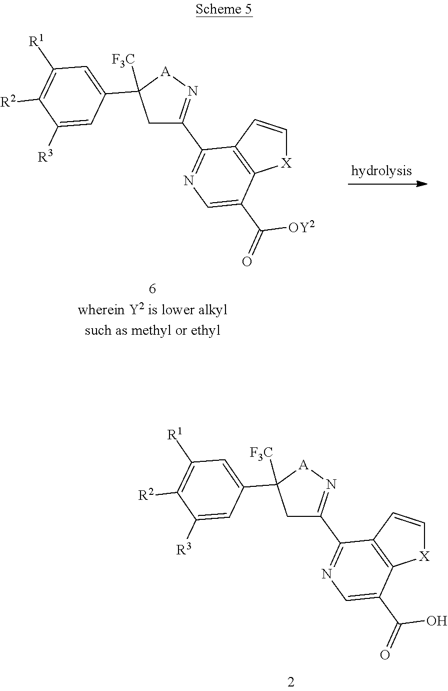

Alcohols of Formula 8 can be substituted for the amines of Formula 3 in the reaction of Scheme 4, to yield the esters of Formula 6 as shown in Scheme 6.

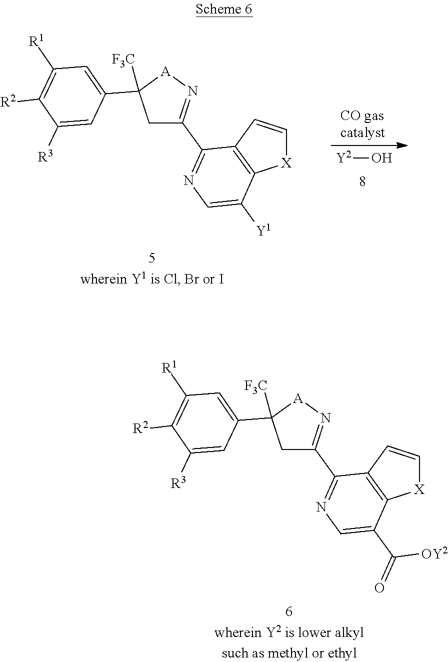

Compounds of Formula 5a (i.e. Formula 5 wherein A is O) can be prepared by a two-step procedure from aldehydes of Formula 9 as outlined in Scheme 7. In the first step, an aldehyde of Formula 9 is reacted with hydroxylamine to provide an oxime of Formula 10. General procedures for this reaction are documented in the chemical literature; see for example, Bioorg. Med. Chem. 2004, 12, 3965.

In the second step, the oxime of Formula 10 is reacted with a styrene of Formula 11 to produce compounds of Formula 5a. This reaction typically involves chlorination of the oxime of Formula 10 and subsequent dehydrochlorination to yield an in situ generated nitrile oxide, which then undergoes 1,3-dipolar cycloaddition with the styrene of Formula 11 to afford compounds of Formula 5a. In a typical procedure, a chlorinating reagent such as sodium hypochlorite, N-chlorosuccinimide or chloramine-T is combined with the oxime in the presence of the styrene. To facilitate the dehydrochlorination step it may be necessary to carry out the reaction under basic conditions. Typical bases include pyridine and triethylamine. The reaction can be run in a wide variety of solvents including tetrahydrofuran, diethyl ether, methylene chloride, dioxane, and toluene with temperatures ranging from room temperature to the reflux temperature of the solvent. General procedures for cycloaddition of nitrile oxides with olefins are well documented in the chemical literature, see for example, Synthesis, 1982, 6, 508-509; Tetrahedron, 2000, 56, 1057-1064; and EP 1,538,138-A1, as well as references cited within.

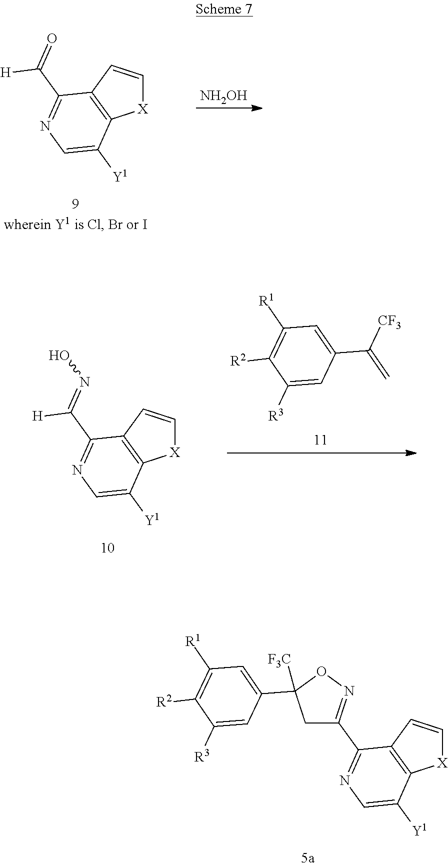

One skilled in the art will recognize that esters of Formula 6a (i.e. Formula 6 wherein A is O) can be prepared analogous to the 1,3-dipolar cycloaddition method described in Scheme 7 above where Y¹ is replaced by C(═O)OY².

Esters of Formula 6a (i.e. Formula 6 wherein A is O) can also be prepared by a two-step procedure from ketones of Formula 12 as outlined in Scheme 8. In the first step, a ketone of Formula 12 is condensed with a trifluoromethyl ketone of Formula 13 to provide a compound of Formula 14. This reaction is typically conducted in the presence of a base such as calcium hydroxide, potassium carbonate or cesium carbonate in a solvent or mixture solvents such as toluene, N,N-dimethylformamide, 2-methoxy-2-methylpropane (MTBE), (trifluoromethyl)benzene, 1,2-dichloroethane or acetonitrile. Present Example 1 Step F and present Example 4 Steps E illustrate the condensation step of the method of Scheme 8.

In the second step, the condensation product of Formula 14 is treated with hydroxylamine or a hydroxylamine salt to produce isoxazoline compounds of Formula 6a. This reaction is typically run under basic conditions. Typical bases include sodium hydroxide, lithium hydroxide or 1,8-diazabicyclo[5.4.0]undec-7-ene (DBU). The reaction can be run in a solvent or mixture solvents such as toluene, N,N-dimethylformamide, 2-methoxy-2-methylpropane (MTBE), (trifluormethyl)benzene, 1,2-dichloroethane or acetonitrile. General procedures for method of Scheme 8 are well documented in the chemical literature, see for example, WO 2009/126668. Present Example 1 Step G and present Example 4 Step F illustrate the cyclization step of the method of Scheme 8.

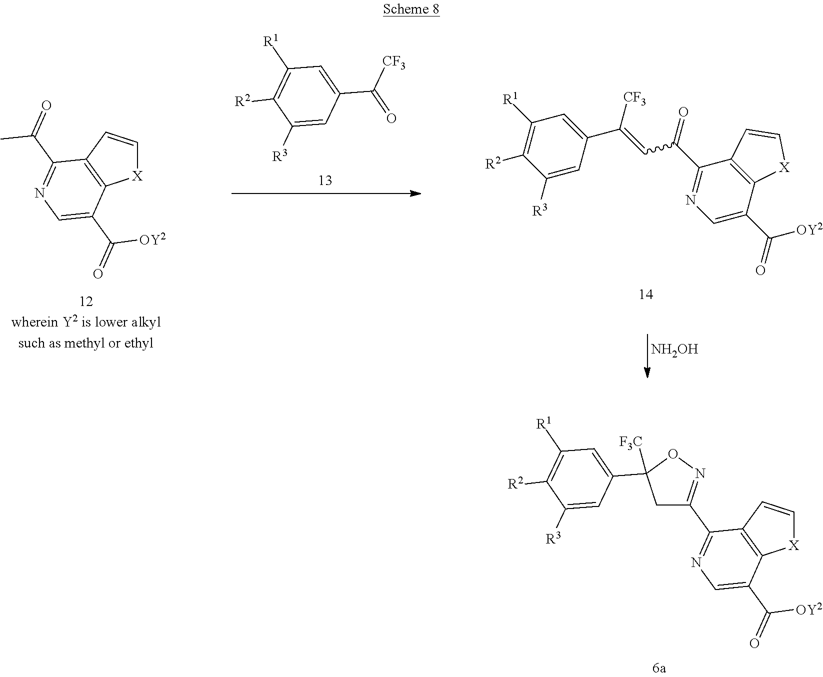

One skilled in the art will recognize that aryl halides of Formula 5a can be prepared analogous to the method described in Scheme 8 above wherein C(═O)OY² is replaced by Y¹.

Ketones of Formula 12 can be prepared by a wide variety of methods known in the art, see, for example, WO 2013/190123, Tetrahedron Letters 2008, 49(42), 6104; and J. Organo-metallic Chemistry 2005, 690(15), 3546, as well as references cited within. As shown in Scheme 9, ketones of Formula 12 can be prepared by reacting a compound of Formula 15 with a vinyl ether such as n-butyl vinyl ether, tributyl(1-ethoxyvinyl)stannane or ethyl vinyl ether, in the presence of a palladium catalyst to provide a coupled product, which is then hydrolyzed with acid to give a compound of Formula 12. Examples of palladium-containing compounds and complexes useful as catalysts in the method of Scheme 9 include PdCl₂(PPh₃)₂(i.e. bis(triphenylphosphine)palladium(II) dichloride), Pd(PPh₃)₄(i.e. tetrakis-(triphenylphosphine)palladium(0)), and [Pd(OAc)₂][Ph₃P(CH₂)₃PPh₃]. Present Example 1 Step E and Example 4 Step D illustrate the method of Scheme 9.

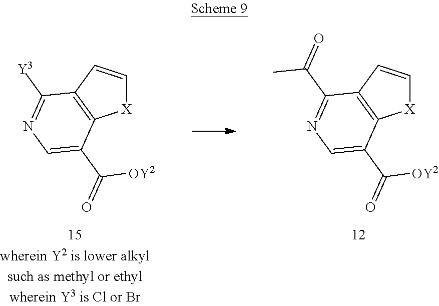

Compounds of Formula 15a (i.e. Formula 15 wherein Y³ is Cl) can be prepared according to general methods known to one skilled in the art in the steps as outlined in Scheme 10. One skilled in the art will recognize that in the first step, methyl ketones of Formula 17 can be prepared analogous to the method described in Scheme 9 above from commercially available bromides of Formula 16. Oxidation of methyl ketones of Formula 17 provides carboxylic acids of Formula 18. Esterification of the carboxylic acids of Formula 18 provides compounds of Formula 15a. Present Example 1 Steps B, C and D illustrate the method of Scheme 10.

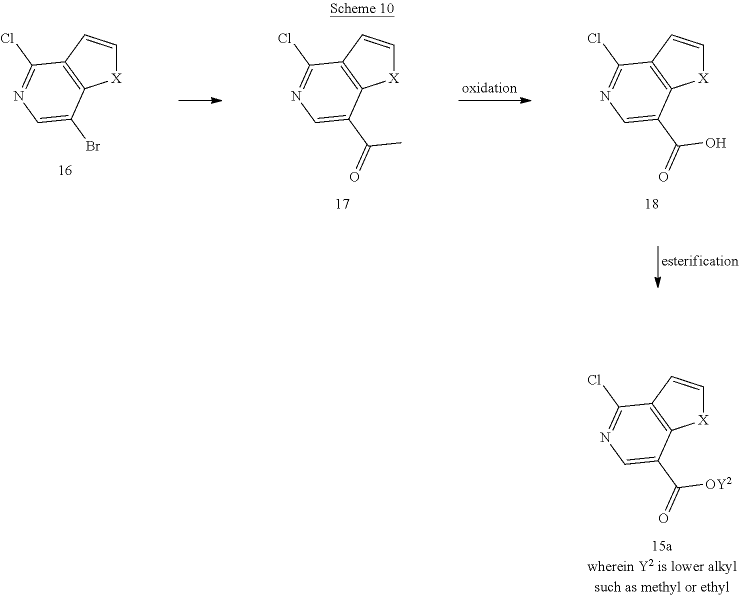

As shown in Scheme 11, compounds of Formula 1b (i.e. Formula 1 wherein T is T-2, J is CH(R⁶)N(R¹³)C(═Z)R¹⁴, R¹³ is H and Z is O) can be prepared by reacting amines of Formula 19 with carboxylic acids of Formula 20 or anhydrides of Formula 21 using a method analogous to Scheme 1. For synthesis of a compound of Formula 1b using an anhydride of Formula 21 see Step E of Example 8.

Alternatively, as also shown in Scheme 11, compounds of Formula 1b (i.e. Formula 1 wherein T is T-2, J is CH(R⁶)N(R¹³)C(═Z)R¹⁴, R¹³ is H and Z is O) can be prepared by reacting amines of Formula 19 with acid chlorides of Formula 22 using a method analogous to Scheme 2.

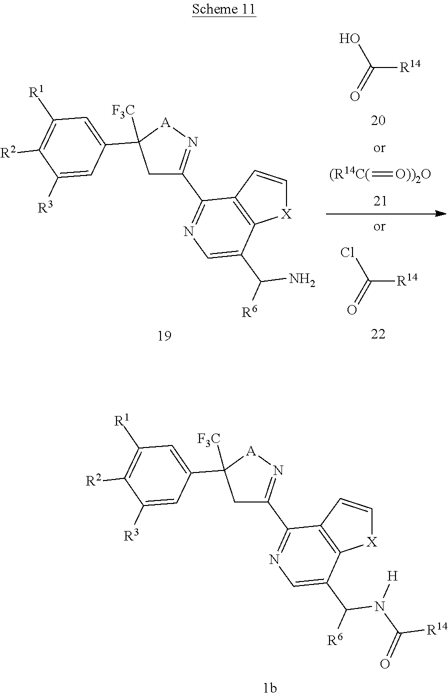

The preparation of compounds of Formula 19a (i.e. Formula 19 wherein R⁶ is H) from compounds of Formula 5 as shown in Scheme 12 is well know in the literature. For example using methods reported in WO 2015/163936; and Journal of Medicinal Chemistry 1978, 21, 613-623, aryl halides of Formula 5, preferably wherein Y¹ is Br or I, can be transformed into nitriles of Formula 23. In a subsequent step, using well known reduction methods, nitriles of Formula 23 can be converted to amines of Formula 19a. See, for example, Journal of Organic Chemistry 2015, 80, 7281-7187; Journal of Medicinal Chemistry 2016, 57, 3215-3230 and the references therein.

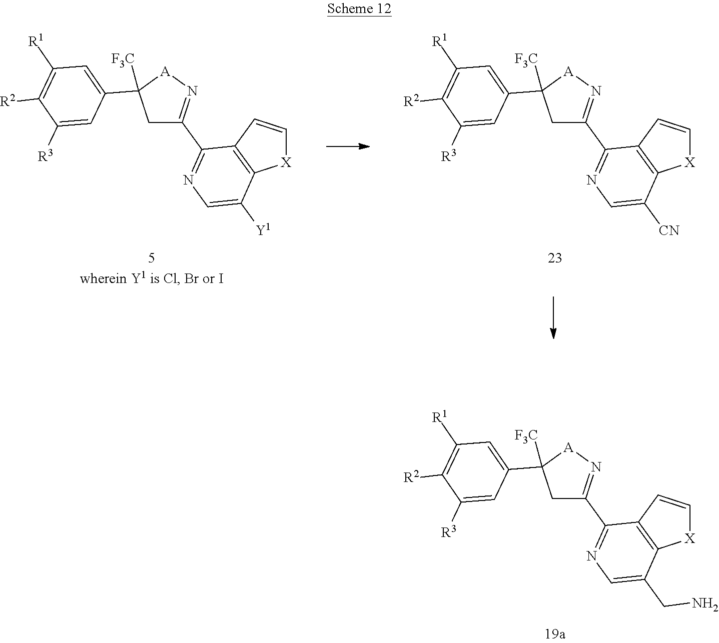

Compounds of Formula 19a can also be prepared according to general methods known to one skilled in the art in the steps as outlined in Scheme 13. In the first step, a suitable amount of a reducing agent such as sodium borohydride, lithium aluminum hydride or diisobutylaluminum hydride, in an aprotic solvent such as dichloromethane or tetrahydrofuran, at a suitable temperature, results in the transformation of esters of Formula 6 to alcohols of Formula 24. Alcohols of Formula 24 can be converted to halides of Formula 25 using reageants such as thionyl chloride, phosphourous oxychloride or carbon tetrabromide. This halogenation reaction is typically conducted in a non-polar solvent such as toluene or dichloromethane. The reduction and halogenation methods of Scheme 13 are illustrated by present Example 8 Step A and B.

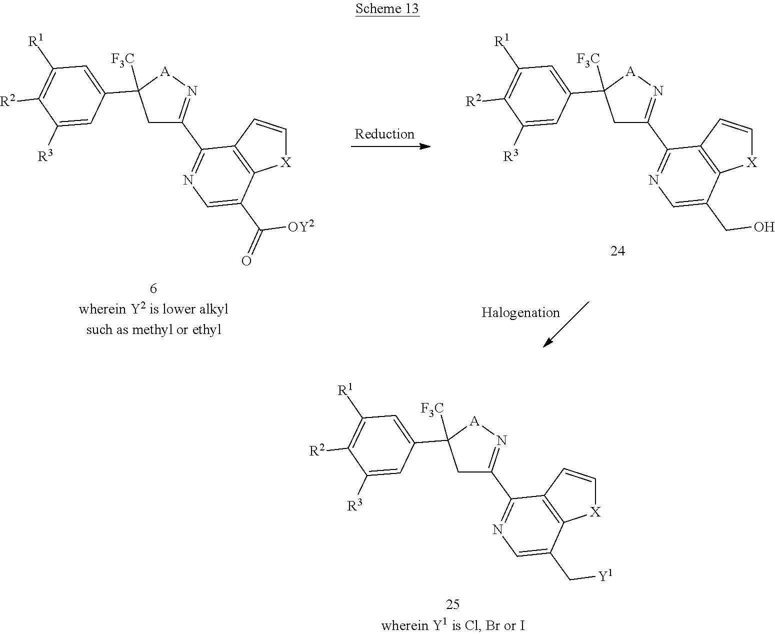

As shown in Scheme 14, compounds of Formula 26 can be prepared by reacting halides of Formula 25 with potassium phthalimide. Compounds of Formula 26 can be converted to amines of Formula 19a, typically in the presence of hydrazine hydrate. See, for example, WO 2009/145816. Also, present Example 8 Steps C and D illustrate the steps of Scheme 14.

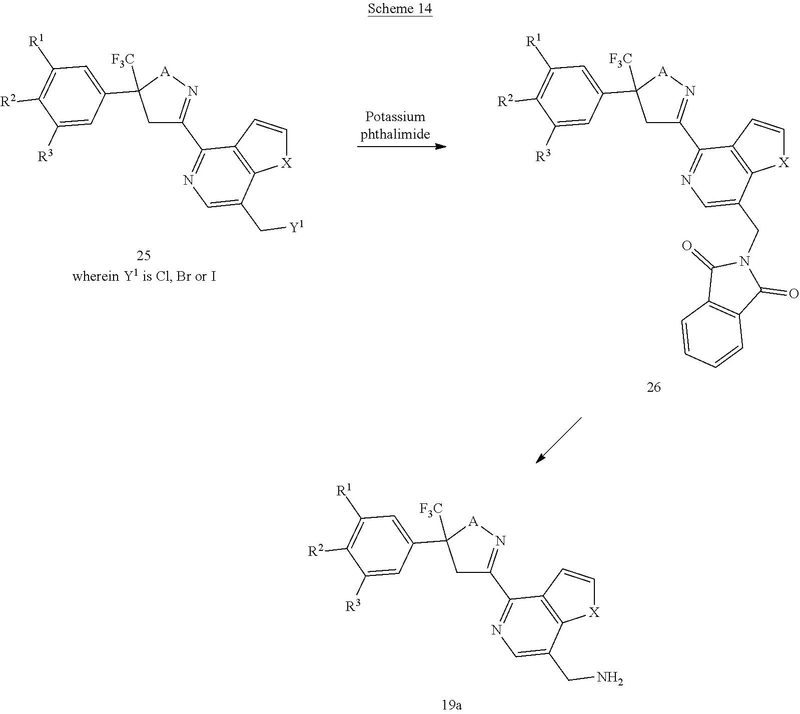

As shown in Scheme 15, compounds of Formula 1c (i.e. Formula 1 wherein T is T-1) can be prepared by methods analogous to those described above in Schemes 1-8 and 11-14 from compounds of Formula 27. Present Example 6 Steps H, I, J and K illustrate the steps of Scheme 15 wherein Y⁴ is methyl, Y⁵ is CO₂Y² and Y² is methyl.

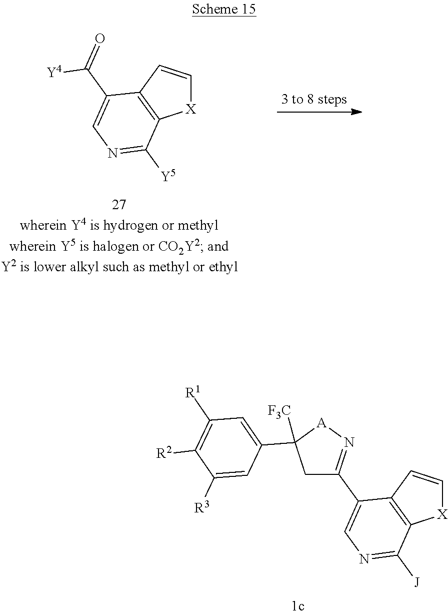

Compounds of Formula 27a (i.e. Formula 27 wherein Y⁴ is methyl and Y⁵ is CO₂Y²) can be prepared according to general methods known to one skilled in the art using the steps as outlined in Scheme 16. General procedures for this sequence of steps are documented in the chemical literature; see for example, WO 2016/037578. Compounds of Formula 29 can be converted to compounds of Formula 28 by treatment with phosphorus chlorides, such as phosphorus oxychloride and phosphorus pentachloride (for conditions, see Example 6, Step E). Methyl ketones of Formula 27b (i.e. Formula 27 wherein Y⁴ is methyl and Y⁵ is Cl) can be prepared analogous to the method described in Scheme 9 above from compounds of Formula 28. Following a method analogous to the carbonylation described in Scheme 6 above a compound of Formula 27b can be converted into a compound of Formula 27a. Compounds of Formula 29 are commercially available and can be prepared according to general methods known to one skilled in the art. Present Example 6 Steps E, F and G illustrate the steps of Scheme 15.

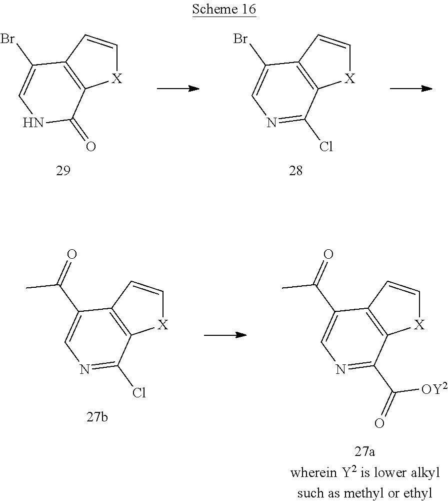

As shown in Scheme 17, compounds of Formula 1d (i.e. Formula 1 wherein T is T-5) can be prepared by methods analogous to those described above in Schemes 1-8 and 11-14 from compounds of Formula 30. Present Example 2 Steps B, C, D and E illustrate the steps of Scheme 17 wherein Y⁴ is methyl, Y⁵ is CO₂Y² and Y² is methyl.

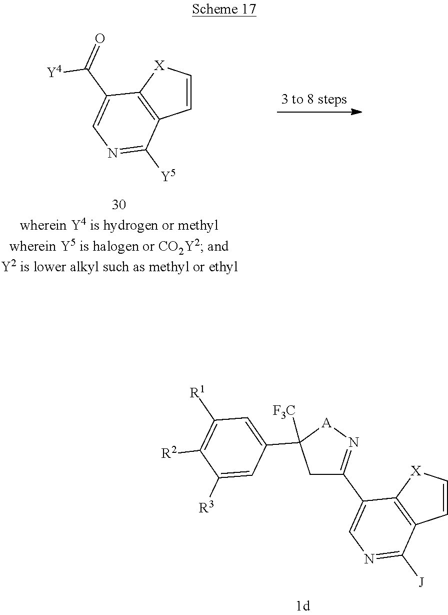

Esters of Formula 30a (i.e. Formula 30 wherein Y⁴ is methyl and Y⁵ is CO₂Y²) can be prepared according to general methods known to one skilled in the art in the steps as outlined in Scheme 18. Methyl ketones of Formula 30b (i.e. Formula 30 wherein Y⁴ is methyl and Y⁵ is Cl) can be prepared analogous to the method described in Scheme 9 above from compounds of Formula 31. Following a method analogous to the carbonylation described in Scheme 6 above compounds of Formula 30b can be converted into esters of Formula 30a. Compounds of Formula 31 are commercially available and can be prepared according to general methods known to one skilled in the art. Present Example 2 Step A illustrates the carbonylation step of Scheme 18.

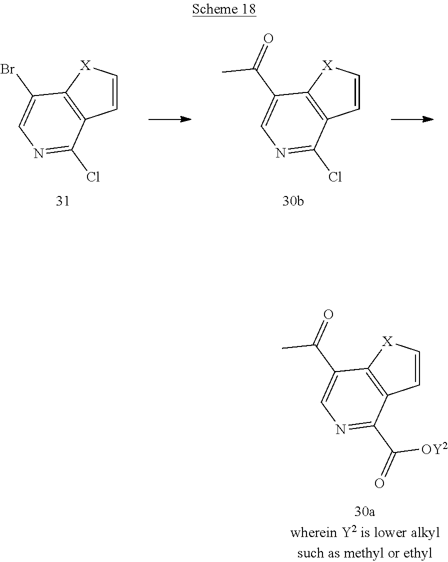

As shown in Scheme 19, compounds of Formula 1e (i.e. Formula 1 wherein T is T-6) can be prepared by methods analogous to those described above in Schemes 1-8 and 11-14 from compounds of Formula 32. Present Example 5 Steps G, H, I and J illustrate the steps of Scheme 19 wherein Y⁴ is methyl, Y⁵ is CO₂Y² and Y² is methyl.

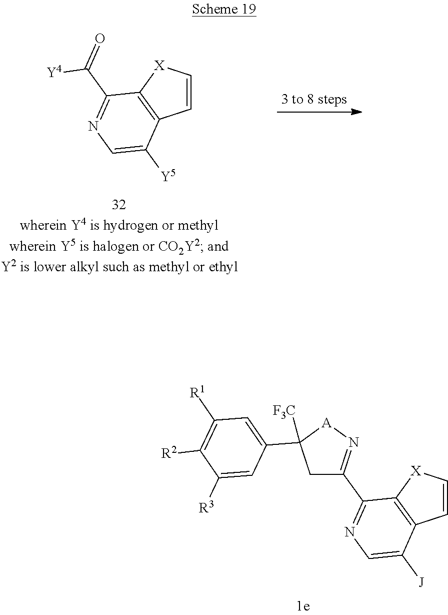

Compounds of Formula 32a (i.e. Formula 32 wherein Y⁴ is methyl and Y⁵ is CO₂Y²) can be prepared according to general methods known to one skilled in the art in the steps as outlined in Scheme 20. Using a method analogous to the carbonylation described in Scheme 6 above compounds of Formula 35 can be converted to esters of Formula 34. In a subsequent step, esters of Formula 34 can be converted to compounds of Formula 33 by treatment with phosphorus halides, such as phosphorus oxychloride and phosphorus pentachloride (for conditions, see Example 5, Step E). Methyl ketones of Formula 32a can be prepared analogous to the method described in Scheme 9 above from compounds of Formula 33. Compounds of Formula 61 are commercially available and can be prepared according to general methods known to one skilled in the art. Present Example 5 Steps D, E and F illustrate the steps of Scheme 20.

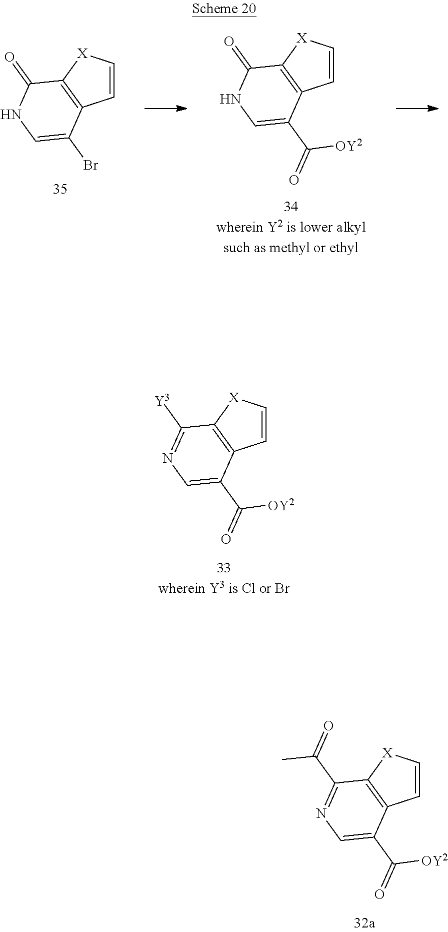

As shown in Scheme 21, compounds of Formula if (i.e. Formula 1 wherein T is T-4) can be prepared by methods analogous to those described above in Schemes 1-8 and 11-14 from compounds of Formula 36. Present Example 3 Steps C, D, E and F illustrate the steps of Scheme 21 wherein Y⁴ is methyl, Y⁵ is CO₂Y² and Y² is methyl.

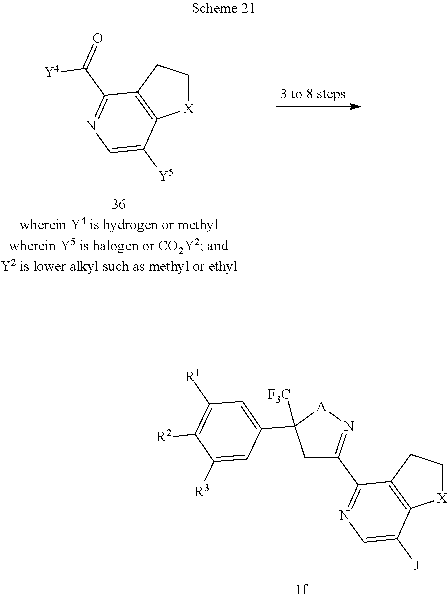

Ketones of Formula 36a (i.e. Formula 36 wherein Y⁴ is methyl and Y⁵ is CO₂Y²) can be prepared according to general methods known to one skilled in the art in the steps as outlined in Scheme 22. Compounds of Formula 12 can be reduced to compounds of Formula 37 by catalytic hydrogenation. Typical conditions involve exposing a compound of Formula 12 to hydrogen gas at a pressure of 70 to 700 kPa in the presence of a metal catalyst such as palladium supported on an inert carrier such as activated carbon, suspended in a solvent such as ethyl acetate at ambient temperature. Also, the method of Scheme 22 is illustrated in Example 3, Step A. One skilled in the art will recognize that other functionalities that may be present in compounds of Formula 12 can also be reduced under catalytic hydrogenation conditions, thus requiring a suitable choice of catalyst and conditions.

In a subsequent step, alcohols of Formula 37 can be oxidized to the corresponding ketones of Formula 36a. Typically mild oxidation conditions involve oxalyl chloride and dimethylsulfoxide in the presence of a base, such as triethylamine (i.e. Swern Oxidation). Further reagents useful for mild oxidations include manganese dioxide, Dess-Martin periodinane, pyridinium chlorochromate or pyridinium dichromate. The method of Scheme 22 using Swern Oxidation conditions is illustrated in Example 3 Step B.

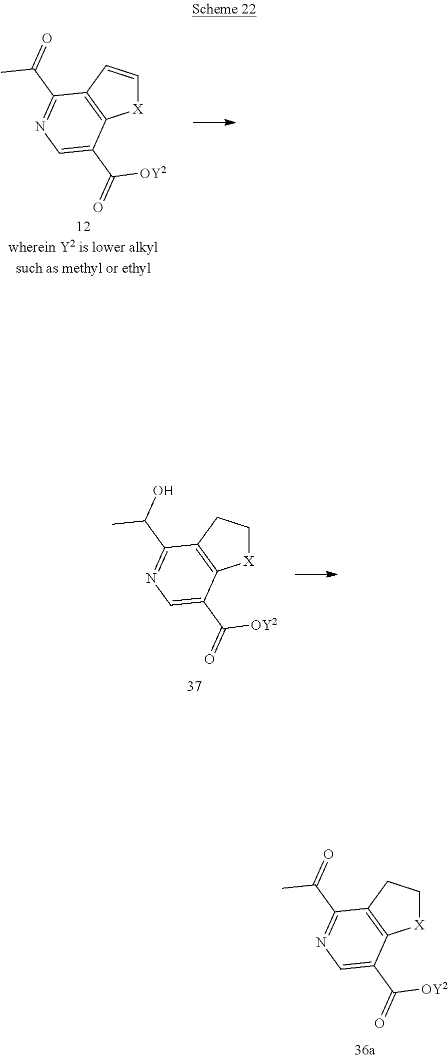

As shown in Scheme 23, compounds of Formula 1g (i.e. Formula 1 wherein T is T-3) can be prepared by methods analogous to those described above in Schemes 1-8 and 11-14 from compounds of Formula 38.

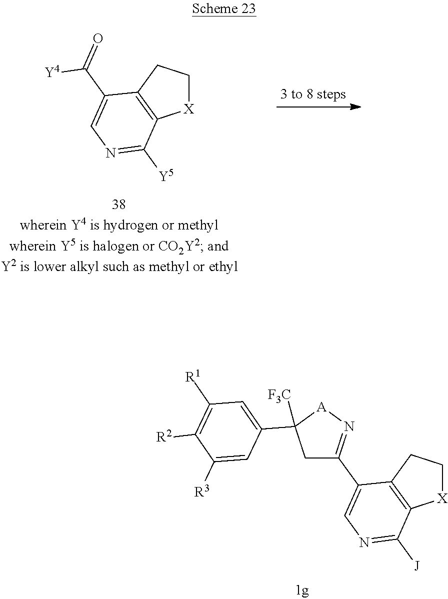

As shown in Scheme 24, one skilled in the art will recognize ketones of Formula 38a (i.e. Formula 38 wherein Y⁴ is methyl and Y⁵ is CO₂Y²) can be prepared by methods analogous to those described above in Scheme 22 from compounds of Formula 27a.

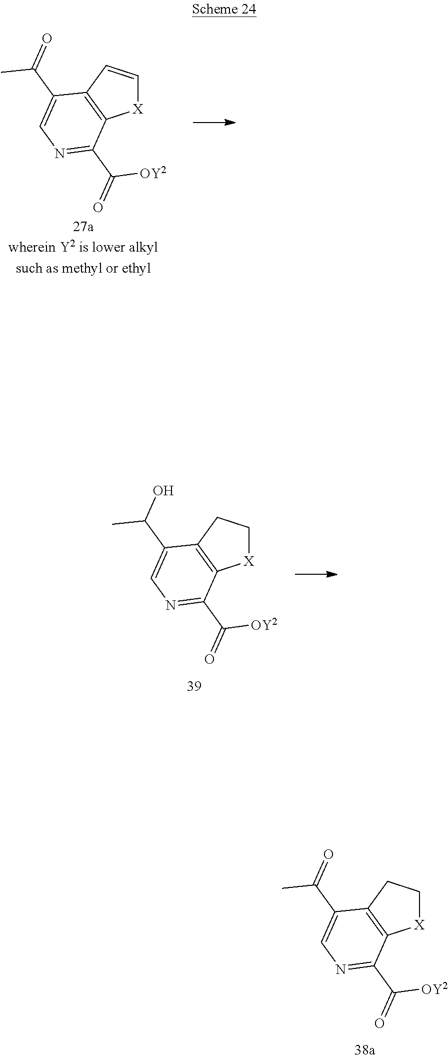

As shown in Scheme 25, compounds of Formula 1h (i.e. Formula 1 wherein T is T-7) can be prepared by methods analogous to those described above in Schemes 1-8 and 11-14 from compounds of Formula 40.

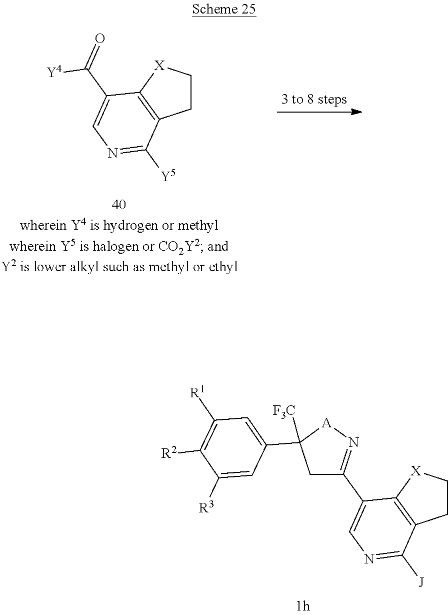

As shown in Scheme 26, one skilled in the art will recognize ketones of Formula 40a (i.e. Formula 40 wherein Y⁴ is methyl and Y⁵ is CO₂Y²) can be prepared by methods analogous to those described above in Scheme 22 from compounds of Formula 30a.

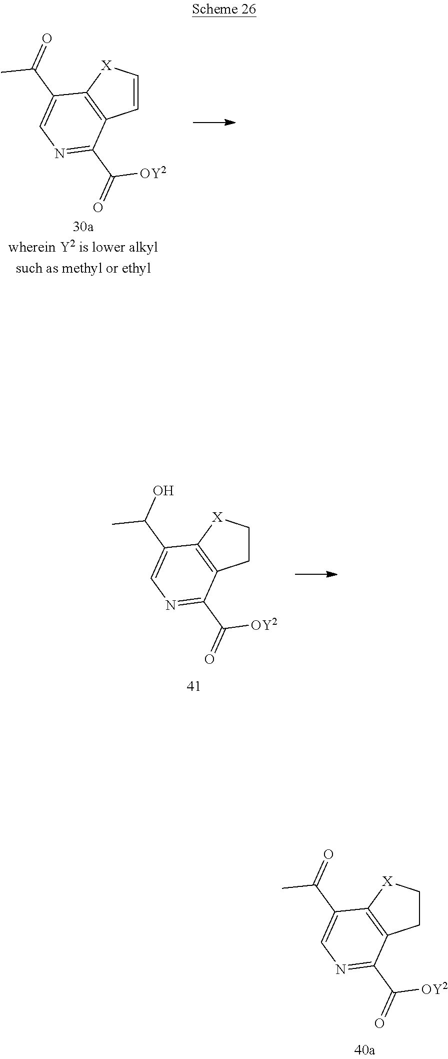

As shown in Scheme 27, compounds of Formula 1i (i.e. Formula 1 wherein T is T-8) can be prepared by methods analogous to those described above in Schemes 1-8 and 11-14 from compounds of Formula 42.

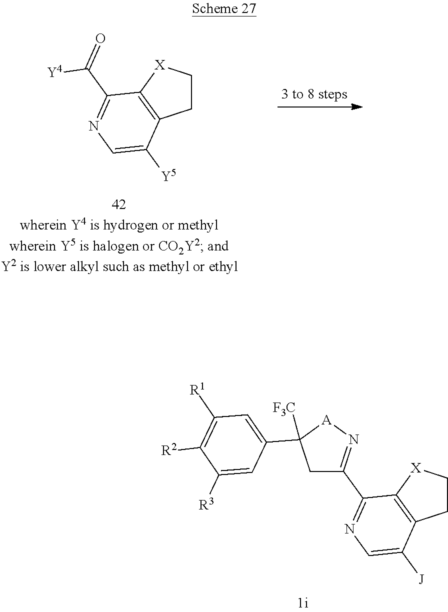

As shown in Scheme 28, one skilled in the art will recognize ketones of Formula 42a (i.e. Formula 42 wherein Y⁴ is methyl and Y⁵ is CO₂Y²) can be prepared by methods analogous to those described above in Scheme 22 from compounds of Formula 32a.

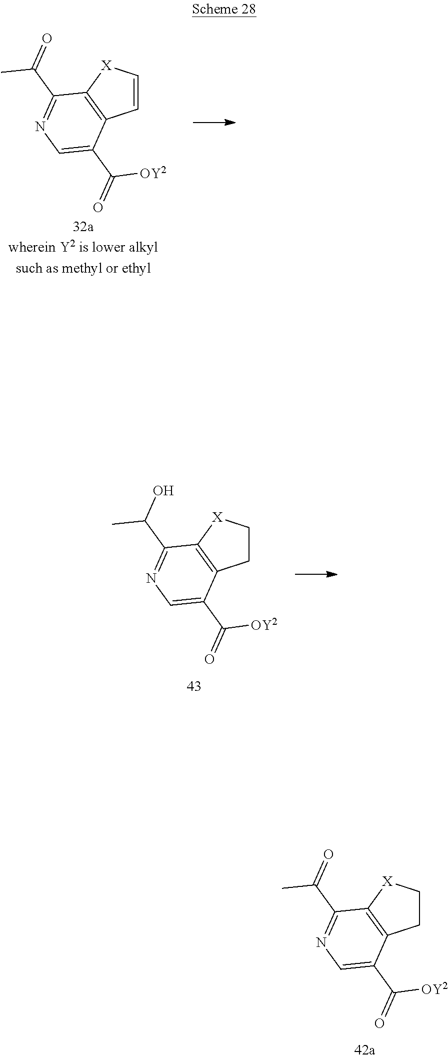

As shown in Scheme 29, compounds of Formula 1j (i.e. Formula 1 wherein T is T-9) can be prepared by methods analogous to those described above in Schemes 1-8 and 11-14 from compounds of Formula 44.

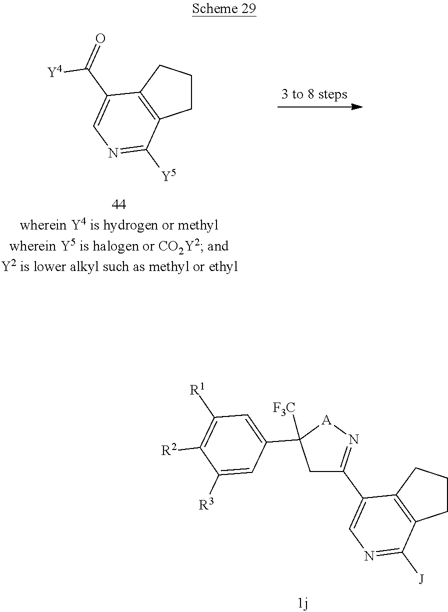

Esters of Formula 44a (i.e. Formula 44 wherein Y⁴ is methyl and Y⁵ is CO₂Y²) can be prepared according to general methods known to one skilled in the art in the steps as outlined in Scheme 30. The methyl ketone of Formula 44b (i.e. Formula 44 wherein Y⁴ is methyl and Y⁵ is Cl) can be prepared analogous to the method described in Scheme 9 above from a compound of Formula 45. Following a method analogous to the carbonylation described in Scheme 6 above a compound of Formula 44b can be converted into esters of Formula 44a. The compound of Formula 45 is commercially available and can be prepared according to general methods known to one skilled in the art.

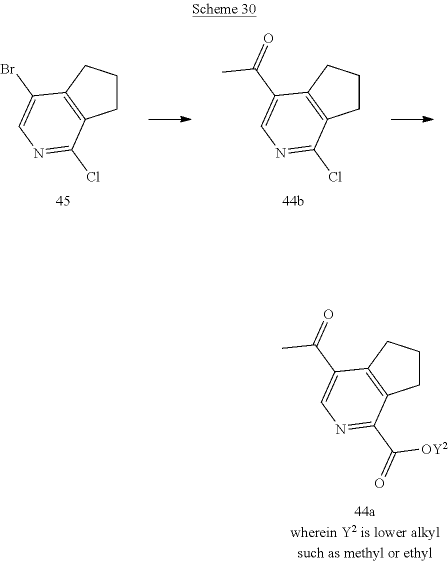

As shown in Scheme 31, compounds of Formula 1k (i.e. Formula 1 wherein T is T-10) can be prepared by methods analogous to those described above in Schemes 1-8 and 11-14 from compounds of Formula 46.

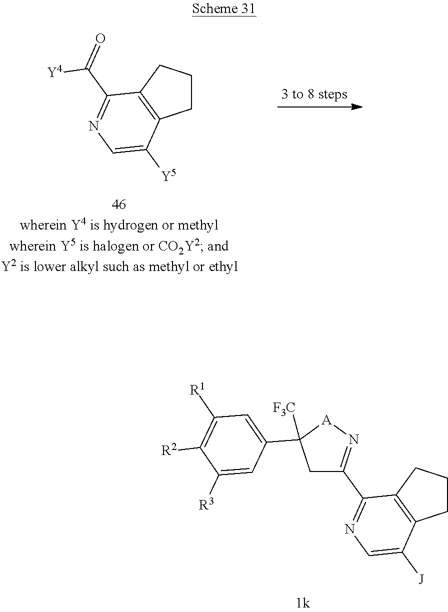

Compounds of Formula 46a (i.e. Formula 46 wherein Y⁴ is methyl and Y⁵ is CO₂Y²) can be prepared according to general methods known to one skilled in the art in the steps as outlined in Scheme 32. Using a method analogous to the carbonylation described in Scheme 6 above a compound of Formula 49 can be converted to esters of Formula 48. Esters of Formula 48 can be converted to compounds of Formula 47 by a method analogous to that described in Scheme 20. Methyl ketones of Formula 46a can be prepared analogous to the method described in Scheme 9 above from compounds of Formula 47. The compounds of Formula 49 is commercially available and can be prepared according to general methods known to one skilled in the art.

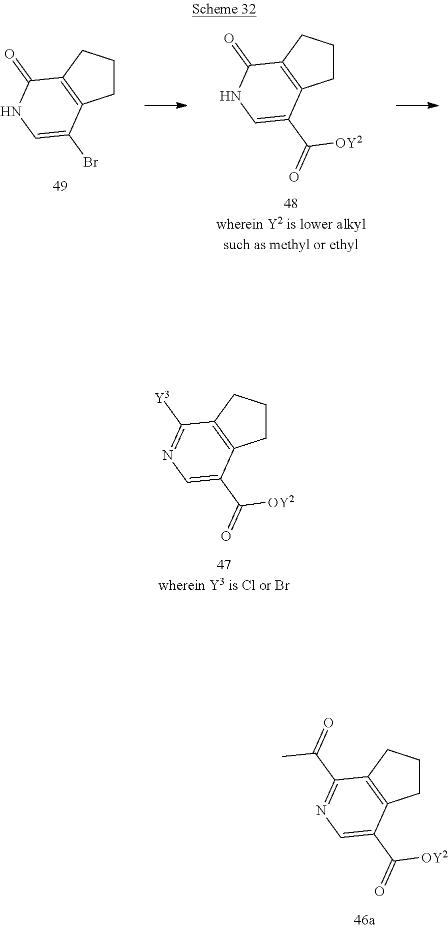

Compounds of Formula 1 prepared by the methods described above wherein Z is O can be converted to the corresponding thioamides wherein Z is S using a variety of standard thiating reagents such as phosphorus pentasulfide or 2,4-bis(4-methoxyphenyl)-1,3-dithia-2,4-diphosphetane-2,4-disulfide (Lawesson's reagent). Reactions of this type are well-known see, for example, Heterocycles 1995, 40, 271-278; J. Med. Chem. 2008, 51, 8124-8134; J. Med. Chem. 1990, 33, 2697-706; Synthesis 1989, (5), 396-3977; J. Chem. Soc., Perkin Trans. 1, 1988, 1663-1668; Tetrahedron 1988 44, 3025-3036; and J. Org. Chem. 1988 53(6), 1323-1326.

Schemes 1 through 32 illustrate methods to prepare compounds of Formula 1 having a variety of substituents. Compounds of Formula 1 having substituents other than those particularly noted for Schemes 1 through 32 can be prepared by general methods known in the art of synthetic organic chemistry, including methods analogous to those described for Schemes 1 to 32.

It is recognized by one skilled in the art that various functional groups can be converted into others to provide different compounds of Formula 1. Compounds of Formula 1, or intermediates for their preparation, may contain aromatic nitro groups, which can be reduced to amino groups, and then converted via reactions well-known in the art (e.g., Sandmeyer reaction) to various halides. By similar known reactions, aromatic amines (anilines) can be converted via diazonium salts to phenols, which can then be alkylated to prepare compounds of Formula 1 with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. Compounds of Formula 1 or precursors thereof containing a halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

Examples of intermediates useful in the preparation of compounds of this invention are shown in Tables I-1 through I-432. The following abbreviations are used in the Tables which follow: Me means methyl, Et means ethyl, i-Pr means iso-propyl, t-Bu means tert-butyl and CN means cyano.

TABLE I-1 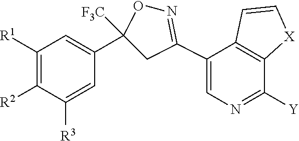
R¹ is Cl, R² is H, R³ is Cl, and X is O.

| Y | Y | Y | Y |
|---|---|---|---|
| Cl | —NO₂ | —CH₂Cl | —CO₂Me |
| Br | —NH₂ | —CH₂Br | —CO₂Et |
| I | —CH₂OH | —CH₂I | —CO₂-i-Pr |
| —CN | —CH₂NH₂ | —CO₂H | —CO₂-t-Bu |

The present disclosure also includes Tables I-2 through I-16, each of which is constructed the same as Table 1 above, except that the row heading in Table 1 (i.e. “R¹ is Cl, R² is H, R³ is Cl and X is O”) is replaced with the respective row headings shown below.

| Table | Row Heading |
|---|---|
| I-2 | R¹ is Cl, R² is H, R³ is CF₃ and X is O. |
| I-3 | R¹ is Cl, R² is F, R³ is CF₃ and X is O. |
| I-4 | R¹ is Cl, R² is F, R³ is Cl and X is O. |
| I-5 | R¹ is H, R² is H, R³ is CF₃ and X is O. |
| I-6 | R¹ is H, R² is F, R³ is CF₃ and X is O. |
| I-7 | R¹ is H, R² is Cl, R³ is CF₃ and X is O. |
| I-8 | R¹ is H, R² is Cl, R³ is Cl and X is O. |
| I-9 | R¹ is Cl, R² is H, R³ is Cl and X is S. |
| I-10 | R¹ is Cl, R² is H, R³ is CF₃ and X is S. |
| I-11 | R¹ is Cl, R² is F, R³ is CF₃ and X is S. |
| I-12 | R¹ is Cl, R² is F, R³ is Cl and X is S. |
| I-13 | R¹ is H, R² is H, R³ is CF₃ and X is S. |
| I-14 | R¹ is H, R² is F, R³ is CF₃ and X is S. |
| I-15 | R¹ is H, R² is Cl, R³ is CF₃ and X is S. |
| I-16 | R¹ is H, R² is Cl, R³ is Cl and X is S. |

TABLE I-17

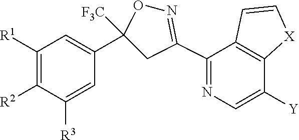

Table I-17 is constructed the same as Table I-1 except that the structure in Table I-1 is replaced with the structure above for Table I-17.

### Table I-18 Through Table I-32

Table I-18 is constructed the same as Table I-2 except that the structure in Table I-2 is replaced with the structure above for Table I-17. Tables I-19 through I-32 are constructed in the same fashion as Tables I-3 through I-16.

TABLE I-33

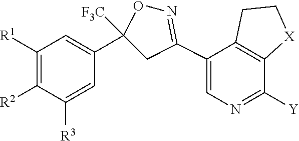

Table I-33 is constructed the same as Table I-1 except that the structure in Table I-1 is replaced with the structure above for Table I-33.

### Table I-34 Through Table I-48

Table I-34 is constructed the same as Table I-2 except that the structure in Table I-2 is replaced with the structure above for Table I-33. Tables I-35 through I-48 are constructed in the same fashion as Tables I-3 through I-16.

TABLE I-49

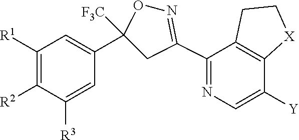

Table I-49 is constructed the same as Table I-1 except that the structure in Table I-1 is replaced with the structure above for Table I-49.

### Table I-50 Through Table I-64

Table I-50 is constructed the same as Table I-2 except that the structure in Table I-2 is replaced with the structure above for Table I-49. Tables I-51 through I-64 are constructed in the same fashion as Tables I-3 through I-16.

TABLE I-65

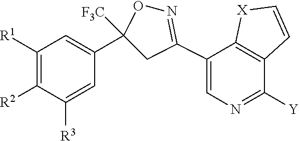

Table I-65 is constructed the same as Table I-1 except that the structure in Table I-1 is replaced with the structure above for Table I-65.

### Table I-66 Through Table I-80

Table I-66 is constructed the same as Table I-2 except that the structure in Table I-2 is replaced with the structure above for Table I-65. Tables I-67 through I-80 are constructed in the same fashion as Tables I-3 through I-16.

TABLE I-81

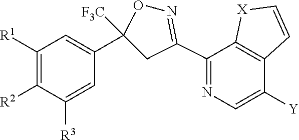

Table I-81 is constructed the same as Table I-1 except that the structure in Table I-1 is replaced with the structure above for Table I-81.

### Table I-82 Through Table I-96

Table I-82 is constructed the same as Table I-2 except that the structure in Table I-2 is replaced with the structure above for Table I-81. Tables I-83 through I-96 are constructed in the same fashion as Tables I-3 through I-16.

TABLE I-97

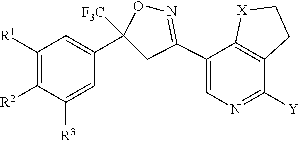

Table I-97 is constructed the same as Table I-1 except that the structure in Table I-1 is replaced with the structure above for Table I-97.

### Table I-98 Through Table I-112

Table I-98 is constructed the same as Table I-2 except that the structure in Table I-2 is replaced with the structure above for Table I-97. Tables I-99 through I-112 are constructed in the same fashion as Tables I-3 through I-16.

TABLE I-113

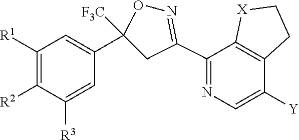

Table I-113 is constructed the same as Table I-1 except that the structure in Table I-1 is replaced with the structure above for Table I-113.

### Table I-114 Through Table I-128

Table I-114 is constructed the same as Table I-2 except that the structure in Table I-2 is replaced with the structure above for Table I-113. Tables I-115 through I-128 are constructed in the same fashion as Tables I-3 through I-16.

TABLE I-129 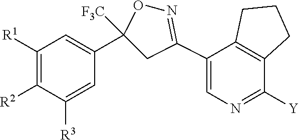
R¹ is Cl, R² is H and R³ is Cl.

| Y | Y | Y | Y |
|---|---|---|---|
| Cl | —NO₂ | —CH₂Cl | —CO₂Me |
| Br | —NH₂ | —CH₂Br | —CO₂Et |
| I | —CH₂OH | —CH₂I | —CO₂-i-Pr |
| —CN | —CH₂NH₂ | —CO₂H | —CO₂-t-Bu |

The present disclosure also includes Tables I-130 through I-136, each of which is constructed the same as Table 129 above, except that the row heading in Table 1 (i.e. “R¹ is Cl, R² is H, R³ is Cl and X is O”) is replaced with the respective row headings shown below.

| Table | Row Heading |
|---|---|
| I-130 | R¹ is Cl, R² is H and R³ is CF₃. |
| I-131 | R¹ is Cl, R² is F and R³ is CF₃. |
| I-132 | R¹ is Cl, R² is F and R³ is Cl. |
| I-133 | R¹ is H, R² is H and R³ is CF₃. |
| I-134 | R¹ is H, R² is F and R³ is CF₃. |
| I-135 | R¹ is H, R² is Cl and R³ is CF₃. |
| I-136 | R¹ is H, R² is Cl and R³ is Cl. |

TABLE I-137

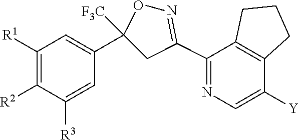

Table I-137 is constructed the same as Table I-129 except that the structure in Table I-129 is replaced with the structure above for Table I-137.

### Table I-138 Through Table I-144

Table I-138 is constructed the same as Table I-130 except that the structure in Table I-130 is replaced with the structure above for Table I-137. Tables I-139 through I-144 are constructed in the same fashion as Tables I-131 through I-136.

TABLE I-145

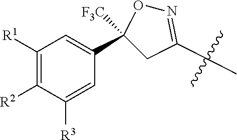

Table I-145 is constructed the same as Table I-1 except that the phenyl isoxazoline fragment of the fused pyridine structure in Table I-1 is replaced with the chiral phenyl isoxazoline fragment above for Table I-145.

### Table I-146 Through Table I-288

Table I-146 is constructed the same as Table I-2 except that the phenyl isoxazoline fragment of the fused pyridine structure in Table I-2 is replaced with the chiral phenyl isoxazoline fragment above for Table I-145. Tables I-147 through I-288 are constructed in the same fashion as Tables I-3 through I-144.

TABLE I-289

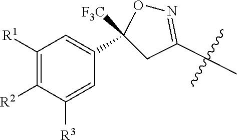

Table I-289 is constructed the same as Table I-1 except that the phenyl isoxazoline fragment of the fused pyridine structure in Table I-1 is replaced with the chiral phenyl isoxazoline fragment above for Table I-289.

### Table I-290 Through Table I-432

Table I-290 is constructed the same as Table I-2 except that the phenyl isoxazoline fragment of the fused pyridine structure in Table I-2 is replaced with the chiral phenyl isoxazoline fragment above for Table I-290. Tables I-291 through I-432 are constructed in the same fashion as Tables I-3 through I-144.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following examples are, therefore, to be construed as merely illustrative, and not limiting of the disclosure in any way whatsoever. Steps in the following examples illustrate a procedure for each step in an overall synthetic transformation, and the starting material for each step may not have necessarily been prepared by a particular preparative run whose procedure is described in other examples or steps. Percentages are by weight except for chromatographic solvent mixtures or where otherwise indicated. Parts and percentages for chromatographic solvent mixtures are by volume unless otherwise indicated. The term “LCMS” means liquid chromatography mass spectrometry. ¹H NMR spectra are reported in ppm downfield from tetramethylsilane; “s” means singlet, “d” means doublet, “t” means triplet, “m” means multiplet, “br s” means broad singlet and “dd” means doublet of doublets. Pressure is defined as the pressure measured relative to the ambient atmospheric pressure. The term “psig” means pounds per square inch gague.

### Example 1

### Preparation of N-cyclopropyl-4-[5-(3,5-dichloro-4-fluorophenyl)-4,5-dihydro-5-(trifluoro-methyl)-3-isoxazolyl]furo[3,2-c]pyridine-7-carboxamide. (Compound 3)

### Step A: Preparation of 7-bromo-4-chlorofuro[3,2-c]pyridine

A mixture of 7-bromofuro[3,2-c]pyridin-4(5H)-one (0.5 g, 4.67 mmol) and phosphorous oxychloride (5 mL) was heated at 120° C. for 1 h. The reaction mixture was concentrated under reduced pressure. The resultant material was diluted with dichloromethane (50 mL), washed with sodium bicarbonate (10% aqueous solution), followed by water and a saturated brine solution. The organic layer was separated, dried over anhydrous sodium sulfate, filtered, concentrated under reduced pressure and purified by silica gel column chromatography (eluting with a gradient of 0 to 100% ethyl acetate in petroleum ether) to yield the title compound (0.45 g).

¹H NMR (CDCl₃, 400 MHz) δ 8.39 (s, 1H), 7.80-7.79 (d, 1H), 6.99-6.98 (d, 1H).

LCMS: m/z: 231.9 [M+H]⁺

### Step B: Preparation of 1-(4-chlorofuro[3,2-c]pyridin-7-yl)ethan-1-one

To a stirred solution of 7-bromo-4-chlorofuro[3,2-c]pyridine (i.e. the product of Step A) (0.45 g, 1.94 mmol) in 1,4-dioxane (10 mL) was added tributyl(1-ethoxy-vinyl)stannane (0.7 g, 1.94 mmol) followed by tetrakis(triphenylphosphine)palladium(0) (0.022 g, 0.019 mmol), heated at 110° C. for 5 h and cooled to room temperature. The reaction mixture was diluted with hydrochloric acid (1.5 N aqueous solution), stirred for 1 h, extracted with dichloromethane (50 mL) and washed with water followed by a saturated brine solution. The organic extract was dried over anhydrous sodium sulfate, filtered, concentrated under reduced pressure and purified by silica gel column chromatography (eluting with a gradient of 0 to 100% ethyl acetate in petroleum ether) to yield the title compound (0.23 g).

¹H NMR (CDCl₃, 400 MHz) δ 8.84 (s, 1H), 7.86-7.85 (d, 1H), 7.03-7.02 (d, 1H), 2.85 (s, 3H).

LCMS: m/z: 196 [M+H]⁺

### Step C: Preparation of 4-chlorofuro[3,2-c]pyridine-7-carboxylic acid

To a solution of 1-(4-chlorofuro[3,2-c]pyridin-7-yl)ethan-1-one (i.e. the product of Step B) (0.23 g, 1.18 mmol) in methanol (5 mL) at 0° C. was added sodium hypochlorite (10% aqueous solution, 2.95 mmol) and stirred at 0° C. for 3 h. The reaction mixture was diluted with water (25 mL), acidified with hydrochloric acid (1.5 N aqueous solution) and extracted with dichloromethane. The organic extract was washed with water followed by a saturated brine solution, dried over anhydrous sodium sulfate, filtered, and concentrated under reduced pressure to yield the title compound (0.15 g).

¹H NMR (CDCl₃, 400 MHz) δ 13.82 (br s, 1H), 8.73 (s, 1H), 8.38-8.37 (d, 1H), 7.23-7.22 (d, 1H).

LCMS: m/z: 197.9 [M+H]⁺

### Step D: Preparation of methyl 4-chlorofuro[3,2-c]pyridine-7-carboxylate

To a solution of 4-chlorofuro[3,2-c]pyridine-7-carboxylic acid (i.e. the product of Step C) (0.15 g, 0.76 mmol) in N,N-dimethylformamide (3 mL) was added potassium carbonate (0.2 g, 1.5 mmol), and methyl iodide (0.16 g, 1.12 mmol) and stirred at room temperature for 2 h. The reaction mixture was diluted with water (10 mL), extracted with ethyl acetate (10 mL), dried over anhydrous sodium sulfate, filtered, concentrated under reduced pressure and purified by silica gel column chromatography (eluting with a gradient of 0 to 100% ethyl acetate in petroleum ether) to yield the title compound (0.15 g).

¹H NMR (CDCl₃, 400 MHz) δ 8.91 (s, 1H), 7.73-7.72 (d, 1H), 6.91-6.90 (d, 1H), 4.07 (s, 3H).

LCMS: m/z: 211.9 [M+H]⁺]⁺

### Step E: Preparation of methyl 4-acetylfuro[3,2-c]pyridine-7-carboxylate

To a solution of methyl 4-chlorofuro[3,2-c]pyridine-7-carboxylate (i.e. the product of Step D) (1.1 g, 5.2 mmol) in 1,4-dioxane (15 mL) was added tributyl(1-ethoxyvinyl)stannane (2.8 g, 7.8 mmol) followed by tetrakis(triphenylphosphine)palladium(0) (0.3 g, 0.26 mmol). The reaction mixture was heated in a sealed tube at 150° C. in a microwave reactor for 1 h. The reaction mixture was cooled to room temperature, diluted with hydrochloric acid (1.5 N aqueous solution), stirred for 1 h, extracted with dichloromethane (50 mL) and washed with water followed by a saturated brine solution. The organic extract was dried over anhydrous sodium sulfate, filtered, concentrated under reduced pressure and purified by silica gel column chromatography (eluting with a gradient of 0 to 100% ethyl acetate in petroleum ether) to yield the title compound (0.70 g).

¹H NMR (CDCl₃, 400 MHz) δ 9.14 (s, 1H), 7.93-7.92 (d, 1H), 7.66-7.65 (d, 1H), 4.09 (s, 3H), 2.85 (s, 3H).

LCMS: m/z: 220.1 [M+H]⁺]⁺

### Step F: Preparation of methyl 4-[(2Z)-3-(3,5-dichloro-4-fluorophenyl)-4,4,4-trifluoro-1-oxo-2-buten-1-yl]furo[3,2-c]pyridine-7-carboxylate

To a solution of methyl 4-acetylfuro[3,2-c]pyridine-7-carboxylate (i.e. the product of Step E) (0.70 g, 3.19 mmol) and 1-(3,5-dichloro-4-fluorophenyl)-2,2,2-trifluoroethan-1-one (1.66 g, 6.38 mmol) in 1,2-dichloroethane (10 mL) was added potassium carbonate (0.65 g, 4.7 mmol) and triethylamine (0.53 g, 5.2 mmol) and heated at 110° C. for 16 h. The reaction mixture was cooled to room temperature, diluted with dichloromethane (50 mL) and washed with water followed by a saturated brine solution. The organic layer was separated, dried over anhydrous sodium sulfate, filtered, concentrated under reduced pressure and purified by silica gel column chromatography (eluting with a gradient of 0 to 100% ethyl acetate in petroleum ether) to yield the title compound (0.70 g).

¹H NMR (CDCl₃, 400 MHz) δ 9.18 (s, 1H), 7.94-7.93 (d, 1H), 7.53-7.52 (d, 1H), 7.32-7.31 (d, 2H), 4.11 (s, 3H).

LCMS: m/z: 462 [M+H]⁺

### Step G: Preparation of methyl 4-[5-(3,5-dichloro-4-fluorophenyl)-4,5-dihydro-5-(trifluoromethyl)-3-isoxazolyl]furo[3,2-c]pyridine-7-carboxylate

To a solution of methyl 4-[(2Z)-3-(3,5-dichloro-4-fluorophenyl)-4,4,4-trifluoro-1-oxo-2-buten-1-yl]furo[3,2-c]pyridine-7-carboxylate (i.e. the product of Step F) (0.7 g, 1.51 mmol) in 1,2-dimethoxyethane (20 mL) at 0° C. was added hydroxylamine hydrochloride (0.21 g, 3.03 mmol) and lithium hydroxide monohydrate (0.127 g, 3.03 mmol) and allowed to warm to room temperature over 3 h. The reaction mixture was diluted with dichloromethane (50 mL) and washed with water followed by a saturated brine solution. The organic layer was separated, dried over anhydrous sodium sulfate, filtered, concentrated under reduced pressure and purified by silica gel column chromatography (eluting with a gradient of 0 to 100% ethyl acetate in petroleum ether) to yield the title compound (0.18 g). ¹H NMR (CDCl₃, 400 MHz) δ 9.08 (s, 1H), 7.91 (s, 1H), 7.65-7.63 (d, 2H), 7.51-7.50 (d, 1H), 4.44-4.40 (d, 1H), 4.08 (s, 3H), 4.08-4.04 (d, 1H).

LCMS: m/z: 476.9 [M+H]⁺

### Step H: Preparation of 4-[5-(3,5-dichloro-4-fluorophenyl)-4,5-dihydro-5-(trifluoro-methyl)-3-isoxazolyl]furo[3,2-c]pyridine-7-carboxylic acid

To a solution of methyl 4-[5-(3,5-dichloro-4-fluorophenyl)-4,5-dihydro-5-(trifluoro-methyl)-3-isoxazolyl]furo[3,2-c]pyridine-7-carboxylate (i.e. the product of Step G) (0.18 g, 0.377 mmol) in a mixture of methanol (3 mL), tetrahydrofuran (3 mL) and water (1 mL) at 0° C. was added lithium hydroxide monohydrate (0.032 g, 0.755 mmol) and allowed to warm to room temperature over a period of 5 h. The reaction mixture was acidified with hydrochloric acid (1.5 N aqueous solution), diluted with dichloromethane (50 mL), and washed with water followed by a saturated brine solution. The organic layer was separated, dried over anhydrous sodium sulfate, filtered, and concentrated under reduced pressure to yield the title compound (0.17 g).

¹H NMR (CDCl₃, 400 MHz) δ 9.08 (s, 1H), 7.91 (s, 1H), 7.65-7.63 (d, 2H), 7.51-7.50 (d, 1H), 4.44-4.40 (d, 1H), 4.08-4.04 (d, 1H).

LCMS: m/z: 463 [M+H]⁺

### Step I: Preparation of N-cyclopropyl-4-[5-(3,5-dichloro-4-fluorophenyl)-4,5-dihydro-5-(trifluoromethyl)-3-isoxazolyl]furo[3,2-c]pyridine-7-carboxamide. (Compound 3)

To a stirred solution of 4-[5-(3,5-dichloro-4-fluorophenyl)-4,5-dihydro-5-(trifluoro-methyl)-3-isoxazolyl]furo[3,2-c]pyridine-7-carboxylic acid (i.e. the product of Step H) (0.06 g, 0.129 mmol), cyclopropylamine (0.014 g, 0.259 mmol) and triethylamine (0.032 g, 0.32 mmol) in ethyl acetate (5 mL) at 0° C. was added propylphosphonic anhydride (50% solution in ethyl acetate) (0.123 g 0.195 mmol) and stirred at room temperature for 16 h. The reaction mixture was diluted with ethyl acetate (20 mL) and washed with water followed by a saturated brine solution. The organic layer was separated, dried over anhydrous sodium sulfate, filtered, concentrated under reduced pressure and purified by silica gel column chromatography (eluting v with a gradient of 0 to 100% ethyl acetate in petroleum ether) to yield the title compound, a compound of the present invention, as a solid (0.05 g).

¹H NMR (CDCl₃, 400 MHz) δ 9.22 (s, 1H), 7.87-7.86 (d, 1H), 7.64-7.63 (d, 2H), 7.55-7.54 (d, 1H), 7.34 (s, 3H), 4.44-4.40 (m, 1H), 4.07-4.02 (m, 1H), 3.05-3.03 (m, 1H), 0.99-0.94 (q, 2H), 0.77-0.74 (q, 2H).

LCMS: m/z: 501.9 [M+H]⁺

### Example 2

### Preparation of N-cyclopropyl-7-[5-(3,5-dichloro-4-fluorophenyl)-4,5-dihydro-5-(tri-fluoromethyl)-3-isoxazolyl]furo[3,2-c]pyridine-4-carboxamide. (Compound 21)

### Step A: Preparation of methyl 7-acetylfuro[3,2-c]pyridine-4-carboxylate

To a solution of 1-(4-chlorofuro[3,2-c]pyridin-7-yl)ethan-1-one (1.0 g, 5.12 mmol) in methanol (30 mL) was added sodium acetate (0.84 g, 10.2 mmol) and dichloro[1,1′-bis(diphenylphosphino)ferrocene]palladium(II) dichloromethane complex (0.33 g, 0.41 mmol). The reaction mixture was sealed in a pressure reactor, was subjected to 14 psig of carbon monoxide, heated at 70° C., and agitated for 16 h. The reaction mixture was cooled to room temperature, diluted with ethyl acetate (100 mL) and washed with water followed by a saturated brine solution. The organic extract was dried over anhydrous sodium sulfate, filtered, concentrated under reduced pressure, and purified by silica gel column chromatography (eluting with a gradient of 0 to 100% ethyl acetate in petroleum ether) to yield the title compound (0.52 g).

¹H NMR (CDCl₃, 400 MHz) δ 9.12 (s, 1H), 7.93-7.92 (d, 1H), 7.59-7.58 (d, 1H), 4.12 (s, 3H), 2.91 (s, 3H).

LCMS: m/z: 219.9 [M+H]⁺

### Step B: Preparation of methyl 7-[(2Z)-3-(3,5-dichloro-4-fluorophenyl)-4,4,4-trifluoro-1-oxo-2-buten-1-yl]furo[3,2-c]pyridine-4-carboxylate

To a stirred solution of methyl 7-acetylfuro[3,2-c]pyridine-4-carboxylate (i.e. the product of Step A) (0.7 g, 3.19 mmol) and 1-(3,5-dichloro-4-fluorophenyl)-2,2,2-tri-fluoroethan-1-one (1.66 g, 6.39 mmol) in 1,2-dichloroethane (15 mL) was added potassium carbonate (0.66 g, 4.78 mmol) and triethylamine (0.482 g, 4.78 mmol). The reaction mixture was heated at 110° C. for 16 h, cooled to room temperature, diluted with dichloromethane (50 mL), and washed with water followed by a saturated brine solution. The organic extract was dried over anhydrous sodium sulfate, filtered, concentrated under reduced pressure, and purified by silica gel column chromatography (eluting with a gradient of 0 to 100% ethyl acetate in petroleum ether) to yield the title compound (0.70 g).

¹H NMR (CDCl₃, 400 MHz) δ 8.99 (s, 1H), 7.97-7.96 (d, 1H), 7.75-7.74 (d, 1H), 7.61 (d, 1H), 7.28-7.26 (d, 2H), 4.12 (s, 3H).

LCMS: m/z: 462 [M+H]⁺

### Step C: Preparation of methyl 7-[5-(3,5-dichloro-4-fluorophenyl)-4,5-dihydro-5-(trifluoromethyl)-3-isoxazolyl]furo[3,2-c]pyridine-4-carboxylate

To a stirred solution of methyl 7-[(2Z)-3-(3,5-dichloro-4-fluorophenyl)-4,4,4-tri-fluoro-1-oxo-2-buten-1-yl]furo[3,2-c]pyridine-4-carboxylate (i.e. the product of Step B) (0.65 g, 1.4 mmol) in dichloromethane (20 mL) at 0° C. was added hydroxylamine hydrochloride (0.24 g, 3.5 mmol) and 1,8-diazabicyclo[5.4.0]undec-7-ene (0.425 g, 2.8 mmol). The reaction mixture was allowed to warm to room temperature over a period of 5 h. The reaction mixture was diluted with dichloromethane (50 mL), washed with water followed by a saturated brine solution, dried over anhydrous sodium sulfate, filtered, concentrated under reduced pressure and purified by silica gel column chromatography (eluting with a gradient of 0 to 100% ethyl acetate in petroleum ether) to yield the title compound (0.55 g).

¹H NMR (CDCl₃, 400 MHz) δ 9.04 (s, 1H), 7.91 (s, 1H), 7.66-7.64 (d, 2H), 7.57 (s, 1H), 4.44-4.40 (d, 1H), 4.12 (s, 3H), 4.01-3.98 (d, 1H).

LCMS: m/z: 476.8 [M+H]⁺

### Step D: Preparation of 7-[5-(3,5-dichloro-4-fluorophenyl)-4,5-dihydro-5-(trifluoro-methyl)-3-isoxazolyl]furo[3,2-c]pyridine-4-carboxylic acid

To a stirred solution of methyl 7-[5-(3,5-dichloro-4-fluorophenyl)-4,5-dihydro-5-(trifluoromethyl)-3-isoxazolyl]furo[3,2-c]pyridine-4-carboxylate (i.e. the product of Step C) (0.5 g, 1.05 mmol) in tetrahydrofuran (10 mL) and water (1 mL) at 0° C. was added lithium hydroxide monohydrate (0.13 g, 3.14 mmol) and allowed to warm to room temperature over 6 h. The reaction mixture was acidified with hydrochloric acid (1.5 N aqueous solution), diluted with dichloromethane (50 mL), washed with water followed by a saturated brine solution, dried over anhydrous sodium sulfate, filtered, and concentrated under reduced pressure to yield the title compound (0.43 g).

¹H NMR (DMSO-d₆, 400 MHz) δ 13.66 (br s, 1H), 8.84 (s, 1H), 8.41 (s, 1H), 7.88 (s, 2H), 7.48-7.46 (s, 1H), 4.65-4.48 (dd, 2H).

LCMS: m/z: 462.8 [M+H]⁺

### Step E: Preparation of N-cyclopropyl-7-[5-(3,5-dichloro-4-fluorophenyl)-4,5-dihydro-5-(trifluoromethyl)-3-isoxazolyl]furo[3,2-c]pyridine-4-carboxamide. (Compound 21)

To a stirred solution of 7-[5-(3,5-dichloro-4-fluorophenyl)-4,5-dihydro-5-(trifluoro-methyl)-3-isoxazolyl]furo[3,2-c]pyridine-4-carboxylic acid (i.e. the product of Step D) (0.12 g, 0.259 mmol) in ethyl acetate (5 mL) was added cyclopropylamine (0.029 g, 0.52 mmol) and triethylamine (0.065 g, 0.647 mmol), and cooled to 0° C. Propylphosphonic anhydride (50% solution in ethyl acetate) (0.247 g 0.388 mmol) was added to the reaction mixture and stirred at room temperature for 16 h. The reaction mixture was then diluted with ethyl acetate (20 mL) and washed with water followed by a saturated brine solution. The organic layer was dried over anhydrous sodium sulfate, filtered, and concentrated under reduced pressure and purified by silica gel column chromatography (eluting with a gradient of 0 to 100% ethyl acetate in petroleum ether) to yield the title compound, a compound of the present invention, as a solid (0.10 g).

¹H NMR (CDCl₃, 400 MHz) δ 8.85 (s, 1H), 8.15 (s, 1H), 7.87 (s, 1H), 7.79 (s, 1H), 7.64 (s, 1H), 4.44-4.40 (d, 1H), 4.00-3.99 (d, 1H), 3.0-2.95 (m, 1H), 0.95-0.92 (t, 2H), 0.76-0.74 (t, 2H).

### Example 3

### Preparation of N-cyclopropyl-4-[5-(3,5-dichloro-4-fluorophenyl)-4,5-dihydro-5-(trifluoromethyl)-3-isoxazolyl]-2,3-dihydrofuro[3,2-c]pyridine-7-carboxamide. (Compound 17)

### Step A: Preparation of methyl 4-(1-hydroxyethyl)-2,3-dihydrofuro[3,2-c]pyridine-7-carboxylate

To a solution of methyl 4-acetylfuro[3,2-c]pyridine-7-carboxylate (i.e. the product of Example 1 Step E) (1.4 g, 6.39 mmol) in ethyl acetate (30 mL) was added palladium (10% on carbon, 0.7 g). The reaction mixture was sealed in a pressure reactor, subjected to 72 psig of hydrogen, agitated for 72 h and filtered through Celite® diatomaceous earth filter aid, rinsing with ethyl acetate. The filtrate was concentrated under reduced pressure and purified by silica gel column chromatography (eluting with a gradient of 0 to 100% ethyl acetate in petroleum ether) to yield the title compound (1.1 g).

LCMS: m/z: 224 [M+H]⁺

### Step B: Preparation of methyl 4-acetyl-2,3-dihydrofuro[3,2-c]pyridine-7-carboxylate

To a stirred solution of dimethyl sulfoxide (1.9 g, 24.4 mmol) in dichloromethane (10 mL) was added oxalyl chloride (2.05 g, 26.3 mmol) at −78° C. and stirred 1 h. A solution of methyl 4-(1-hydroxyethyl)-2,3-dihydrofuro[3,2-c]pyridine-7-carboxylate (i.e. the product of Step A) (1.0 g, 4.5 mmol) in dichloromethane (10 mL) was added dropwise to reaction mixture and allowed to warm to room temperature over 4 h. The reaction mixture was quenched by a dropwise addition of triethylamine (4.1 g, 40.7 mmol). The reaction mixture was diluted with dichloromethane (20 mL), washed with water followed by a saturated brine solution, dried over anhydrous sodium sulfate, filtered, concentrated under reduced pressure and purified by silica gel column chromatography (eluting with a gradient of 0 to 100% ethyl acetate in petroleum ether) to yield the title compound (0.40 g).

¹H NMR (CDCl₃, 400 MHz) δ 8.95 (s, 1H), 4.90-4.85 (t, 2H), 3.98 (s, 3H), 3.64-3.60 (t, 2H), 2.74 (s, 3H).

LCMS: m/z: 222.1 [M+H]⁺

### Step C: Preparation of methyl 4-[(2Z)-3-(3,5-dichloro-4-fluorophenyl)-4,4,4-trifluoro-1-oxo-2-buten-1-yl]-2,3-dihydrofuro[3,2-c]pyridine-7-carboxylate

To a stirred solution of methyl 4-acetyl-2,3-dihydrofuro[3,2-c]pyridine-7-carboxylate (i.e. the product of Step B) (0.4 g, 1.79 mmol) and 1-(3,5-dichloro-4-fluorophenyl)-2,2,2-trifluoroethan-1-one (0.92 g, 3.58 mmol) in 1,2-dichloroethane (10 mL) was added potassium carbonate (0.37 g, 2.68 mmol) and triethylamine (0.27 g, 2.68 mmol). The reaction mixture was heated at 110° C. for 16 h, cooled to room temperature, diluted with dichloromethane (50 mL) and washed with water followed by a saturated brine solution. The organic extract was dried over anhydrous sodium sulfate, filtered, concentrated under reduced pressure, and purified by silica gel column chromatography (eluting with a gradient of 0 to 100% ethyl acetate in petroleum ether) to yield the title compound (0.40 g).

¹H NMR (CDCl₃, 400 MHz) δ 8.96 (s, 1H), 8.17 (s, 1H), 7.60-7.58 (d, 1H), 7.27-7.26 (d, 1H), 4.89-4.84 (t, 2H), 3.99 (s, 3H), 3.53-3.49 (t, 2H).

LCMS: m/z: 463.9 [M+H]⁺

### Step D: Preparation of methyl 4-[5-(3,5-dichloro-4-fluorophenyl)-4,5-dihydro-5-(trifluoromethyl)-3-isoxazolyl]-2,3-dihydrofuro[3,2-c]pyridine-7-carboxylate

To a stirred solution of methyl 4-[(2Z)-3-(3,5-dichloro-4-fluorophenyl)-4,4,4-trifluoro-1-oxo-2-buten-1-yl]-2,3-dihydrofuro[3,2-c]pyridine-7-carboxylate (i.e. the product of Step C) (0.4 g, 0.86 mmol) in dichloromethane (10 mL) at 0° C. was added hydroxylamine hydrochloride (0.15 g, 2.16 mmol) and 1,8-diazabicyclo[5.4.0]undec-7-ene (0.26 g, 1.72 mmol). The reaction mixture was allowed to warm to room temperature over a period of 5 h. The reaction mixture was diluted with dichloromethane (50 mL), washed with water followed by a saturated brine solution, dried over anhydrous sodium sulfate, filtered, concentrated under reduced pressure and purified by silica gel column chromatography (eluting with a gradient of 0 to 100% ethyl acetate in petroleum ether) to yield the title compound (0.30 g).

¹H NMR (CDCl₃, 400 MHz) δ 8.88 (s, 1H), 7.61-7.59 (d, 2H), 4.92-4.88 (t, 2H), 4.34-4.30 (d, 1H), 3.97 (s, 3H), 3.97-3.94 (d, 1H), 3.57-3.50 (t, 2H).

LCMS: m/z: 478.7 [M+H]⁺

### Step E: Preparation of 4-[5-(3,5-dichloro-4-fluorophenyl)-4,5-dihydro-5-(trifluoro-methyl)-3-isoxazolyl]-2,3-dihydrofuro[3,2-c]pyridine-7-carboxylic acid

To a stirred solution of methyl 4-[(2Z)-3-(3,5-dichloro-4-fluorophenyl)-4,4,4-trifluoro-1-oxo-2-buten-1-yl]-2,3-dihydrofuro[3,2-c]pyridine-7-carboxylate (i.e. the product of Step D) (0.3 g, 0.645 mmol) in tetrahydrofuran (10 mL) and water (1 mL) at 0° C. was added lithium hydroxide monohydrate (0.073 g, 1.73 mmol) and allowed to warm to room temperature over 6 h. The reaction mixture was acidified with hydrochloric acid (1.5 N aqueous solution), diluted with dichloromethane (50 mL), washed with water followed by a saturated brine solution, dried over anhydrous sodium sulfate, filtered, and concentrated under reduced pressure to yield the title compound (0.25 g).

¹H NMR (DMSO-d₆, 400 MHz) δ 13.32 (s, 1H), 8.74 (s, 1H), 7.91 (s, 2H), 4.83-4.79 (t, 2H), 4.34-4.32 (m, 2H), 3.48-3.43 (t, 2H).

LCMS: m/z: 464.7 [M+H]⁺

### Step F: Preparation of N-cyclopropyl-4-[5-(3,5-dichloro-4-fluorophenyl)-4,5-dihydro-5-(trifluoromethyl)-3-isoxazolyl]-2,3-dihydrofuro[3,2-c]-pyridine-7-carboxamide. (Compound 17)

To a stirred solution of 4-[5-(3,5-dichloro-4-fluorophenyl)-4,5-dihydro-5-(trifluoro-methyl)-3-isoxazolyl]-2,3-dihydrofuro[3,2-c]pyridine-7-carboxylic acid (i.e., the product of Step E) (0.08 g, 0.172 mmol) in ethyl acetate (5 mL) was added cyclopropylamine (0.019 g, 0.34 mmol) and triethylamine (0.043 g, 0.43 mmol), and cooled to 0° C. Propylphosphonic anhydride (50% solution in ethyl acetate) (0.164 g 0.258 mmol) was added to the reaction mixture and stirred at room temperature for 16 h. The reaction mixture was then diluted with ethyl acetate (20 mL) and washed with water followed by a saturated brine solution. The organic layer was dried over anhydrous sodium sulfate, filtered, concentrated under reduced pressure and purified by silica gel column chromatography (eluting with a gradient of 0 to 100% ethyl acetate in petroleum ether) to yield the title compound, a compound of the present invention, as a solid (0.07 g).

¹H NMR (CDCl₃, 400 MHz) δ 9.07 (s, 1H), 7.60-7.59 (d, 2H), 7.36 (s, 1H), 4.92-4.87 (t, 2H), 4.35-4.30 (d, 1H), 3.98-3.93 (d, 1H), 3.60-3.54 (m, 2H), 2.97-2.93 (m, 1H), 0.91-0.88 (q, 2H), 0.77-0.66 (q, 2H).

LCMS: m/z: 503.9 [M+H]⁺

### Example 4

### Preparation of 4-[5-(3,5-dichlorophenyl)-4,5-dihydro-5-(trifluoromethyl)-3-isoxazolyl]-N-3-thietanylthieno[3,2-c]pyridine-7-carboxamide. (Compound 27)

### Step A: Preparation of 7-bromothieno[3,2-c]pyridin-4(5H)-one

To a stirred solution of thieno[3,2-c]pyridin-4(5H)-one (15.0 g, 98.7 mmol) in N,N-dimethylformamide (150 mL) at 0° C. was added N-bromosuccinimide (17.6 g, 98.7 mmol) portionwise and stirred at room temperature for 4 h. The reaction mixture was poured over ice water (300 mL), filtered and washed with water (200 mL). The solid collected was dried under reduced pressure to provide the title compound as an off-white solid (18.0 g).

¹H NMR (DMSO-d₆, 500 MHz) δ 12.77 (br s, 1H), 7.77-7.72 (m, 1H), 7.66-7.60 (m, 1H), 7.52 (s, 1H).

### Step B: Preparation of methyl 4-oxo-4,5-dihydrothieno[3,2-c]pyridine-7-carboxylate

To a nitrogen purged solution of 7-bromothieno[3,2-c]pyridin-4(5H)-one (i.e. the product of Step A) (10.0 g, 43.5 mmol) in methanol (200 mL) was added triethylamine (13.2 g, 130.4 mmol) followed by dichloro-[1,1′-bis(diphenylphosphino)ferrocene]-palladium(II) (3.1 g, 4.34 mmol). The reaction mixture was sealed in a steel bomb, subjected to 150 psig of carbon monoxide and heated to 100° C. for 16 h. The reaction mixture was cooled to room temperature, filtered through Celite® diatomaceous earth filter aid, rinsing with 50% solution of methanol in dichloromethane (2 L) and concentrated under reduced pressure. The resultant material was triturated with a 50% solution of diethyl ether in n-pentane (200 mL) and filtered to give the title compound as a solid (4.0 g).

¹H NMR (DMSO-d₆, 500 MHz) δ 12.05 (br s, 1H), 8.03 (s, 1H), 7.73-7.71 (m, 1H), 7.54-7.50 (m, 1H), 3.86 (s, 3H).

### Step C: Preparation of methyl 4-bromo-thieno[3,2-c]pyridine-7-carboxylate

To a stirred solution of methyl 4-oxo-4,5-dihydrothieno[3,2-c]pyridine-7-carboxylate (i.e. the product of Step B) (5.0 g, 23.9 mmol) in 1,2-dichloroethane (50 mL) was added phosphorous oxybromide (13.7 g, 47.8 mmol) portionwise and heated at 90° C. for 16 h. The reaction mixture was poured over a saturated aqueous sodium bicarbonate solution (100 mL) and extracted with dichloromethane (2x). The combined extracts were washed with a brine solution, dried over anhydrous sodium sulfate, filtered, and concentrated under reduced pressure and purified by silica gel column chromatography (eluting with 30% ethyl acetate in petroleum ether) to yield the title compound a solid (3.7 g).

¹H NMR (CDCl₃, 500 MHz) δ 8.87 (s, 1H), 7.72 (d, 1H), 7.57 (d, 1H), 4.06 (s, 3H).

### Step D: Preparation of methyl 4-acetyl-thieno[3,2-c]pyridine-7-carboxylate

To a solution of methyl 4-bromo-thieno[3,2-c]pyridine-7-carboxylate (i.e. the product of Step C) (4.0 g, 14.7 mmol) in toluene (40 mL) was added tributyl(1-ethoxyvinyl)stannane (7.0 g, 19.1 mmol) and purged with nitrogen for 15 min. Bis(triphenylphosphine)palladium(II) chloride (1.0 g, 1.47 mmol) was added to the reaction mixture, stirred at 100° C. for 16 h and cooled to room temperature. The reaction mixture was diluted with hydrochloric acid (1.5 N aqueous solution, 50 mL), stirred for 10 h, basified with a saturated aqueous sodium bicarbonate solution (80 mL) and extracted with ethyl acetate (100 mL). The organic extract was washed with a saturated brine solution, dried over anhydrous sodium sulfate, filtered, concentrated under reduced pressure and purified by silica gel column chromatography (eluting with a 50% ethyl acetate in petroleum ether) to yield the title compound as a solid (2.1 g).

¹H NMR (CDCl₃, 500 MHz) δ 9.18 (s, 1H), 8.41 (d, 1H), 7.80 (d, 1H), 4.10 (s, 3H), 2.87 (s, 3H).

### Step E: Preparation of methyl 4-[(2Z)-3-(3,5-dichlorophenyl)-4,4,4-trifluoro-1-oxo-2-buten-1-yl]-thieno[3,2-c]pyridine-7-carboxylate

To a stirred solution of methyl 4-acetyl-thieno[3,2-c]pyridine-7-carboxylate (i.e. the product of Step D) (3.5 g, 12.8 mmol) in dichloromethane (40 mL) was added 1-(3,5-dichlorophenyl)-2,2,2-trifluoroethan-1-one (3.4 g, 14.1 mmol), potassium carbonate (2.0 g, 14.9 mmol) and triethylamine (0.15 g, 1.49 mmol). The reaction mixture heated at the boiling point of the reaction mixture for 10 h, cooled to room temperature and concentrated under reduced pressure. LCMS of the resultant material indicated the presence of an aldol condensation product. To a stirred solution of the resultant material in acetonitrile (80 mL) was added hexachloroethane (6.0 g, 25.2 mmol), triethylamine (7.6 g, 75.5 mmol) and triphenylphosphine (6.6 g, 25.2 mmol). The reaction mixture was heated at 90° C. for 24 h, cooled to room temperature, poured over ice water (100 mL) and extracted with ethyl acetate (2×). The organic extract was washed with a saturated brine solution, dried over anhydrous sodium sulfate, filtered, concentrated under reduced pressure and purified by silica gel column chromatography (eluting with a 10% ethyl acetate in petroleum ether) to yield the title compound as a solid (2.1 g).

¹H NMR (CDCl₃, 500 MHz) δ 9.20 (s, 1H), 8.25-8.21 (m, 2H), 7.82 (d, 1H), 7.37 (s, 1H), 7.21-7.18 (m, 2H), 4.11 (s, 3H).

### Step F: Preparation of methyl 4-[5-(3,5-dichlorophenyl)-4,5-dihydro-5-(tri-fluoromethyl)-3-isoxazolyl]-thieno[3,2-c]pyridine-7-carboxylate

To a stirred solution of methyl 4-[(2Z)-3-(3,5-dichlorophenyl)-4,4,4-trifluoro-1-oxo-2-buten-1-yl]-thieno[3,2-c]pyridine-7-carboxylate (i.e. the product of Step E) (2.1 g, 4.58 mmol) in dichloromethane (30 mL) at 0° C. was added hydroxylamine (50% aqueous solution) (0.34 g, 11.4 mmol) followed by 1,8-diazabicyclo[5.4.0]undec-7-ene (1.39 g, 9.15 mmol). The reaction mixture was allowed to warm to room temperature over a period of 5 h. To the reaction mixture was added ice water (50 mL) and extracted with dichloromethane (2x). The combined organic extracts were dried over anhydrous sodium sulfate, filtered, concentrated under reduced pressure, triturated with a 10% solution of diethyl ether in n-pentane (40 mL), filtered and dried to yield the title compound (1.8 g).

¹H NMR (CDCl₃, 500 MHz) δ 9.10 (s, 1H), 8.33 (d, 1H), 7.98 (d, 1H), 7.56 (s, 2H), 7.44 (s, 1H), 4.48-4.42 (m, 1H), 4.13-4.06 (m, 4H).

### Step G: Preparation of 4-[5-(3,5-dichlorophenyl)-4,5-dihydro-5-(trifluoromethyl)-3-isoxazolyl]-thieno[3,2-c]pyridine-7-carboxylic acid

To a stirred solution of methyl 4-[5-(3,5-dichlorophenyl)-4,5-dihydro-5-(tri-fluoromethyl)-3-isoxazolyl]-thieno[3,2-c]pyridine-7-carboxylate (i.e. the product of Step F) (1.8 g, 3.80 mmol) in tetrahydrofuran (15 mL) and water (5 mL) at 0° C. was added lithium hydroxide monohydrate (0.073 g, 1.93 mmol), allowed to warm to room temperature over 5 h and concentrated under reduced pressure. The resultant material was acidified with hydrochloric acid (1.0 N aqueous solution) to a pH of 6. The resultant solid was filtered, washed with water (40 mL) and dried diluted to yield the title compound as a solid (1.6 g).

¹H NMR (DMSO-d₆, 500 MHz) δ 9.05 (s, 1H), 8.21 (d, 1H), 8.15 (d, 1H), 7.82-7.80 (m, 1H), 7.85-7.80 (m, 2H), 4.56 (d, 1H), 4.38 (d, 1H).

### Step H: Preparation of 4-[5-(3,5-dichlorophenyl)-4,5-dihydro-5-(trifluoro-methyl)-3-isoxazolyl]-N-3-thietanylthieno[3,2-c]pyridine-7-carboxamide. (Compound 27)

To a solution of 4-[5-(3,5-dichlorophenyl)-4,5-dihydro-5-(trifluoromethyl)-3-isoxazolyl]-thieno[3,2-c]pyridine-7-carboxylic acid (i.e. the product of Step G) (0.9 g, 2.0 mmol) in N,N-dimethylformamide (8 mL) were added O-(7-azabenzotriazol-1-yl)-N,N,N′,N′-tetramethyluronium hexafluorophosphate (HATU) (1.1 g, 2.9 mmol), diisopropyl-ethylamine (758 mg, 5.87 mmol) and 3-aminothietane (295 mg, 2.35 mmol). The reaction mixture was stirred at ambient temperature for 16 h and poured over ice cold water (80 mL). The resultant solid was filtered, washed with water (50 mL), and dried to yield the title compound, a compound of the present invention, as a solid (600 mg).

¹H NMR (DMSO-d₆, 500 MHz) δ 9.58 (d, 1H), 9.13 (s, 1H), 8.18 (d, 1H), 8.12 (d, 1H), 7.83-7.78 (m, 3H), 5.37-5.28 (m, 1H), 4.57 (d, 1H), 4.37 (d, 1H), 3.65-3.60 (m, 2H), 3.27-3.22 (m, 2H).

### Example 5

### Preparation of 7-[5-(3,5-dichlorophenyl)-4,5-dihydro-5-(trifluoromethyl)-3-isoxazolyl]-N-[(1R)-1-methyl-2-(methylamino)-2-oxo-ethyl]thieno[2,3-c]pyridine-4-carboxamide. (Compound 7)

### Step A: Preparation of 7-methoxythieno[2,3-c]pyridine

To a solution of 7-chloro-thieno[2,3-c]pyridine (10.0 g, 59.2 mmol) in N,N-dimethylformamide (100 mL) was added sodium methoxide (30% solution in methanol) (21 mL, 118.3 mmol). The reaction mixture was heated in a sealed tube at 140° C. for 1 h and cooled to room temperature. The resultant solution of the title compound in N,N-dimethylformamide was used directly in Step B.

¹H NMR (CDCl₃, 500 MHz) δ 8.02-8.00 (m, 1H), 7.70-7.65 (m, 1H), 7.38-7.24 (m, 2H), 4.18 (s, 3H).

### Step B: Preparation of thieno[2,3-c]pyridin-7(6H)-one

To a N,N-dimethylformamide solution of 7-methoxythieno[2,3-c]pyridine (i.e. the product of Step A) (9.7 g, 58.8 mmol) was added additional N,N-dimethylformamide (30 mL), and pyridine hydrochloride (34.0 g, 293.9 mmol). The reaction mixture was heated in a sealed tube at 100° C. for 10 h, poured into a saturated aqueous sodium bicarbonate solution (500 mL) and extracted with ethyl acetate (2×). The combined organic extracts were washed with a brine solution, dried over anhydrous sodium sulfate, filtered and concentrated under reduced pressure. The resultant material was triturated with a 30% solution of diethyl ether in pentane (50 mL) to give the title compound as a solid (5.0 g).

¹H NMR (DMSO-d₆, 500 MHz) δ 11.49 (br s, 1H), 8.04-8.03 (m, 1H), 7.39-7.38 (m, 1H), 7.28-7.26 (m, 1H), 6.73-6.71 (m, 1H).

### Step C: Preparation of 4-bromothieno[2,3-c]pyridin-7(6H)-one

To a solution of 4-bromothieno[2,3-c]pyridin-7(6H)-one (i.e. the product of Step B) (5.0 g, 32.9 mmol) in N,N-dimethylformamide (200 mL) at 0° C. was added N-bromo-succinimide (7.0 g, 39.5 mmol) portionwise and stirred at room temperature for 16 h. The reaction mixture was poured over ice water (250 mL), filtered and washed with water (50 mL). The solid collected was dried under reduced pressure to provide the title compound as a solid (5.5 g).

¹H NMR (DMSO-d₆, 500 MHz) δ 11.8 (br s, 1H), 8.17-8.16 (m, 1H), 7.65-7.64 (m, 1H), 7.35-7.34 (m, 1H).

### Step D: Preparation of methyl 7-oxo-6,7-dihydrothieno[2,3-c]pyridine-4-carboxylate

To a nitrogen purged solution of 4-bromothieno[2,3-c]pyridin-7(6H)-one (i.e. the product of Step C) (5.5 g, 23.9 mmol) in methanol (200 mL) was added triethylamine (7.25 g, 71.7 mmol) followed by dichloro-[1,1′-bis(diphenylphosphino)ferrocene]-palladium(II) (1.74 g, 2.39 mmol). The reaction mixture was sealed in a steel bomb, subjected to 150 psig of carbon monoxide and heated to 100° C. for 16 h. The reaction mixture was cooled to room temperature, filtered through Celite® diatomaceous earth filter aid, rinsing with ethyl acetate (100 mL). The filtrate was washed with water followed by a brine solution, dried over anhydrous sodium sulfate and concentrated under reduced pressure. The resultant material was triturated with a 50% solution of diethyl ether in n-pentane (100 mL) and filtered to give the title compound as a solid (4.0 g).

¹H NMR (DMSO-d₆, 500 MHz) δ 8.21-8.19 (m, 1H), 8.05-8.02 (m, 1H), 7.94-7.98 (m, 1H), 3.82 (s, 3H).

### Step E: Preparation of methyl 7-bromothieno[2,3-c]pyridine-4-carboxylate

To a stirred solution of methyl 7-oxo-6,7-dihydrothieno[2,3-c]pyridine-4-carboxylate (i.e. the product of Step E) (4.0 g, 19.1 mmol) in 1,2-dichloroethane (40 mL) was added phosphorous oxybromide (10.9 g, 38.3 mmol) portionwise and heated at 90° C. for 16 h. The reaction mixture was poured over a saturated aqueous sodium bicarbonate solution (150 mL) and extracted with dichloromethane (2x). The combined extracts were washed with a brine solution, dried over anhydrous sodium sulfate, filtered, and concentrated under reduced pressure and purified by silica gel column chromatography (eluting with 80% ethyl acetate in petroleum ether) to yield the title compound a solid (7.0 g).

¹H NMR (CDCl₃, 500 MHz) δ 8.96 (s, 1H), 8.22 (d, 1H), 7.93 (d, 1H), 4.03 (s, 3H).

### Step F: Preparation of methyl 7-acetylthieno[2,3-c]pyridine-4-carboxylate

To a solution of methyl 7-bromothieno[2,3-c]pyridine-4-carboxylate (i.e. the product of Step E) (5.0 g, 18.4 mmol) in toluene (50 mL) was added tributyl(1-ethoxyvinyl)stannane (8.6 g, 23.9 mmol) and purged with nitrogen for 15 min. Bis(triphenylphosphine)-palladium(II) chloride (1.28 g, 1.84 mmol) was added to the reaction mixture, stirred at 100° C. for 16 h and cooled to room temperature. The reaction mixture was diluted with hydrochloric acid (1.0 N aqueous solution, 60 mL), stirred for 10 h, basified with a saturated aqueous sodium bicarbonate solution (80 mL) and extracted with ethyl acetate (100 mL). The organic extract was washed with a saturated brine solution, dried over anhydrous sodium sulfate, filtered, concentrated under reduced pressure and purified by silica gel column chromatography (eluting with 70% ethyl acetate in petroleum ether) to yield the title compound as a solid (3.0 g).

¹H NMR (CDCl₃, 500 MHz) δ 9.26 (s, 1H), 8.24 (d, 1H), 8.03 (d, 1H), 4.08 (s, 3H), 2.91 (s, 3H).

### Step G: Preparation of methyl 7-[(2Z)-3-(3,5-dichlorophenyl)-4,4,4-trifluoro-1-oxo-2-buten-1-yl]thieno[2,3-c]pyridine-4-carboxylate

To a stirred solution of methyl 7-acetylthieno[2,3-c]pyridine-4-carboxylate (i.e. the product of Step F) (3.0 g, 12.8 mmol) in 2-methyltetrahydrofuran (30 mL) was added molecular sieves and potassium carbonate (8.82 g, 63.8 mmol). The reaction mixture was heated to 80° C. and 1-(3,5-dichlorophenyl)-2,2,2-trifluoroethan-1-one (6.2 g, 25.6 mmol) was added portion-wise. The reaction mixture was heated at 90° C. for 10 h, cooled to room temperature and filtered through Celite® diatomaceous earth filter aid rinsing with ethyl acetate (100 mL). The filtrate was concentrated under reduced pressure and purified by silica gel column chromatography (eluting with 80% ethyl acetate in petroleum ether) to yield the title compound as a solid (2.0 g).

¹H NMR (CDCl₃, 500 MHz) δ 9.30 (s, 1H), 8.41-8.39 (m, 1H), 8.25 (d, 1H), 8.02 (d, 1H), 7.42-7.39 (m, 1H), 7.23-7.20 (m, 2H), 4.08 (s, 3H).

### Step H: Preparation of methyl 7-[5-(3,5-dichlorophenyl)-4,5-dihydro-5-(tri-fluoromethyl)-3-isoxazolyl]thieno[2,3-c]pyridine-4-carboxylate

To a stirred solution of methyl 7-[(2Z)-3-(3,5-dichlorophenyl)-4,4,4-trifluoro-1-oxo-2-buten-1-yl]thieno[2,3-c]pyridine-4-carboxylate (i.e. the product of Step G) (2.0 g, 4.36 mmol) in dichloromethane (30 mL) at 0° C. was added hydroxylamine (50% aqueous solution) (0.7 mL, 10.9 mmol) followed by 1,8-diazabicyclo[5.4.0]undec-7-ene (1.32 g, 8.71 mmol). The reaction mixture was allowed to warm to room temperature over a period of 5 h. The reaction mixture was poured over ice water (50 mL) and extracted with dichloromethane (2x). The combined organic extracts were dried over anhydrous sodium sulfate, filtered, concentrated under reduced pressure, triturated with a 10% solution of diethyl ether in n-pentane (40 mL), filtered and dried to yield the title compound (1.5 g).

¹H NMR (CDCl₃, 500 MHz) δ 9.17 (s, 1H), 8.28-8.27 (m, 1H), 7.99-7.97 (m, 1H), 7.58-7.55 (m, 2H), 7.44-7.40 (m, 1H), 4.48-4.45 (m, 1H), 4.10-4.06 (m, 1H), 4.05 (s, 3H).

### Step I: Preparation of 7-[5-(3,5-dichlorophenyl)-4,5-dihydro-5-(trifluoromethyl)-3-isoxazolyl]thieno[2,3-c]pyridine-4-carboxylic acid

To a stirred solution of methyl 7-[5-(3,5-dichlorophenyl)-4,5-dihydro-5-(trifluoromethyl)-3-isoxazolyl]thieno[2,3-c]pyridine-4-carboxylate (i.e. the product of Step H) (1.5 g, 3.16 mmol) in tetrahydrofuran (16 mL) and water (4 mL) at 0° C. was added lithium hydroxide monohydrate (0.4 g, 9.5 mmol), allowed to warm to room temperature over 6 h and concentrated under reduced pressure. The resultant material was acidified with hydrochloric acid (1.0 N aqueous solution) to a pH of 6. The resultant solid was filtered, washed with water (40 mL) and dried diluted to yield the title compound as a solid (1.2 g).

¹H NMR (DMSO-d₆, 500 MHz) δ 9.10 (s, 1H), 8.40-8.37 (m, 1H), 8.28-8.25 (m, 1H), 7.82-7.78 (m, 3H), 4.58-4.52 (m, 1H), 4.45-4.40 (m, 1H).

### Step J: Preparation of 7-[5-(3,5-dichlorophenyl)-4,5-dihydro-5-(trifluoro-methyl)-3-isoxazolyl]-N-[(1R)-1-methyl-2-(methylamino)-2-oxoethyl]-thieno[2,3-c]pyridine-4-carboxamide. (Compound 7)

To a solution of 7-[5-(3,5-dichlorophenyl)-4,5-dihydro-5-(trifluoromethyl)-3-isoxazolyl]thieno[2,3-c]pyridine-4-carboxylic acid (i.e. the product of Step I) 0-(7-aza-benzotriazol-1-yl)-N,N,N′,N′-tetramethyluronium hexafluorophosphate (HATU) (0.25 g, 0.65 mmol), diisopropylethylamine (0.2 mL, 1.30 mmol) and (2R)-2-amino-N-methyl-propanamide (64.7 mg, 0.52 mmol). The reaction mixture was stirred at ambient temperature for 16 h and poured over ice cold water (20 mL). The resultant solid was filtered, washed with water (10 mL), and dried to yield the title compound, a compound of the present invention, as a solid (140 mg).

¹H NMR (DMSO-d₆, 500 MHz) δ 8.91-8.89 (m, 1H), 8.32-8.30 (m, 1H), 8.00-7.98 (m, 1H), 7.82-7.78 (m, 3H), 7.53-7.48 (m, 1H), 7.07-7.03 (m, 1H), 4.56-4.37 (m, 3H), 1.38-1.36 (m, 3H).

### Example 6

### Preparation of N-cyclopropyl-4-[4,5-dihydro-5-(trifluoromethyl)-5-[3(trifluoro-methyl)phenyl]-3-isoxazolyl]furo[2,3-c]pyridine-7-carboxamide. (Compound 15)

### Step A: Preparation of 3-furanacrylic acid

A stirred mixture of 3-furancarboxaldehyde (20 g, 208 mmol), piperidine (16 mL) and malonic acid (54.3 g, 522 mmol) in pyridine (400 mL) was heated to 65° C. for 16 h, acidified with hydrochloric acid (6.0 N aqueous solution) to a pH of 2 and extracted with ethyl acetate (2×). The combined organic extracts were washed with a brine solution (250 mL), dried over anhydrous sodium sulfate, filtered and concentrated under reduced pressure to provide the title compound as a solid (20 g).

¹H NMR (CDCl₃, 400 MHz) δ 7.70-7.68 (d, 1H), 7.66 (s, 1H), 7.45 (s, 1H), 6.62 (s, 1H), 6.18-6.14 (d, 1H).

LCMS: m/z: 139 [M+H]⁺

### Step B: Preparation of 3-(3-furanyl)-2-propenoyl azide

To a solution of 3-furanacrylic acid (i.e. the product of Step A) (30 g, 217 mmol) in tetrahydrofuran (150 mL) was added triethylamine (36.3 mL, 258 mmol) and diphenylphosphoral azide (DPPA) (50.9 mL, 237 mmol), stirred for 4 h. The reaction mixture was poured over an ice cold saturated aqueous sodium bicarbonate solution (500 mL) and extracted with ethyl acetate (2×). The combined organic extracts were washed with a brine solution (250 mL), dried over anhydrous sodium sulfate, filtered and concentrated under reduced pressure to give the title compound as a solid (40 g).

¹H NMR (CDCl₃, 400 MHz) δ 7.70-7.63 (t, 2H), 7.45 (s, 1H), 6.59 (s, 1H), 6.16-6.12 (d, 1H).

### Step C: Preparation of furo[2,3-c]pyridin-7(6H)-one

To a stirred solution of 3-(3-furanyl)-2-propenoyl azide (i.e. the product of Step B) (35 g, 215 mmol) in 1,2-dichlorobenzene (250 mL) was added iodine (0.21 g, 0.85 mmol) and heated at 180° C. for 2 h. The reaction mixture was concentrated under reduced pressure and purified by silica gel column chromatography (eluting with 50% ethyl acetate in petroleum ether) to yield the title compound as a solid (20.0 g).

¹H NMR (CDCl₃, 400 MHz) δ 7.79 (d, 1H), 7.28-7.26 (d, 1H), 6.73-6.72 (d, 1H), 6.61-6.59 (d, 1H).

LCMS m/z 136 [M+H]⁺

### Step D: Preparation of 4-bromofuro[2,3-c]pyridin-7(6H)-one

To a solution of furo[2,3-c]pyridin-7(6H)-one (i.e. the product of Step C) (14 g, 104 mmol) in acetic acid (140 mL) at 0° C. was added bromine (5.3 mL, 104 mmol) and stirred 2 h. The reaction mixture was concentrated under reduced pressure, basified with a saturated aqueous sodium bicarbonate solution to a pH of 8, stirred for 30 min and filtered. The resultant solid was washed with water (250 mL) and dried to give the title compound as a solid (14 g).

¹H NMR (CDCl₃, 500 MHz) δ 12.43 (br s, 1H), 7.84 (d, 1H), 7.38 (s, 1H), 6.78 (d, 1H).

LCMS: m/z: 214 [M+H]⁺

### Step E: Preparation of 4-bromo-7-chlorofuro[2,3-c]pyridine

A solution of 4-bromofuro[2,3-c]pyridin-7(6H)-one (i.e. the product of Step D) (14 g, 65.7 mmol) in phosphorous oxychloride (250 mL) was heated at 120° C. for 12 h. The reaction mixture was concentrated under reduced pressure, basified with a saturated aqueous potassium carbonate solution to a pH of 10 and extracted with dichloromethane (2x). The combined organic extracts were washed with a brine solution (250 mL), dried over anhydrous sodium sulfate, decanted, concentrated under reduced pressure and purified by silica gel column chromatography (eluting with 10% ethyl acetate in petroleum ether) to yield the title compound as a solid (8.0 g).

¹H NMR (CDCl₃, 400 MHz) δ 8.30 (s, 1H), 7.88-7.87 (d, 1H), 6.93-6.92 (d, 1H).

LCMS: m/z: 232 [M+H]⁺

### Step F: Preparation of 1-(7-chlorofuro[2,3-c]pyridin-4-yl)ethan-1-one

To a stirred solution of 4-bromo-7-chlorofuro[2,3-c]pyridine (i.e. the product of Step E) (8 g, 34.6 mmol) in toluene (200 mL) was added tributyl(1-ethoxyvinyl)stannane (15.3 mL, 45.0 mmol) and bis(triphenylphosphine)palladium(II) chloride (2.43 g, 3.46 mmol) and heated at 100° C. for 3 h. The reaction mixture was cooled to 0° C., hydrochloric acid (2 N aqueous solution) (270 mL) was added, stirred at room temperature for 1 h, poured over an ice cold saturated aqueous sodium bicarbonate solution (500 mL) and extracted with ethyl acetate (2×). The combined organic extracts were washed with a brine solution (250 mL), dried over anhydrous sodium sulfate, decanted, concentrated under reduced pressure and purified by silica gel column chromatography (eluting with 10% ethyl acetate in petroleum ether) to yield the title compound as a solid (3.4 g).

¹H NMR (CDCl₃, 400 MHz) δ 8.76-8.75 (d, 1H), 7.94-7.93 (d, 1H), 7.60-7.59 (d, 1H), 2.72 (s, 3H).

LCMS m/z: 196 [M+H]⁺

### Step G: Preparation of methyl 4-acetylfuro[2,3-c]pyridine-7-carboxylate

To a stirred solution of 1-(7-chlorofuro[2,3-c]pyridin-4-yl)ethan-1-one (i.e. the product of Step F) (3 g, 15.3 mmol) in methanol (160 mL), was added dichloro-[1,1′-bis(diphenylphosphino)ferrocene]palladium(II) (1.12 g, 1.53 mmol) and triethylamine (6.48 mL, 46.1 mmol). The reaction mixture was sealed in a steel bomb, subjected to 100 psig of carbon monoxide and heated to 100° C. for 16 h. The reaction mixture was cooled to room temperature, filtered through Celite® diatomaceous earth filter aid, concentrated under reduced pressure and purified by silica gel column chromatography (eluting with 30% ethyl acetate in petroleum ether) to yield the title compound as a solid (1.5 g).

¹H NMR (CDCl₃, 400 MHz) δ 9.06 (s, 1H), 8.03 (d, 1H), 7.64-7.63 (d, 1H), 4.13 (s, 3H), 2.78 (s, 3H).

LCMS: m/z: 220 [M+H]⁺

### Step H: Preparation of methyl 4-[(2Z)-3-[3(trifluoromethyl)phenyl]-4,4,4-trifluoro-1-oxo-2-buten-1-yl]furo[2,3-c]pyridine-7-carboxylate

To a stirred solution of methyl 4-acetylfuro[2,3-c]pyridine-7-carboxylate (i.e. the product of Step G) (1.0 g, 4.56 mmol) in toluene (30 mL) was added 2,2,2-trifluoro-1-[3-(trifluoromethyl)phenyl]ethan-1-one (2.2 g, 9.13 mmol) and triethylamine (1.92 mL, 13.7 mmol). The reaction mixture was heated at 100° C. for 16 h. Following consumption of methyl 4-acetylfuro[2,3-c]pyridine-7-carboxylate, acetic anhydride (0.46 g, 4.56 mmol) was added to the reaction mixture, stirred at 100° C. for 12 h, cooled, basified with sodium hydroxide (1 N aqueous solution) to a pH of 12 and extracted with ethyl acetate (2×). The combined organic extracts were washed with a brine solution (250 mL), dried over anhydrous sodium sulfate, decanted, concentrated under reduced pressure and purified by silica gel column chromatography (eluting with 10% ethyl acetate in petroleum ether) to yield the title compound as a solid (0.6 g).

¹H NMR (CDCl₃, 400 MHz) δ 8.87 (s, 1H), 8.01 (s, 1H), 7.54-7.49 (m, 3H), 7.43-7.38 (m, 3H), 4.10 (s, 3H).

LCMS: m/z: 444 [M+H]⁺

### Step I Preparation of methyl 4-[4,5-dihydro-5-(trifluoromethyl)-5-[3(tri-fluoromethyl)phenyl]-3-isoxazolyl]furo[2,3-c]pyridine-7-carboxylate

To a solution of methyl 4-[(2Z)-3-[3(trifluoromethyl)phenyl]-4,4,4-trifluoro-1-oxo-2-buten-1-yl]furo[2,3-c]pyridine-7-carboxylate (i.e. the product of Step H) (0.6 g, 1.35 mmol) in dichloromethane (20 mL) was added hydroxylamine (50% aqueous solution) (0.22 mL, 3.38 mmol) followed by 1,8-diazabicyclo[5.4.0]undec-7-ene (0.51 mL, 3.38 mmol) and stirred for 6 h. The reaction mixture was poured into ice water (200 mL) and extracted with DCM (2x). The combined organic extracts were washed with a brine solution (100 mL), dried over anhydrous sodium sulfate, decanted, concentrated under reduced pressure and purified by silica gel column chromatography (eluting with 10% ethyl acetate in petroleum ether) to yield the title compound as a solid (0.33 g).

¹H NMR (CDCl₃, 400 MHz) δ 8.52 (s, 1H), 8.03 (s, 1H), 7.93 (s, 1H), 7.86-7.84 (d, 1H), 7.74-7.72 (d, 1H), 7.65-7.61 (t, 1H), 7.53-7.52 (d, 1H), 4.33-4.32 (d, 1H), 4.12 (s, 3H), 3.94-3.90 (d, 1H).

LCMS: m/z: 459 [M+H]⁺

### Step J: Preparation of 4-[4,5-dihydro-5-(trifluoromethyl)-5-[3(trifluoro-methyl)phenyl]-3-isoxazolyl]furo[2,3-c]pyridine-7-carboxylic acid

To a solution of methyl 4-[4,5-dihydro-5-(trifluoromethyl)-5-[3(tri-fluoromethyl)phenyl]-3-isoxazolyl]furo[2,3-c]pyridine-7-carboxylate (i.e. the product of Step I) (0.33 g, 0.72 mmol) in tetrahydrofuran (40 mL) and water (1 mL) was added lithium hydroxide monohydrate (0.09 g, 2.16 mmol), stirred for 3 hours and concentrated under reduced pressure. The resultant material was acidified with hydrochloric acid (1.0 N aqueous solution) to a pH of 2 and filtered. The resultant solid was washed with water (50 mL) and dried to yield the title compound as a solid (0.23 g).

¹H NMR (CDCl₃, 400 MHz) δ 8.52 (s, 1H), 8.03 (s, 1H), 7.93 (s, 1H), 7.86-7.84 (d, 1H), 7.74-7.72 (d, 1H), 7.65-7.61 (t, 1H), 7.53-7.52 (d, 1H), 4.33-4.32 (d, 1H), 3.94-3.90 (d, 1H).

LCMS: m/z: 445 [M+H]⁺

### Step K: Preparation of N-cyclopropyl-4-[4,5-dihydro-5-(trifluoromethyl)-5-[3(trifluoromethyl)phenyl]-3-isoxazolyl]furo[2,3-c]pyridine-7-carboxamide. (Compound 15)

To a stirred solution of 4-[4,5-dihydro-5-(trifluoromethyl)-5-[3(trifluoro-methyl)phenyl]-3-isoxazolyl]furo[2,3-c]pyridine-7-carboxylic acid (i.e. the product of Step J) (0.10 g, 0.22 mmol) in dichloromethane (8 mL) and N,N-dimethylformamide (0.1 mL) was added cyclopropylamine (0.012 mL, 0.22 mmol) and triethylamine (0.09 mL, 0.67 mmol) followed by propylphosphonic anhydride (0.28 mL, 0.45 mmol). The reaction mixture was stirred for 16 h, poured into ice water (100 mL) and extracted with dichloromethane (2x). The combined organic extracts were washed with a brine solution (50 mL), dried over anhydrous sodium sulfate, decanted, concentrated under reduced pressure and purified by silica gel column chromatography (eluting with 20% ethyl acetate in petroleum ether) to yield the title compound, a compound of the present invention, as a solid (0.05 g).

¹H NMR (CDCl₃, 400 MHz) δ 8.32 (s, 1H), 8.05-8.04 (d, 1H), 8.00 (s, 1H), 7.91 (br s, 1H), 7.86-7.84 (d, 1H), 7.73-7.71 (d, 1H), 7.64-7.60 (t, 1H), 7.47-7.46 (d, 1H), 4.31-4.27 (d, 1H), 3.92-3.88 (d, 1H), 3.02-2.98 (m, 1H), 0.94-0.89 (m, 2H), 0.73-0.69 (m, 2H).

LCMS: m/z: 484 [M+H]⁺

### Example 7

### Preparation of N-cyclopropyl-4-[4,5-dihydro-5-(trifluoromethyl)-5-[3(trifluoro-methyl)phenyl]-3-isoxazolyl]furo[2,3-c]pyridine-7-carboxamide. (Compound 9)

To a stirred solution of 4-[4,5-dihydro-5-(trifluoromethyl)-5-[3(trifluoro-methyl)phenyl]-3-isoxazolyl]furo[2,3-c]pyridine-7-carboxylic acid (i.e. the product of Example 6 Step J) (0.1 g, 0.22 mmol), ammonium chloride (0.016 g, 0.29 mmol), 0-(7-aza-benzotriazol-1-yl)-N,N,N′,N′-tetramethyluronium hexafluorophosphate (HATU) (0.17 g, 0.45 mmol) in N,N-dimethylformamide (5 mL) was added triethylamine (0.09 mL, 0.67 mmol) and stirred for 8 h. The reaction mixture was poured over ice water (100 mL) and extracted with ethyl acetate (2×). The combined organic extracts were washed with a brine solution (50 mL), dried over anhydrous sodium sulfate, decanted, concentrated under reduced pressure and purified by silica gel column chromatography (eluting with 10% ethyl acetate in petroleum ether) to yield the title compound, a compound of the present invention, as a solid (0.065 g).

¹H NMR (DMSO-d₆, 500 MHz) δ 8.66 (s, 1H), 8.50-8.49 (d, 1H), 8.23 (br s, 1H), 7.99-7.98 (d, 1H), 7.93-7.91 (d, 2H), 7.83-7.80 (t, 2H), 7.41-7.40 (d, 1H), 4.73-4.69 (d, 1H), 4.53-4.50 (d, 1H).

LCMS: m/z: 444 [M+H]⁺

### Example 8

### Preparation of N-[[4-[4,5-dihydro-5-(trifluoromethyl)-5-[3-(trifluoromethyl)phenyl]-3-isoxazolyl]furo[2,3-c]pyridin-7-yl]methyl]acetamide. (Compound 1)

### Step A: Preparation of 4-[4,5-dihydro-5-(trifluoromethyl)-5-[3(tri-fluoromethyl)phenyl]-3-isoxazolyl]furo[2,3-c]pyridine-7-methanol

To a stirred solution of methyl 4-[4,5-dihydro-5-(trifluoromethyl)-5-[3(tri-fluoromethyl)phenyl]-3-isoxazolyl]furo[2,3-c]pyridine-7-carboxylate (i.e. the product of Example 1 Step I) (0.8 g, 1.74 mmol) in methanol (40 mL), tetrahydrofuran (6 mL) and water (2 mL) at 0° C. was added sodium borohydride (0.46 g, 12.2 mmol). The reaction mixture was stirred at room temperature for 24 h, poured over ice water (100 mL) and extracted with ethyl acetate (2×). The combined organic extracts were washed with a brine solution (100 mL), dried over anhydrous sodium sulfate, decanted, concentrated under reduced pressure and triturated with n-pentane to provide the title compound as a solid (0.5 g).

¹H NMR (CDCl₃, 400 MHz) δ 8.38 (s, 1H), 7.93 (s, 1H), 7.88-7.84 (t, 2H), 7.73-7.71 (d, 1H), 7.64-7.60 (t, 1H), 7.45 (s, 1H), 5.12 (s, 2H), 4.32-4.28 (d, 1H), 3.93-3.89 (d, 2H).

LCMS: m/z: 431 [M+H]⁺

### Step B: Preparation of 7-chloromethyl-4-[4,5-dihydro-5-(trifluoromethyl)-5-[3(trifluoromethyl)phenyl]-3-isoxazolyl]furo[2,3-c]pyridine

To a stirred solution of 4-[4,5-dihydro-5-(trifluoromethyl)-5-[3(trifluoro-methyl)phenyl]-3-isoxazolyl]furo[2,3-c]pyridine-7-methanol (i.e. the product of Step A) (0.1 g, 0.23 mmol) in toluene (10 mL) at 0° C. was added thionyl chloride (0.1 mL, 0.69 mmol), stirred at room temperature for 3 h, poured over ice water (100 mL), basified with an aqueous ammonia solution to a pH of 14 and extracted with ethyl acetate (2×). The combined organic extracts were washed with a brine solution (50 mL), dried over anhydrous sodium sulfate, decanted and concentrated under reduced pressure to give the title compound as a solid (0.1 g).

¹H NMR (CDCl₃, 400 MHz) δ 8.40 (s, 1H), 7.92 (s, 1H), 7.85-7.83 (d, 1H), 7.73-7.71 (d, 1H), 7.64-7.60 (t, 1H), 7.46-7.45 (d, 1H), 7.18-7.15 (m, 1H), 4.99 (s, 2H), 4.30-4.26 (d, 1H), 3.91-3.87 (d, 1H).

LCMS: m/z: 449 [M+H]⁺

### Step C: Preparation of 2-[[4-[4,5-dihydro-5-(trifluoromethyl)-5-[3-(trifluoro-methyl)phenyl]-3-isoxazolyl]furo[2,3-c]pyridin-7-yl]methyl]-1H-isoindole-1,3(2H)-dione

To a stirred solution of 7-chloromethyl-4-[4,5-dihydro-5-(trifluoromethyl)-5-[3(trifluoromethyl)phenyl]-3-isoxazolyl]furo[2,3-c]pyridine (i.e. the product of Step B) (0.1 g, 0.22 mmol) in N,N-dimethylformamide (5 mL) was added potassium phthalimide (0.061 g, 0.33 mmol), heated at 120° C. for 5 h. The reaction mixture was poured over ice water (100 mL) and extracted with ethyl acetate (2×). The combined organic extracts were washed with a brine solution (50 mL), dried over anhydrous sodium sulfate, decanted and concentrated under reduced pressure to yield the title compound as a solid (0.04 g).

¹H NMR (CDCl₃, 400 MHz) δ 8.27 (s, 1H), 7.91-7.89 (m, 3H), 7.85-7.84 (d, 1H), 7.81-7.79 (d, 1H), 7.77-7.75 (m, 2H), 7.71-7.69 (d, 1H), 7.61-7.57 (t, 1H), 7.41-7.41 (d, 1H), 5.37 (s, 2H), 4.21-4.17 (d, 1H), 3.83-3.79 (d, 1H).

LCMS: m/z: 560 [M+H]⁺

### Step D: Preparation of 4-[4,5-dihydro-5-(trifluoromethyl)-5-[3(trifluoro-methyl)phenyl]-3-isoxazolyl]furo[2,3-c]pyridine-7-methanamine

To a stirred solution of 2-[[4-[4,5-dihydro-5-(trifluoromethyl)-5-[3-(trifluoro-methyl)phenyl]-3-isoxazolyl]furo[2,3-c]pyridin-7-yl]methyl]-1H-isoindole-1,3(2H)-dione (i.e. the product of Step C) (0.4 g, 0.71 mmol) in isopropanol (20 mL) and water (1.2 mL) at 0° C. was added sodium borohydride (0.13 g, 3.57 mmol), stirred for 8 h, poured over ice water (100 mL) and extracted with ethyl acetate (2×). The combined organic extracts were washed with a brine solution (50 mL), dried over anhydrous sodium sulfate, decanted and concentrated under reduced pressure to give the title compound as a liquid (0.3 g), which was used directly in Step E.

### Step E: Preparation of N-[[4-[4,5-dihydro-5-(trifluoromethyl)-5-[3-(trifluoromethyl)phenyl]-3-isoxazolyl]furo[2,3-c]pyridin-7-yl]methyl]acetamide. (Compound 1)

To a stirred solution of 4-[4,5-dihydro-5-(trifluoromethyl)-5-[3(trifluoro-methyl)phenyl]-3-isoxazolyl]furo[2,3-c]pyridine-7-methanamine (i.e. the product of Step D) (0.3 g, 0.69 mmol) in dichloromethane (15 mL) at 0° C. was added acetic anhydride (0.08 mL, 0.83 mmol), pyridine (0.12 mL, 1.39 mmol) and a catalytic amount of 4 dimethylaminopyridine. The reaction mixture was stirred at room temperature for 1 h, poured over ice water (100 mL) and extracted with ethyl acetate (2×). The combined organic extracts were washed with a brine solution (50 mL), dried over anhydrous sodium sulfate, decanted and concentrated under reduced pressure. The resultant material was purified by preparative silica gel thin layer chromatography (eluting with ethyl acetate) to yield the title compound, a compound of the present invention, as a solid (0.033 g).

¹H NMR (DMSO-d₆, 400 MHz) δ 8.58 (s, 1H), 8.53-8.50 (t, 1H), 8.40 (d, 1H), 7.99-7.97 (d, 1H), 7.92-7.90 (d, 2H), 7.83-7.79 (t, 1H), 7.72-7.66 (m, 1H), 7.30 (d, 1H), 4.70-4.62 (m, 3H), 4.48-4.43 (d, 1H), 1.87 (s, 3H).

LCMS: m/z: 472 [M+H]⁺

By the procedures described herein together with methods known in the art, the following compounds of Tables 1 to 144 can be prepared. The following abbreviations are used in the Tables which follow: Me means methyl, OMe means methoxy, Et means ethyl, OEt means ethoxy, n-Pr means n-propyl, i-Pr means isopropyl, c-Pr means cyclopropyl, Ph means phenyl and CN means cyano.

TABLE 1 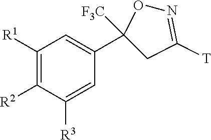
R¹ is Cl, R² is H, R³ is Cl, T is T-1 and X is O.

| J | J |
|---|---|
| —C(═O)NH₂ | —C(═O)NHCH₂(pyrimidin-2-yl) |
| —C(═O)NHMe | —C(═O)NHCH₂(pyridin-2-yl) |
| —C(═O)NHEt | —C(═O)NHCH₂(pyrazin-2-yl) |
| —C(═O)NH-n-Pr | —C(═O)NHCH(—CH₂SCH₂—) |
| —C(═O)NH-i-Pr | —C(═O)NHCH(—CH₂S(═O)CH₂—) |
| —C(═O)NH-c-Pr | —C(═O)NHCH(—CH₂S(═O)₂CH₂—) |
| —C(═O)NHCH₂CH═CH₂ | —C(═O)NHCH(—CH₂N(SO₂Me)CH₂—) |
| —C(═O)NHCH₂C═CH | —C(═O)NHC(CN)(—CH₂CH₂—) |
| —C(═O)NHCH₂CF₃ | —C(═O)NHCH₂C(CN)(—CH₂CH₂—) |
| —C(═O)NHCH═NOMe | —C(═O)NHCH(—CH₂N(SO₂Et)CH₂—) |
| —C(═O)NHCH═NOEt | —C(═O)NHCH(—CH₂N(SO₂CHF₂)CH₂—) |
| —C(═O)NHCH₂-c-Pr | —C(═O)NHCH(—CH₂ONHC(═O)—) |
| —C(═O)NHCH₂CH₂OMe | —C(═O)NHCH(—CH₂ON(Me)C(═O)—) |
| —C(═O)NHCH₂CH₂SMe | —C(═O)NHCH(—CH₂ON(Et)C(═O)—) |
| —C(═O)NHCH₂CH₂S(═O)Me | —C(═O)NHCH(—CH₂ON(CH₂CF₃)C(═O)—) |
| —C(═O)NHCH₂CH₂SO₂Me | —C(═O)NHCH(—CH₂ONHCH₂—) |
| —C(═O)NHCH(Me)CH₂SMe | —C(═O)NHCH(—CH₂ON(SO₂Me)CH₂—) |
| —C(═O)NHCH(Me)CH₂S(═O)Me | —C(═O)NHCH(—CH₂ON(SO₂Et)CH₂—) |
| —C(═O)NHCH(Me)CH₂SO₂Me | —C(═O)NHCH(—CH₂ON(SO₂CHF₂)CH₂—) |
| —C(═O)NHCH₂CO₂Me | —C(═O)NHCH(—CH₂C(═O)NHC(═O)—) |
| —C(═O)NHCH₂C(═O)NH₂ | —C(═O)NHCH(—CH₂C(═O)N(Me)C═O)—) |
| —C(═O)NHCH₂C(═O)NHMe | —C(═O)NHCH(—CH₂C(═O)N(Et)C(═O)—) |
| —C(═O)NHCH₂C(═O)NHOMe | —C(═O)NHCH(—CH₂CH₂C(═O)NHC(═O)—) |
| —C(═O)NHCH₂C(═O)NHEt | —C(═O)NHCH(—CH₂CH₂C(═O)N(Me)C(═O)—) |
| —C(═O)NHCH₂C(═O)NHCH₂CF₃ | —C(═O)NHCH(—CH₂CH₂C(═O)N(Et)C(═O)—) |
| —C(═O)NHCH(Me)C(═O)₂Me | —C(═O)NH(2,4-di-F—Ph) |
| —C(═O)NHCH(Me)C(═O)NH₂ | —C(═O)NH(4-F—Ph) |
| —C(═O)NHCH(Me)C(═O)NHMe | —C(═O)NH(5-F-pyridin-2-yl) |
| —C(═O)NHCH(Me)C(═O)NHOMe | —CH(Me)NHC(═O)Me |
| —C(═O)NHCH(Me)C(═O)NHEt | —CH(Me)NHC(═O)CF₃ |
| —C(═O)NHCH(Me)C(═O)NHCH₂CF₃ | —CH(Me)NHC(═O)Et |
| —CH₂NHC(═O)Me | —CH(Me)NHC(═O)-c-Pr |
| —CH₂NHC(═O)CF₃ | —CH(Me)NHC(═O)CH₂CF₃ |
| —CH₂NHC(═O)Et | —CH₂NHC(═O)CH₂CF₃ |
| —CH₂NHC(═O)-c-Pr | 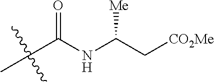 [SMILES: *C(=O)N[C@H](C)CC(=O)OC] |
| 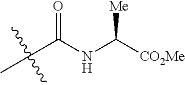 [SMILES: *C(=O)N[C@@H](C)C(=O)OC] | |
| 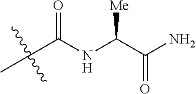 [SMILES: *C(=O)N[C@@H](C)C(N)=O] | 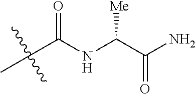 [SMILES: *C(=O)N[C@H](C)C(N)=O] |
| 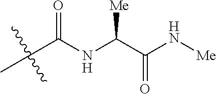 [SMILES: *C(=O)N[C@@H](C)C(=O)NC] | 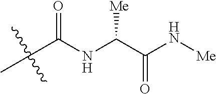 [SMILES: *C(=O)N[C@H](C)C(=O)NC] |
| 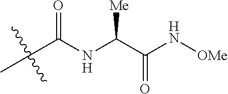 [SMILES: *C(=O)N[C@@H](C)C(=O)NOC] | 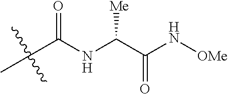 [SMILES: *C(=O)N[C@H](C)C(=O)NOC] |
| 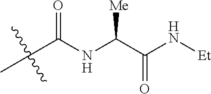 [SMILES: *C(=O)N[C@@H](C)C(=O)NCC] | 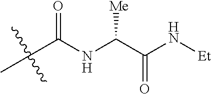 [SMILES: *C(=O)N[C@H](C)C(=O)NCC] |
| 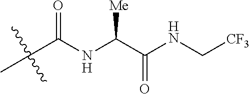 [SMILES: *C(=O)N[C@@H](C)C(=O)NCC(F)(F)F] | 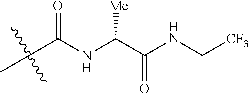 [SMILES: *C(=O)N[C@H](C)C(=O)NCC(F)(F)F] |
| 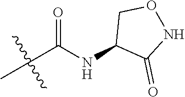 [SMILES: *C(=O)N[C@H]1CONC1=O] | 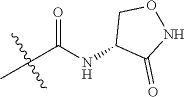 [SMILES: *C(=O)N[C@@H]1CONC1=O] |
| 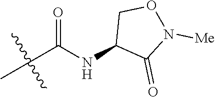 [SMILES: *C(=O)N[C@H]1CON(C)C1=O] | 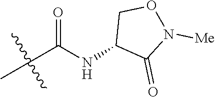 [SMILES: *C(=O)N[C@@H]1CON(C)C1=O] |
| 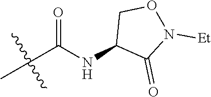 [SMILES: *C(=O)N[C@H]1CON(CC)C1=O] | 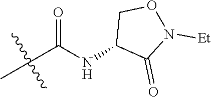 [SMILES: *C(=O)N[C@@H]1CON(CC)C1=O] |
| 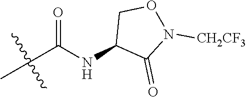 | 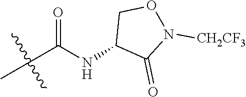 |
| 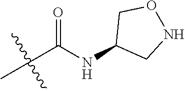 [SMILES: *C(=O)N[C@@H]1CNOC1] | 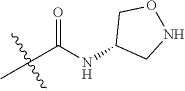 [SMILES: *C(=O)N[C@H]1CNOC1] |
| 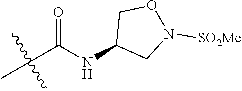 [SMILES: *C(=O)N[C@H]1CON(S(C)(=O)=O)C1] | 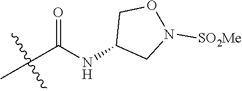 [SMILES: *C(=O)N[C@@H]1CON(S(C)(=O)=O)C1] |
| 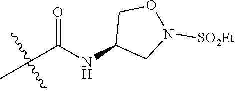 | 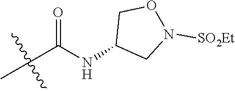 |
| 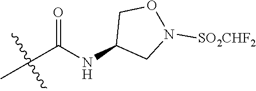 | 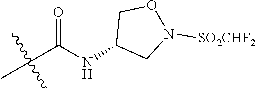 |
| 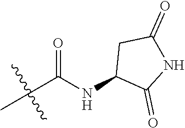 [SMILES: *C(=O)N[C@H]1CC(=O)NC1=O] | 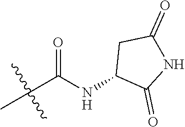 [SMILES: *C(=O)N[C@@H]1CC(=O)NC1=O] |
| 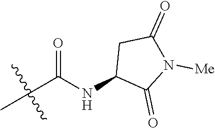 [SMILES: *C(=O)N[C@H]1CC(=O)N(C)C1=O] | 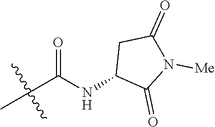 [SMILES: *C(=O)N[C@@H]1CC(=O)N(C)C1=O] |
| 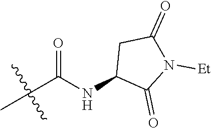 [SMILES: *C(=O)N[C@H]1CC(=O)N(CC)C1=O] | 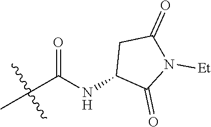 [SMILES: *C(=O)N[C@@H]1CC(=O)N(CC)C1=O] |
| 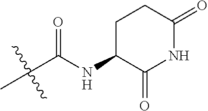 [SMILES: *C(=O)N[C@H]1CCC(=O)NC1=O] | 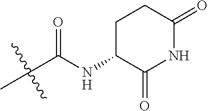 [SMILES: *C(=O)N[C@@H]1CCC(=O)NC1=O] |
| 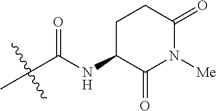 [SMILES: *C(=O)N[C@H]1CCC(=O)N(C)C1=O] | 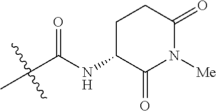 [SMILES: *C(=O)N[C@@H]1CCC(=O)N(C)C1=O] |
| 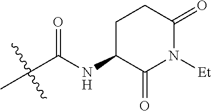 [SMILES: *C(=O)N[C@H]1CCC(=O)N(CC)C1=O] | 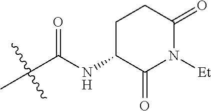 [SMILES: *C(=O)N[C@@H]1CCC(=O)N(CC)C1=O] |
| 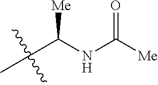 [SMILES: *[C@@H](C)NC(C)=O] | 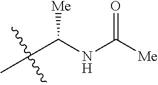 [SMILES: *[C@H](C)NC(C)=O] |
| 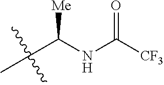 [SMILES: *[C@@H](C)NC(=O)C(F)(F)F] | 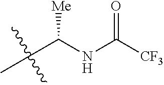 [SMILES: *[C@H](C)NC(=O)C(F)(F)F] |
| 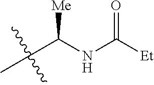 [SMILES: *[C@@H](C)NC(=O)CC] | 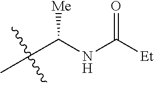 [SMILES: *[C@H](C)NC(=O)CC] |
| 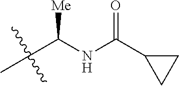 [SMILES: *[C@@H](C)NC(=O)C1CC1] | 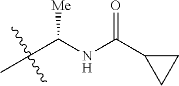 [SMILES: *[C@H](C)NC(=O)C1CC1] |
| 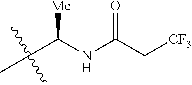 [SMILES: *[C@@H](C)NC(=O)CC(F)(F)F] | 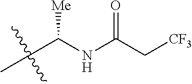 [SMILES: *[C@H](C)NC(=O)CC(F)(F)F] |

The present disclosure also includes Tables 2 through 142, each of which is constructed the same as Table 1 above, except that the row heading in Table 1 (i.e. “R¹ is Cl, R² is H, R³ is Cl, T is T-1 and X is O”) is replaced with the respective row headings shown below.

| Table | Row Heading |
|---|---|
| 2 | R¹ is Cl, R² is H, R³ is CF₃, T is T-1 and X is O. |
| 3 | R¹ is Cl, R² is F, R³ is CF₃, T is T-1 and X is O. |
| 4 | R¹ is Cl, R² is F, R³ is Cl, T is T-1 and X is O. |
| 5 | R¹ is H, R² is H, R³ is CF₃, T is T-1 and X is O. |
| 6 | R¹ is H, R² is F, R³ is CF₃, T is T-1 and X is O. |
| 7 | R¹ is H, R² is Cl, R³ is CF₃, T is T-1 and X is O. |
| 8 | R¹ is H, R² is Cl, R³ is Cl, T is T-1 and X is O. |
| 9 | R¹ is Cl, R² is H, R³ is Cl, T is T-1 and X is S. |
| 10 | R¹ is Cl, R² is H, R³ is CF₃, T is T-1 and X is S. |
| 11 | R¹ is Cl, R² is F, R³ is CF₃, T is T-1 and X is S. |
| 12 | R¹ is Cl, R² is F, R³ is Cl, T is T-1 and X is S. |
| 13 | R¹ is H, R² is H, R³ is CF₃, T is T-1 and X is S. |
| 14 | R¹ is H, R² is F, R³ is CF₃, T is T-1 and X is S. |
| 15 | R¹ is H, R² is Cl, R³ is CF₃, T is T-1 and X is S. |
| 16 | R¹ is H, R² is Cl, R³ is Cl, T is T-1 and X is S. |
| 17 | R¹ is Cl, R² is H, R³ is Cl, T is T-2 and X is O. |
| 18 | R¹ is Cl, R² is H, R³ is CF₃, T is T-2 and X is O. |
| 19 | R¹ is Cl, R² is F, R³ is CF₃, T is T-2 and X is O. |
| 20 | R¹ is Cl, R² is F, R³ is Cl, T is T-2 and X is O. |
| 21 | R¹ is H, R² is H, R³ is CF₃, T is T-2 and X is O. |
| 22 | R¹ is H, R² is F, R³ is CF₃, T is T-2 and X is O. |
| 23 | R¹ is H, R² is Cl, R³ is CF₃, T is T-2 and X is O. |
| 24 | R¹ is H, R² is Cl, R³ is Cl, T is T-2 and X is O. |
| 25 | R¹ is Cl, R² is H, R³ is Cl, T is T-2 and X is S. |
| 26 | R¹ is Cl, R² is H, R³ is CF₃, T is T-2 and X is S. |
| 27 | R¹ is Cl, R² is F, R³ is CF₃, T is T-2 and X is S. |
| 28 | R¹ is Cl, R² is F, R³ is Cl, T is T-2 and X is S. |
| 29 | R¹ is H, R² is H, R³ is CF₃, T is T-2 and X is S. |
| 30 | R¹ is H, R² is F, R³ is CF₃, T is T-2 and X is S. |
| 31 | R¹ is H, R² is Cl, R³ is CF₃, T is T-2 and X is S. |
| 32 | R¹ is H, R² is Cl, R³ is Cl, T is T-2 and X is S. |
| 33 | R¹ is Cl, R² is H, R³ is Cl, T is T-3 and X is O. |
| 34 | R¹ is Cl, R² is H, R³ is CF₃, T is T-3 and X is O. |
| 35 | R¹ is Cl, R² is F, R³ is CF₃, T is T-3 and X is O. |
| 36 | R¹ is Cl, R² is F, R³ is Cl, T is T-3 and X is O. |
| 37 | R¹ is H, R² is H, R³ is CF₃, T is T-3 and X is O. |
| 38 | R¹ is H, R² is F, R³ is CF₃, T is T-3 and X is O. |
| 39 | R¹ is H, R² is Cl, R³ is CF₃, T is T-3 and X is O. |
| 40 | R¹ is H, R² is Cl, R³ is Cl, T is T-3 and X is O. |
| 41 | R¹ is Cl, R² is H, R³ is Cl, T is T-3 and X is S. |
| 42 | R¹ is Cl, R² is H, R³ is CF₃, T is T-3 and X is S. |
| 43 | R¹ is Cl, R² is F, R³ is CF₃, T is T-3 and X is S. |
| 44 | R¹ is Cl, R² is F, R³ is Cl, T is T-3 and X is S. |
| 45 | R¹ is H, R² is H, R³ is CF₃, T is T-3 and X is S. |
| 46 | R¹ is H, R² is F, R³ is CF₃, T is T-3 and X is S. |
| 47 | R¹ is H, R² is Cl, R³ is CF₃, T is T-3 and X is S. |
| 48 | R¹ is H, R² is Cl, R³ is Cl, T is T-3 and X is S. |
| 49 | R¹ is Cl, R² is H, R³ is Cl, T is T-4 and X is O. |
| 50 | R¹ is Cl, R² is H, R³ is CF₃, T is T-4 and X is O. |
| 51 | R¹ is Cl, R² is F, R³ is CF₃, T is T-4 and X is O. |
| 52 | R¹ is Cl, R² is F, R³ is Cl, T is T-4 and X is O. |
| 53 | R¹ is H, R² is H, R³ is CF₃, T is T-4 and X is O. |
| 54 | R¹ is H, R² is F, R³ is CF₃, T is T-4 and X is O. |
| 55 | R¹ is H, R² is Cl, R³ is CF₃, T is T-4 and X is O. |
| 56 | R¹ is H, R² is Cl, R³ is Cl, T is T-4 and X is O. |
| 57 | R¹ is Cl, R² is H, R³ is Cl, T is T-4 and X is S. |
| 58 | R¹ is Cl, R² is H, R³ is CF₃, T is T-4 and X is S. |
| 59 | R¹ is Cl, R² is F, R³ is CF₃, T is T-4 and X is S. |
| 60 | R¹ is Cl, R² is F, R³ is Cl, T is T-4 and X is S. |
| 61 | R¹ is H, R² is H, R³ is CF₃, T is T-4 and X is S. |
| 62 | R¹ is H, R² is F, R³ is CF₃, T is T-4 and X is S. |
| 63 | R¹ is H, R² is Cl, R³ is CF₃, T is T-4 and X is S. |
| 64 | R¹ is H, R² is Cl, R³ is Cl, T is T-4 and X is S. |
| 65 | R¹ is Cl, R² is H, R³ is Cl, T is T-5 and X is O. |
| 66 | R¹ is Cl, R² is H, R³ is CF₃, T is T-5 and X is O. |
| 67 | R¹ is Cl, R² is F, R³ is CF₃, T is T-5 and X is O. |
| 68 | R¹ is Cl, R² is F, R³ is Cl, T is T-5 and X is O. |
| 69 | R¹ is H, R² is H, R³ is CF₃, T is T-5 and X is O. |
| 70 | R¹ is H, R² is F, R³ is CF₃, T is T-5 and X is O. |
| 71 | R¹ is H, R² is Cl, R³ is CF₃, T is T-5 and X is O. |
| 72 | R¹ is H, R² is Cl, R³ is Cl, T is T-5 and X is O. |
| 73 | R¹ is Cl, R² is H, R³ is Cl, T is T-5 and X is S. |
| 74 | R¹ is Cl, R² is H, R³ is CF₃, T is T-5 and X is S. |
| 75 | R¹ is Cl, R² is F, R³ is CF₃, T is T-5 and X is S. |
| 76 | R¹ is Cl, R² is F, R³ is Cl, T is T-5 and X is S. |
| 77 | R¹ is H, R² is H, R³ is CF₃, T is T-5 and X is S. |
| 78 | R¹ is H, R² is F, R³ is CF₃, T is T-5 and X is S. |
| 79 | R¹ is H, R² is Cl, R³ is CF₃, T is T-5 and X is S. |
| 80 | R¹ is H, R² is Cl, R³ is Cl, T is T-5 and X is S. |
| 81 | R¹ is Cl, R² is H, R³ is Cl, T is T-6 and X is O. |
| 82 | R¹ is Cl, R² is H, R³ is CF₃, T is T-6 and X is O. |
| 83 | R¹ is Cl, R² is F, R³ is CF₃, T is T-6 and X is O. |
| 84 | R¹ is Cl, R² is F, R³ is Cl, T is T-6 and X is O. |
| 85 | R¹ is H, R² is H, R³ is CF₃, T is T-6 and X is O. |
| 86 | R¹ is H, R² is F, R³ is CF₃, T is T-6 and X is O. |
| 87 | R¹ is H, R² is Cl, R³ is CF₃, T is T-6 and X is O. |
| 88 | R¹ is H, R² is Cl, R³ is Cl, T is T-6 and X is O. |
| 89 | R¹ is Cl, R² is H, R³ is Cl, T is T-6 and X is S. |
| 90 | R¹ is Cl, R² is H, R³ is CF₃, T is T-6 and X is S. |
| 91 | R¹ is Cl, R² is F, R³ is CF₃, T is T-6 and X is S. |
| 92 | R¹ is Cl, R² is F, R³ is Cl, T is T-6 and X is S. |
| 93 | R¹ is H, R² is H, R³ is CF₃, T is T-6 and X is S. |
| 94 | R¹ is H, R² is F, R³ is CF₃, T is T-6 and X is S. |
| 95 | R¹ is H, R² is Cl, R³ is CF₃, T is T-6 and X is S. |
| 96 | R¹ is H, R² is Cl, R³ is Cl, T is T-6 and X is S. |
| 97 | R¹ is Cl, R² is H, R³ is Cl, T is T-7 and X is O. |
| 98 | R¹ is Cl, R² is H, R³ is CF₃, T is T-7 and X is O. |
| 99 | R¹ is Cl, R² is F, R³ is CF₃, T is T-7 and X is O. |
| 100 | R¹ is Cl, R² is F, R³ is Cl, T is T-7 and X is O. |
| 101 | R¹ is H, R² is H, R³ is CF₃, T is T-7 and X is O. |
| 102 | R¹ is H, R² is F, R³ is CF₃, T is T-7 and X is O. |
| 103 | R¹ is H, R² is Cl, R³ is CF₃, T is T-7 and X is O. |
| 104 | R¹ is H, R² is Cl, R³ is Cl, T is T-7 and X is O. |
| 105 | R¹ is Cl, R² is H, R³ is Cl, T is T-7 and X is S. |
| 106 | R¹ is Cl, R² is H, R³ is CF₃, T is T-7 and X is S. |
| 107 | R¹ is Cl, R² is F, R³ is CF₃, T is T-7 and X is S. |
| 108 | R¹ is Cl, R² is F, R³ is Cl, T is T-7 and X is S. |
| 109 | R¹ is H, R² is H, R³ is CF₃, T is T-7 and X is S. |
| 110 | R¹ is H, R² is F, R³ is CF₃, T is T-7 and X is S. |
| 111 | R¹ is H, R² is Cl, R³ is CF₃, T is T-7 and X is S. |
| 112 | R¹ is H, R² is Cl, R³ is Cl, T is T-7 and X is S. |
| 113 | R¹ is Cl, R² is H, R³ is Cl, T is T-8 and X is O. |
| 114 | R¹ is Cl, R² is H, R³ is CF₃, T is T-8 and X is O. |
| 115 | R¹ is Cl, R² is F, R³ is CF₃, T is T-8 and X is O. |
| 116 | R¹ is Cl, R² is F, R³ is Cl, T is T-8 and X is O. |
| 117 | R¹ is H, R² is H, R³ is CF₃, T is T-8 and X is O. |
| 118 | R¹ is H, R² is F, R³ is CF₃, T is T-8 and X is O. |
| 119 | R¹ is H, R² is Cl, R³ is CF₃, T is T-8 and X is O. |
| 120 | R¹ is H, R² is Cl, R³ is Cl, T is T-8 and X is O. |
| 121 | R¹ is Cl, R² is H, R³ is Cl, T is T-8 and X is S. |
| 122 | R¹ is Cl, R² is H, R³ is CF₃, T is T-8 and X is S. |
| 123 | R¹ is Cl, R² is F, R³ is CF₃, T is T-8 and X is S. |
| 124 | R¹ is Cl, R² is F, R³ is Cl, T is T-8 and X is S. |
| 125 | R¹ is H, R² is H, R³ is CF₃, T is T-8 and X is S. |
| 126 | R¹ is H, R² is F, R³ is CF₃, T is T-8 and X is S. |
| 127 | R¹ is H, R² is Cl, R³ is CF₃, T is T-8 and X is S. |
| 128 | R¹ is H, R² is Cl, R³ is Cl, T is T-8 and X is S. |
| 129 | R¹ is Cl, R² is H, R³ is Cl and T is T-9. |
| 130 | R¹ is Cl, R² is H, R³ is CF₃ and T is T-9. |
| 131 | R¹ is Cl, R² is F, R³ is CF₃ and T is T-9. |
| 132 | R¹ is Cl, R² is F, R³ is Cl and T is T-9. |
| 133 | R¹ is H, R² is H, R³ is CF₃ and T is T-9. |
| 134 | R¹ is H, R² is F, R³ is CF₃ and T is T-9. |
| 135 | R¹ is H, R² is Cl, R³ is CF₃ and T is T-9. |
| 136 | R¹ is H, R² is Cl, R³ is Cl and T is T-9. |
| 137 | R¹ is Cl, R² is H, R³ is Cl and T is T-10. |
| 138 | R¹ is Cl, R² is H, R³ is CF₃ and T is T-10. |
| 139 | R¹ is Cl, R² is F, R³ is CF₃, T is T-10. |
| 140 | R¹ is Cl, R² is F, R³ is Cl and T is T-10. |
| 141 | R¹ is H, R² is H, R³ is CF₃ and T is T-10. |
| 142 | R¹ is H, R² is F, R³ is CF₃ and T is T-10. |
| 143 | R¹ is H, R² is Cl, R³ is CF₃ and T is T-10. |
| 144 | R¹ is H, R² is Cl, R³ is Cl and T is T-10. |

### Formulation/Utility

A compound of this disclosure will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil in water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil in water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

One way of dispensing the compositions disclosed herein over a target area, such as, but not limited to a crop-containing field, is by using drones. Use of drones or unmanned aerial vehicles (UAVs) in agricultural applications, such as for treating fields with chemical products, is rapidly expanding. A container of chemical products is coupled to the UAV and a material dispensing system mounted to the UAV, and the UAV is piloted above the area to be treated while the chemical product is dispensed.

The formulations will typically contain effective amounts of active ingredient, diluent and surfactant within the following approximate ranges which add up to 100 percent by weight.

### Weight Percent

| | Active | | |
|---|---|---|---|
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |

### Weight Percent

| | Active | | |
|---|---|---|---|
| | Ingredient | Diluent | Surfactant |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethylphosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters alkyl and aryl benzoates, y-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present disclosure often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this disclosure may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 m can be wet milled using media mills to obtain particles with average diameters below 3 m. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all formulations are prepared in conventional ways. Compound numbers refer to compounds in Index Table A. Without further elaboration, it is believed that one skilled in the art using the preceding description can utilize the present disclosure to its fullest extent. The following Examples are, therefore, to be construed as merely illustrative, and not limiting of the disclosure in any way whatsoever. Percentages are by weight except where otherwise indicated.

### Example A

### High Strength Concentrate

| | |
|---|---|
| Compound 24 | 98.5% |
| silica aerogel | 0.5% |
| synthetic amorphous fine silica | 1.0% |

### Example B

### Wettable Powder

| | |
|---|---|
| Compound 22 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

### Granule

| | |
|---|---|
| Compound 20 | 10.0% |
| attapulgite granules (low volatile matter, | 90.0% |
| 0.71/0.30 mm; U.S.S. No. 25-50 sieves) | |

### Example D

### Extruded Pellet

| | |
|---|---|
| Compound 18 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| Compound 16 | 10.0% |
| polyoxyethylene sorbitol hexoleate | 20.0% |
| C₆-C₁₀ fatty acid methyl ester | 70.0% |

### Example F

### Microemulsion

| | |
|---|---|
| Compound 14 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Seed Treatment

| | |
|---|---|
| Compound 12 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

### Fertilizer Stick

| | |
|---|---|
| Compound 10 | 2.5% |
| pyrrolidone-styrene copolymer | 4.8% |
| tristyrylphenyl 16-ethoxylate | 2.3% |
| talc | 0.8% |
| corn starch | 5.0% |
| slow-release fertilizer | 36.0% |
| kaolin | 38.0% |
| water | 10.6% |

### Example I

### Suspension Concentrate

| | |
|---|---|
| Compound 8 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

### Emulsion in Water

| | |
|---|---|
| Compound 6 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

### Oil Dispersion

| | |
|---|---|
| Compound 4 | 25% |
| polyoxyethylene sorbitol hexaoleate | 15% |
| organically modified bentonite clay | 2.5% |
| fatty acid methyl ester | 57.5% |

### Example L

### Suspoemulsion

| | |
|---|---|
| Compound 2 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum-based hydrocarbon | 20.0% |
| water | 53.7% |

Compounds of this disclosure exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, INVICTA RR2 PRO™, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may exhibit enhanced effects with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may exhibit enhanced effects with the phenotypic expression of proteins or other natural products toxic to invertebrate pests to provide greater-than-additive control of these pests.

Compositions of this disclosure can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present disclosure which further comprise at least one plant nutrient can be in the form of liquids or solids. Of note are solid formulations in the form of granules, small sticks or tablets. Solid formulations comprising a fertilizer composition can be prepared by mixing the compound or composition of the present disclosure with the fertilizer composition together with formulating ingredients and then preparing the formulation by methods such as granulation or extrusion. Alternatively solid formulations can be prepared by spraying a solution or suspension of a compound or composition of the present disclosure in a volatile solvent onto a previous prepared fertilizer composition in the form of dimensionally stable mixtures, e.g., granules, small sticks or tablets, and then evaporating the solvent.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Nonagronomic uses of the present compounds and compositions also include protecting human and animal health by controlling invertebrate pests that are parasitic or transmit infectious diseases. The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths. Compounds and compositions of the present disclosure are suitable for systemic and/or non-systemic control of infestation or infection by parasites on animals. Compounds and compositions of the present disclosure are particularly suitable for combating external parasitic or disease transmitting pests. Compounds and compositions of the present disclosure are suitable for combating parasites that infest agricultural working animals, such as cattle, sheep, goats, horses, pigs, donkeys, camels, buffalos, rabbits, hens, turkeys, ducks, geese and bees; pet animals and domestic animals such as dogs, cats, pet birds and aquarium fish; as well as so-called experimental animals, such as hamsters, guinea pigs, rats and mice. By combating these parasites, fatalities and performance reduction (in terms of meat, milk, wool, skins, eggs, honey, etc.) are reduced, so that applying a composition comprising a compound of the present disclosure allows more economic and simple husbandry of animals.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera frugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra brassicae Linnaeus), soybean looper (Pseudoplusia includens Walker), cabbage looper (Trichoplusia ni Hibner), tobacco budworm (Heliothis virescens Fabricius)); borers, casebearers, webworms, coneworms, cabbageworms and skeletonizers from the family Pyralidae (e.g., European corn borer (Ostrinia nubilalis Hubner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guen6e), green leafroller (Cnaphalocrocis medinalis), grape leaffolder (Desmia funeralis Hubner), melon worm (Diaphania nitidalis Stoll), cabbage center grub (Helluala hydralis Guen6e), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), striped riceborer (Chilo suppressalis Walker), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hubner), apple bud moth (Pandemis pyrusana Kearfott), omnivorous leafroller (Platynota stultana Walsingham), barred fruit-tree tortrix (Pandemis cerasana Hubner), apple brown tortrix (Pandemis heparana Denis & Schiffermüller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the order Hemiptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoridae and Delphacidae, treehoppers from the family Membracidae, psyllids from the families Liviidae, Psyllidae, and Triozidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borelhii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus FOrster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of the present disclosure also have activity on members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala perfoliata in horses, Fasciola hepatica Linnaeus in ruminants, etc.).

Compounds of the disclosure show particularly high activity against pests in the order Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hubner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermuller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hubner (cabbage looper) and Tuta absoluta Meyrick (tomato leafminer)).

Compounds of the disclosure also have significant activity on members from the order Hemiptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Passerini (rosy apple aphid), Eriosoma lanigerum Hausmann (woolly apple aphid), Hyalopterus pruni Geoffroy (mealy plum aphid), Lipaphis pseudobrassicae Davis (turnip aphid), Metopolophium dirhodum Walker (rose-grain aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricidus Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifoli Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrosteles quadrilineatus Forbes (aster leafhopper), Nephotettix cincticeps Uhler (green rice leafhopper), Nephotettix nigropictus Stål (rice leafhopper), Nilaparvata lugens Stål (brown planthopper), Peregrinus maidis Ashmead (corn planthopper), Sogatella furcifera Horvath (white-backed planthopper), Tagosodes orizicolus Muir (rice delphacid), Typhlocyba pomaria McAtee (white apple leafhopper), Erythroneura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this disclosure also have activity on members from the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schaffer (cotton stainer), Euchistus servus Say (brown stink bug), Euchistus variolarius Palisot de Beauvois (one-spotted stink bug), Graptosthetus spp. (complex of seed bugs), Halymorpha halys Stål (brown marmorated stink bug), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the disclosure include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Of note is use of compounds of this disclosure for controlling western flower thrip (Frankliniella occidentalis). Of note is use of compounds of this disclosure for controlling diamondback moth (Plutella xylostella). Of note is use of compounds of this disclosure for controlling fall armyworm (Spodoptera frugiperda).

Compounds of the present disclosure are also useful for increasing vigor of a crop plant. This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present disclosure can increase the vigor of treated plants compared to untreated plants by killing or otherwise preventing feeding of phytophagous invertebrate pests in the environment of the plants. In the absence of such control of phytophagous invertebrate pests, the pests reduce plant vigor by consuming plant tissues or sap, or transmitting plant pathogens such as viruses. Even in the absence of phytophagous invertebrate pests, the compounds of the disclosure may increase plant vigor by modifying metabolism of plants. Generally, the vigor of a crop plant will be most significantly increased by treating the plant with a compound of the disclosure if the plant is grown in a nonideal environment, i.e. an environment comprising one or more aspects adverse to the plant achieving the full genetic potential it would exhibit in an ideal environment.

Of note is the present method for increasing vigor of a crop plant wherein the crop plant is grown in an environment comprising phytophagous invertebrate pests. Also of note is the present method for increasing vigor of a crop plant wherein the crop plant is grown in an environment not comprising phytophagous invertebrate pests. Also of note is the present method for increasing vigor of a crop plant wherein the crop plant is grown in an environment comprising an amount of moisture less than ideal for supporting growth of the crop plant. Of note is the present method for increasing vigor of a crop plant wherein the crop is rice. Also of note is the present method for increasing vigor of a crop plant wherein the crop is maize (corn). Also of note is the present method for increasing vigor of a crop plant wherein the crop is soybean.

Compounds of this disclosure can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus the present disclosure also pertains to a composition comprising a biologically effective amount of a compound of Formula 1, at least one additional component selected from the group consisting of surfactants, solid diluents and liquid diluents, and at least one additional biologically active compound or agent. For mixtures of the present disclosure, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this disclosure can be formulated are insecticides such as abamectin, acephate, acequinocyl, acetamiprid, acrinathrin, acynonapyr, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorantraniliprole, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cyantraniliprole, (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)˜1B-pyrazole-5-carboxamide), cycloprothrin, cycloxaprid ((5S,8R)-1-[(6-chloro-3-pyidinyl)methyl]-2,3,5,6,7,8-hexabydro-9-nitro-5,8-Epoxy-1H-inidazo[1,2-a]azepine), eyenopyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromesotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, fluazaindolizine, flubendiamide, flucythrinate, flufenerim, flufenoxuron, flufenoxystrobin (methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), flupyrimin, fluvalinate, tau-fluvalinate, fluxametamide, fonophos, formetanate, fosthiazate, gamma-cyhalothrin, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, oxamyl, oxazosulfyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spiropidion, spirotetramat, sulprofos, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, kappa-tefluthrin, terbufos, tetrachlorantraniliprole, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, tyclopyrazoflor, zeta-cypermethrin, Bacillus thuringiensis delta-endotoxins, entomopathogenic bacteria, entomopathogenic viruses or entomopathogenic fungi.

Of note are insecticides such as abamectin, acetamiprid, acrinathrin, acynonapyr, afidopyropen, amitraz, avermectin, azadirachtin, benfuracarb, bensultap, bifenthrin, buprofezin, broflanilide, cadusafos, carbaryl, cartap, chlorantraniliprole, chloroprallethrin, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, epsilon-metofluthrin, esfenvalerate, ethiprole, etofenprox, etoxazole, fenitrothion, fenothiocarb, fenoxycarb, fenvalerate, fipronil, flometoquin, fluxametamide, flonicamid, flubendiamide, fluensulfone, flufenoxuron, flufenoxystrobin, flufensulfone, flupiprole, flupyrimin, flupyradifurone, fluvalinate, formetanate, fosthiazate, gamma-cyhalothrin, heptafluthrin, hexaflumuron, hydramethylnon, imidacloprid, indoxacarb, isocycloseram, kappa-tefluthrin, lambda-cyhalothrin, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, tyclopyrazoflor, zeta-cypermethrin, Bacillus thuringiensis delta-endotoxins, all strains of Bacillus thuringiensis and all strains of nucleo polyhedrosis viruses.

One embodiment of biological agents for mixing with compounds of this disclosure include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Indiana, USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydiapomonella granulosis virus (CpGV).

One embodiment of biological agents for mixing with compounds of this disclosure include one or a combination of (i) a bacterium of the genus Actinomycetes, Agrobacterium, Arthrobacter, Alcaligenes, Aureobacterium, Azobacter, Bacillus, Beijerinckia, Bradyrhizobium, Brevibacillus, Burkholderia, Chromobacterium, Clostridium, Clavibacter, Comamonas, Corynebacterium, Curtobacterium, Enterobacter, Flavobacterium, Gluconobacter, Hydrogenophaga, Klebsiella, Methylobacterium, Paenibacillus, Pasteuria, Photorhabdus, Phyllobacterium, Pseudomonas, Rhizobium, Serratia, Sphingobacterium, Stenotrophomonas, Streptomyces, Variovorax, or Xenorhabdus, for example a bacterium of Bacillus amyloliquefaciens, Bacillus cereus, Bacillus firmus, Bacillus, lichenformis, Bacillus pumilus, Bacillus sphaericus, Bacillus subtilis, Bacillus thuringiensis, Bradyrhizobium japonicum, Chromobacterium subtsugae, Pasteuria nishizawae, Pasteuria penetrans, Pasteuria usage, Pseudomonas fluorescens, and Streptomyces lydicus; (ii) a fungus such as green muscardine fungus; (iii) a virus including baculovirus, nucleopolyhedro virus such as Helicoverpa zea nucleopolyhedrovirus, Anagrapha falcifera nucleopolyhedrovirus; granulosis virus such as Cydia pomonella granulosis virus.

Of particular note is such a combination where the other invertebrate pest control active ingredient belongs to a different chemical class or has a different site of action than compounds of Formula 1. In certain instances, a combination with at least one other invertebrate pest control active ingredient having a similar spectrum of control but a different site of action will be particularly advantageous for resistance management. Thus, a composition of the present disclosure can further comprise a biologically effective amount of at least one additional invertebrate pest control active ingredient having a similar spectrum of control but belonging to a different chemical class or having a different site of action. These additional biologically active compounds or agents include, but are not limited to, acetylcholinesterase (AChE) inhibitors such as the carbamates methomyl, oxamyl, thiodicarb, triazamate, and the organophosphates chlorpyrifos; GABA-gated chloride channel antagonists such as the cyclodienes dieldrin and endosulfan, and the phenylpyrazoles ethiprole and fipronil; sodium channel modulators such as the pyrethroids bifenthrin, cyfluthrin, beta-cyfluthrin, cyhalothrin, lambda-cyhalothrin, cypermethrin, deltamethrin, dimefluthrin, esfenvalerate, metofluthrin and profluthrin; nicotinic acetylcholinereceptor (nAChR) agonists such as the neonicotinoids acetamiprid, clothianidin, dinotefuran, imidacloprid, nitenpyram, nithiazine, thiacloprid, and thiamethoxam, and sulfoxaflor; nicotinic acetylcholine receptor (nAChR) allosteric activators such as the spinosyns spinetoram and spinosad; chloride channel activators such as the avermectins abamectin and emamectin; juvenile hormone mimics such as diofenolan, methoprene, fenoxycarb and pyriproxyfen; selective homopteran feeding blockers such as pymetrozine and flonicamid; mite growth inhibitors such as etoxazole; inhibitors of mitochondrial ATP synthase such as propargite; ucouplers of oxidative phosphorylation via disruption of the proton gradient such as chlorfenapyr; nicotinic acetylcholine receptor (nAChR) channel blockers such as the nereistoxin analogs cartap; inhibitors of chitin biosynthesis such as the benzoylureas flufenoxuron, hexaflumuron, lufenuron, novaluron, noviflumuron and triflumuron, and buprofezin; dipteran moulting disrupters such as cyromazine; ecdysone receptor agonists such as the diacylhydrazines methoxyfenozide and tebufenozide; octopamine receptor agonists such as amitraz; mitochondrial complex III electron transport inhibitors such as hydramethylnon; mitochondrial complex I electron transport inhibitors such as pyridaben; voltage-dependent sodium channel blockers such as indoxacarb; inhibitors of acetyl CoA carboxylase such as the tetronic and tetramic acids spirodiclofen, spiromesifen and spirotetramat; mitochondrial complex II electron transport inhibitors such as the 8-ketonitriles cyenopyrafen and cyflumetofen; ryanidine receptor modulators such as the anthranilic diamides chlorantraniliprole, cyantraniliprole and cyantraniliprole, diamides such as flubendiamide, and ryanodine receptor ligands such as ryanodine; compounds wherein the target site responsible for biological activity is unknown or uncharacterized such as azadirachtin, bifenazate, pyridalyl, pyrifluquinazon and triflumezopyrim; microbial disrupters of insect midgut membranes such as Bacillus thuringensis and the delta-endotoxins they produce and Bacillus sphaericus; and biological agents including nucleo polyhedro viruses (NPV) and other naturally occurring or genetically modified insecticidal viruses.

Further examples of biologically active compounds or agents with which compounds of this disclosure can be formulated are: fungicides such as acibenzolar-S-methyl, aldimorph, ametoctradin, aminopyrifen, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlobentiazox, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dipymetitrone, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpicoxamid, fenpropidin, fenpropimorph, fenpyrazamine, fentin acetate, fentin hydroxide, ferbam, ferimzone, flometoquin, florylpicoxamid, fluopimomide, fluazinam, fludioxonil, flufenoxystrobin, fluindapyr, flumorph, fluopicolide, fluopyram, fluoxapiprolin, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, inpyrfluxam, iodicarb, ipconazole, ipfentrifluconazole, ipflufenoquin, isofetamid, iprobenfos, iprodione, iprovalicarb, isoflucypram, isoprothiolane, isopyrazam, isotianil, kasugamycin, kresoxim-methyl, lancotrione, mancozeb, mandipropamid, mandestrobin, maneb, mapanipyrin, mefentrifluconazole, mepronil, meptyldinocap, metalaxyl (including metalaxyl-M/mefenoxam), metconazole, methasulfocarb, metiram, metominostrobin, metyltetraprole, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pydiflumnetoten (Adepidyn®), pyraclostrobin, pyrametostrobin, pyrapropoyne, pyraoxystrobin, pyraziflumid, pyrazophos, pyribencarb, pyributacarb, pyridachlometyl, pyrifenox, pyriofenone, perisoxazole, pyrimethanil, pyrifenox, pyrrolnitrin, pyroquilon, quinconazole, quinmethionate, quinofumelin, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide and 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone; nematocides such as fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus and Pasteuria nishizawae; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

In certain instances, combinations of a compound of this disclosure with other biologically active (particularly invertebrate pest control) compounds or agents (i.e. active ingredients) can result in an enhanced effect. Reducing the quantity of active ingredients released in the environment while ensuring effective pest control is always desirable. When enhanced invertebrate pest control occurs at application rates giving agronomically satisfactory levels of invertebrate pest control, such combinations can be advantageous for reducing crop production cost and decreasing environmental load.

Compounds of this disclosure and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The exogenously applied invertebrate pest control compounds of this disclosure in combination with the expressed toxin proteins may provide an enhanced effect.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001.

Compounds of this disclosure can be combined or formulated with polynucleotides including, but not limited to, DNA, RNA, and/or chemically modified nucleotides influencing the amount of a particular target through down regulation, interference, suppression or silencing of the genetically derived transcript that render an insecticidal effect.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components can expand the spectrum of invertebrate pests controlled beyond the spectrum controlled by the compound of Formula 1 alone.

Invertebrate pests are controlled in agronomic and nonagronomic applications by applying one or more compounds of this disclosure, typically in the form of a composition, in a biologically effective amount, to the environment of the pests, including the agronomic and/or nonagronomic locus of infestation, to the area to be protected, or directly on the pests to be controlled.

Thus the present disclosure comprises a method for controlling an invertebrate pest in agronomic and/or nonagronomic applications, comprising contacting the invertebrate pest or its environment with a biologically effective amount of one or more of the compounds of the disclosure, or with a composition comprising at least one such compound or a composition comprising at least one such compound and a biologically effective amount of at least one additional biologically active compound or agent. Examples of suitable compositions comprising a compound of the disclosure and a biologically effective amount of at least one additional biologically active compound or agent include granular compositions wherein the additional active compound is present on the same granule as the compound of the disclosure or on granules separate from those of the compound of the disclosure.

To achieve contact with a compound or composition of the disclosure to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the disclosure can be applied to the plant foliage or the soil. Compounds of this disclosure can also be effectively delivered through plant uptake by contacting the plant with a composition comprising a compound of this disclosure applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a compound of the present disclosure or with a composition comprising a biologically effective amount of a compound of the present disclosure. Of further note is this method wherein the environment is soil and the composition is applied to the soil as a soil drench formulation. Of further note is that compounds of this disclosure are also effective by localized application to the locus of infestation. Other methods of contact include application of a compound or a composition of the disclosure by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the disclosure. The compounds of this disclosure can also be impregnated into materials for fabricating invertebrate control devices (e.g., insect netting).

Compounds of the disclosure are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant and seed cultivars which can be treated according to the disclosure include those that are resistant against one or more biotic stresses (pests such as nematodes, insects, mites, fungi, etc.) or abiotic stresses (drought, cold temperature, soil salinity, etc.), or that contain other desirable characteristics. Plants and seeds can be genetically modified to exhibit traits of, for example, herbicide tolerance, insect-resistance, modified oil profiles or drought tolerance. Useful genetically modified plants and seeds containing single gene transformation events or combinations of transformation events are listed in Table Z. Additional information for the genetic modifications listed in Table Z can be obtained from the following databases:
OECD BioTrack Product Database [database online]. Retrieved from The Organisation for Economic Co-operation and Development (OECD) using internet <https://biotrackproductdatabase.oecd.org/byidentifier.aspx>
USDA Animal and Plant Health Inspection Service [database online]. Retrieved from the US Department of Agriculture using the internet <http://www.aphis.usda.gov>
Deliberate Release and Placing on the EU Market of GMOs—GMO Register [database online]. Retrieved from the European Commission Joint Research Centre using internet <http://gmoinfo.jrc.ec.europa.eu>

The following abbreviations are used in Table Z which follows: tol. is tolerance, res. is resistance, SU is sulfonylurea, ALS is acetolactate synthase, HPPD is 4-Hydroxyphenylpyruvate Dioxygenase, NA is Not Available?

TABLE Z

| Crop | Event Name | Event Code | Trait(s) | Gene(s) |
|---|---|---|---|---|
| Alfalfa | J101 | MON-00101-8 | Glyphosate tol. | cp4 epsps (aroA:CP4) |
| Alfalfa | J163 | MON-ØØ163-7 | Glyphosate tol. | cp4 epsps (aroA:CP4) |
| Canola* | 23-18-17 | CGN-89465-2 | High lauric | te |
| | (Event 18) | | acid oil | |
| Canola* | 23-198 | CGN-89465-2 | High lauric | te |
| | (Event 23) | | acid oil | |
| Canola* | 61061 | DP-Ø61Ø61-7 | Glyphosate tol. | gat4621 |
| Canola* | 73496 | DP-Ø73496-4 | Glyphosate tol. | gat4621 |
| Canola* | GT200 (RT200) | MON-89249-2 | Glyphosate tol. | cp4 epsps |
| | | | | (aroA:CP4); goxv247 |
| Canola* | GT73 (RT73) | MON-ØØØ73-7 | Glyphosate tol. | cp4 epsps |
| | | | | (aroA:CP4); goxv247 |
| Canola* | HCN10 | NA | Glufosinate tol. | bar |
| | (Topas 19/2) | | | |
| Canola* | HCN28 (T45) | ACS-BNØØ8-2 | Glufosinate tol. | pat (syn) |
| Canola* | HCN92 | ACS-BNØØ7-1 | Glufosinate tol. | bar |
| | (Topas 19/2) | | | |
| Canola* | MON88302 | MON-883Ø2-9 | Glyphosate tol. | cp4 epsps (aroA:CP4) |
| Canola* | MPS961 | NA | Phytate breakdown | phyA |
| Canola* | MPS962 | NA | Phytate breakdown | phyA |
| Canola* | MPS963 | NA | Phytate breakdown | phyA |
| Canola* | MPS964 | NA | Phytate breakdown | phyA |
| Canola* | MPS965 | NA | Phytate breakdown | phyA |
| Canola* | MS1 (B91-4) | ACS-BNØØ4-7 | Glufosinate tol. | bar |
| Canola* | MS8 | ACS-BNØØ5-8 | Glufosinate tol. | bar |
| Canola* | OXY-235 | ACS-BNØ11-5 | Oxynil tol. | bxn |
| Canola* | PHY14 | NA | Glufosinate tol. | bar |
| Canola* | PHY23 | NA | Glufosinate tol. | bar |
| Canola* | PHY35 | NA | Glufosinate tol. | bar |
| Canola* | PHY36 | NA | Glufosinate tol. | bar |
| Canola* | RF1 (B93-101) | ACS-BNØØ1-4 | Glufosinate tol. | bar |
| Canola* | RF2 (B94-2) | ACS-BNØØ2-5 | Glufosinate tol. | bar |
| Canola* | RF3 | ACS-BNØØ3-6 | Glufosinate tol. | bar |
| Bean | EMBRAPA 5.1 | EMB-PV051-1 | Disease res | ac1 (sense and |
| | | | | antisense) |
| Brinjal | EE-1 | | Insect res | cry1Ac |
| (Eggplant) | | | | |
| Carnation | 11 (7442) | FLO-07442-4 | SU tol.; modified | surB; dfr; |
| | | | flower color | hfl (f3′5′h) |
| Carnation | 11363 (1363A) | FLO-11363-1 | SU tol.; modified | surB; dfr; |
| | | | flower color | bp40 (f3′5′h) |
| Carnation | 1226A (11226) | FLO-11226-8 | SU tol.; modified | surB; dfr; |
| | | | flower color | bp40 (f3′5′h) |
| Carnation | 123.2.2 (40619) | FLO-4Ø619-7 | SU tol.; modified | surB; dfr; |
| | | | flower color | hfl (f3′5′h) |
| Carnation | 123.2.38 (40644) | FLO-4Ø644-4 | SU tol.; modified | surB; dfr; |
| | | | flower color | hfl (f3′5′h) |
| Carnation | 123.8.12 | FLO-4Ø689-6 | SU tol.; modified | surB; dfr; |
| | | | flower color | bp40 (f3′5′h) |
| Carnation | 123.8.8 (40685) | FLO-4Ø685-1 | SU tol.; modified | surB; dfr; |
| | | | flower color | bp40 (f3′5′h) |
| Carnation | 1351A (11351) | FLO-11351-7 | SU tol.; modified | surB; dfr; |
| | | | flower color | bp40 (f3′5′h) |
| Carnation | 1400A (11400) | FLO-114ØØ-2 | SU tol.; modified | surB; dfr; |
| | | | flower color | bp40 (f3′5′h) |
| Carnation | 15 | FLO-ØØØ15-2 | SU tol.; modified | surB; dfr; |
| | | | flower color | hfl (f3′5′h) |
| Carnation | 16 | FLO-ØØØ16-3 | SU tol.; modified | surB; dfr; |
| | | | flower color | hfl (f3′5′h) |
| Carnation | 4 | FLO-ØØØØ4-9 | SU tol.; modified | surB; dfr; |
| | | | flower color | hfl (f3′5′h) |
| Carnation | 66 | FLO-ØØØ66-8 | SU tol.; delayed | surB; acc |
| | | | senescence | |
| Carnation | 959A (11959) | FLO-11959-3 | SU tol.; modified | surB; dfr; |
| | | | flower color | bp40 (f3′5′h) |
| Carnation | 988A (11988) | FLO-11988-7 | SU tol.; modified | surB; dfr; |
| | | | flower color | bp40 (f3′5′h) |
| Carnation | 26407 | IFD-26497-2 | SU tol.; modified | surB; dfr; |
| | | | flower color | bp40 (f3′5′h) |
| Carnation | 25958 | IFD-25958-3 | SU tol.; modified | surB; dfr; |
| | | | flower color | bp40 (f3′5′h) |
| Chicory | RM3-3 | NA | Glufosinate tol. | bar |
| Chicory | RM3-4 | NA | Glufosinate tol. | bar |
| Chicory | RM3-6 | NA | Glufosinate tol. | bar |
| Cotton | 19-51a | DD-Ø1951A-7 | ALS herbicide tol. | S4-HrA |
| Cotton | 281-24-236 | DAS-24236-5 | Glufosinate tol.; | pat (syn); |
| | | | insect res. | cry1F |
| Cotton | 3006-210-23 | DAS-21Ø23-5 | Glufosinate tol.; | pat (syn); |
| | | | insect res. | cry1Ac |
| Cotton | 31707 | NA | Oxynil tol.; | bxn; cry1Ac |
| | | | insect res. | |
| Cotton | 31803 | NA | Oxynil tol.; | bxn; cry1Ac |
| | | | insect res. | |
| Cotton | 31807 | NA | Oxynil tol.; | bxn; cry1Ac |
| | | | insect res. | |
| Cotton | 31808 | NA | Oxynil tol.; | bxn; cry1Ac |
| | | | insect res. | |
| Cotton | 42317 | NA | Oxynil tol.; | bxn; cry1Ac |
| | | | insect res. | |
| Cotton | BNLA-601 | NA | Insect res. | cry1Ac |
| Cotton | BXN10211 | BXN10211-9 | Oxynil tol. | bxn; cry1Ac |
| Cotton | BXN10215 | BXN10215-4 | Oxynil tol. | bxn; cry1Ac |
| Cotton | BXN10222 | BXN10222-2 | Oxynil tol. | bxn; cry1Ac |
| Cotton | BXN10224 | BXN10224-4 | Oxynil tol. | bxn; cry1Ac |
| Cotton | COT102 | SYN-IR102-7 | Insect res. | vip3A(a) |
| Cotton | COT67B | SYN-IR67B-1 | Insect res. | cry1Ab |
| Cotton | COT202 | | Insect res. | vip3A |
| Cotton | Event 1 | NA | Insect res. | cry1Ac |
| Cotton | GMF Cry1A | GTL-GMF311-7 | Insect res. | cry1Ab-Ac |
| Cotton | GHB119 | BCS-GH005-8 | Insect res. | cry2Ae |
| Cotton | GHB614 | BCS-GH002-5 | Glyphosate tol. | 2mepsps |
| Cotton | GK12 | NA | Insect res. | cry1Ab-Ac |
| Cotton | LLCotton25 | ACS-GH001-3 | Glufosinate tol. | bar |
| Cotton | MLS 9124 | NA | Insect res. | cry1C |
| Cotton | MON1076 | MON-89924-2 | Insect res. | cry1Ac |
| Cotton | MON1445 | MON-01445-2 | Glyphosate tol. | cp4 epsps (aroA:CP4) |
| Cotton | MON15985 | MON-15985-7 | Insect res. | cry1Ac; cry2Ab2 |
| Cotton | MON1698 | MON-89383-1 | Glyphosate tol. | cp4 epsps (aroA:CP4) |
| Cotton | MON531 | MON-00531-6 | Insect res. | cry1Ac |
| Cotton | MON757 | MON-00757-7 | Insect res. | cry1Ac |
| Cotton | MON88913 | MON-88913-8 | Glyphosate tol. | cp4 epsps (aroA:CP4) |
| Cotton | Nqwe Chi 6 Bt | NA | Insect res. | NA? |
| Cotton | SKG321 | NA | Insect res. | cry1A; CpTI |
| Cotton | T303-3 | BCS-GH003-6 | Insect res.; | cry1Ab; bar |
| | | | glufosinate tol. | |
| Cotton | T304-40 | BCS-GH004-7 | Insect res.; | cry1Ab; bar |
| | | | glufosinate tol. | |
| Cotton | CE43-67B | | Insect res. | cry1Ab |
| Cotton | CE46-02A | | Insect res. | cry1Ab |
| Cotton | CE44-69D | | Insect res. | cry1Ab |
| Cotton | 1143-14A | | Insect res. | cry1Ab |
| Cotton | 1143-51B | | Insect res. | cry1Ab |
| Cotton | T342-142 | | Insect res. | cry1Ab |
| Cotton | PV-GHGT07 | | Glyphosate tol. | cp4 epsps (aroA:CP4) |
| | (1445) | | | |
| Cotton | EE-GH3 | | Glyphosate tol. | mepsps |
| Cotton | EE-GH5 | | Insect res. | cry1Ab |
| Cotton | MON88701 | MON-88701-3 | Dicamba & | Modified dmo; bar |
| | | | glufosinate tol. | |
| Cotton | OsCr11 | | Anti-allergy | Modified Cry j |
| Creeping | ASR368 | SMG-368ØØ-2 | Glyphosate tol. | cp4 epsps (aroA:CP4) |
| Bentgrass | | | | |
| Eucalyptus | 20-C | | Salt tol. | codA |
| Eucalyptus | 12-5C | | Salt tol. | codA |
| Eucalyptus | 12-5B | | Salt tol. | codA |
| Eucalyptus | 107-1 | | Salt tol. | codA |
| Eucalyptus | Jan. 9, 2001 | | Salt tol. | codA |
| Eucalyptus | Feb. 1, 2001 | | Salt tol. | codA |
| Eucalyptus | | | Cold tol. | des9 |
| Flax | FP967 | CDC-FL001-2 | ALS herbicide tol. | als |
| Lentil | RH44 | | Imidazolinone tol. | als |
| Maize | 3272 | SYN-E3272-5 | Modified alpha-amylase | amy797E |
| Maize | 5307 | SYN-05307-1 | Insect res. | ecry3.1Ab |
| Maize | 59122 | DAS-59122-7 | Insect res.; | cry34Ab1; |
| | | | glufosinate tol. | cry35Ab1; pat |
| Maize | 676 | PH-000676-7 | Glufosinate tol.; | pat; dam |
| | | | pollination control | |
| Maize | 678 | PH-000678-9 | Glufosinate tol.; | pat; dam |
| | | | pollination control | |
| Maize | 680 | PH-000680-2 | Glufosinate tol.; | pat; dam |
| | | | pollination control | |
| Maize | 98140 | DP-098140-6 | Glyphosate toll; ALS | gat4621; zm-hra |
| | | | herbicide tol. | |
| Maize | Bt10 | NA | Insect res.; | cry1Ab; pat |
| | | | glufosinate tol. | |
| Maize | Bt176 (176) | SYN-EV176-9 | Insect res.; | cry1Ab; bar |
| | | | glufosinate tol. | |
| Maize | BVLA430101 | NA | Phytate breakdown | phyA2 |
| Maize | CBH-351 | ACS-ZM004-3 | Insect res.; | cry9C; bar |
| | | | glufosinate tol. | |
| Maize | DAS40278-9 | DAS40278-9 | 2,4-D tol. | aad-1 |
| Maize | DBT418 | DKB-89614-9 | Insect res.; | cry1Ac; pinII; bar |
| | | | glufosinate tol. | |
| Maize | DLL25 (B16) | DKB-89790-5 | Glufosinate tol. | bar |
| Maize | GA21 | MON-00021-9 | Glyphosate tol. | mepsps |
| Maize | GG25 | | Glyphosate tol. | mepsps |
| Maize | GJ11 | | Glyphosate tol. | mepsps |
| Maize | Fl117 | | Glyphosate tol. | mepsps |
| Maize | GAT-ZM1 | | Glufosinate tol. | pat |
| Maize | LY038 | REN-00038-3 | Increased lysine | cordapA |
| Maize | MIR162 | SYN-IR162-4 | Insect res. | vip3Aa20 |
| Maize | MIR604 | SYN-IR604-5 | Insect res. | mcry3A |
| Maize | MON801 | MON801 | Insect res.; | cry1Ab; cp4 epsps |
| | (MON80100) | | glyphosate tol. | (aroA:CP4); goxv247 |
| Maize | MON802 | MON-80200-7 | Insect res.; | cry1Ab; cp4 epsps |
| | | | glyphosate tol. | (aroA:CP4); goxv247 |
| Maize | MON809 | PH-MON-809-2 | Insect res.; | cry1Ab; cp4 epsps |
| | | | glyphosate tol. | (aroA:CP4); goxv247 |
| Maize | MON810 | MON-00810-6 | Insect res.; | cry1Ab; cp4 epsps |
| | | | glyphosate tol. | (aroA:CP4); goxv247 |
| Maize | MON832 | NA | Glyphosate tol. | cp4 epsps |
| | | | | (aroA:CP4); goxv247 |
| Maize | MON863 | MON-00863-5 | Insect res. | cry3Bb1 |
| Maize | MON87427 | MON-87427-7 | Glyphosate tol. | cp4 epsps (aroA:CP4) |
| Maize | MON87460 | MON-87460-4 | Drought tol. | cspB |
| Maize | MON88017 | MON-88017-3 | Insect res.; | cry3Bb1; cp4 |
| | | | glyphosate tol. | epsps (aroA:CP4) |
| Maize | MON89034 | MON-89034-3 | Insect res. | cry2Ab2; cry1A.105 |
| Maize | MS3 | ACS-ZM001-9 | Glufosinate tol.; | bar; barnase |
| | | | pollination control | |
| Maize | MS6 | ACS-ZM005-4 | Glufosinate tol.; | bar; barnase |
| | | | pollination control | |
| Maize | NK603 | MON-00603-6 | Glyphosate tol. | cp4 epsps (aroA:CP4) |
| Maize | T14 | ACS-ZM002-1 | Glufosinate tol. | pat (syn) |
| Maize | T25 | ACS-ZM003-2 | Glufosinate tol. | pat (syn) |
| Maize | TC1507 | DAS-01507-1 | Insect res.; | cry1Fa2; pat |
| | | | glufosinate tol. | |
| Maize | TC6275 | DAS-06275-8 | Insect res.; | mocry1F; bar |
| | | | glufosinate tol. | |
| Maize | VIP1034 | | Insect res.; | vip3A; pat |
| | | | glufosinate tol. | |
| Maize | 43A47 | DP-043A47-3 | Insect res.; | cry1F; cry34Ab1; |
| | | | glufosinate tol. | cry35Ab1; pat |
| Maize | 40416 | DP-040416-8 | Insect res.; | cry1F; cry34Ab1; |
| | | | glufosinate tol. | cry35Ab1; pat |
| Maize | 32316 | DP-032316-8 | Insect res.; | cry1F; cry34Ab1; |
| | | | glufosinate tol. | cry35Ab1; pat |
| Maize | 4114 | DP-004114-3 | Insect res.; | cry1F; cry34Ab1; |
| | | | glufosinate tol. | cry35Ab1; pat |
| Melon | Melon A | NA | Delayed | sam-k |
| | | | ripening/senescence | |
| Melon | Melon B | NA | Delayed | sam-k |
| | | | ripening/senescence | |
| Papaya | 55-1 | CUH-CP551-8 | Disease res. | prsv cp |
| Papaya | 63-1 | CUH-CP631-7 | Disease res. | prsv cp |
| Papaya | Huanong No. 1 | NA | Disease res. | prsv rep |
| Papaya | X17-2 | UFL-X17CP-6 | Disease res. | prsv cp |
| Petunia | Petunia-CHS | NA | Modified product quality | CHS suppres.sion |
| Plum | C-5 | ARS-PLMC5-6 | Disease res. | ppv cp |
| Canola** | ZSR500 | NA | Glyphosate tol. | cp4 epsps |
| | | | | (aroA:CP4); goxv247 |
| Canola** | ZSR502 | NA | Glyphosate tol. | cp4 epsps |
| | | | | (aroA:CP4); goxv247 |
| Canola** | ZSR503 | NA | Glyphosate tol. | cp4 epsps |
| | | | | (aroA:CP4); goxv247 |
| Poplar | Bt poplar | NA | Insect res. | cry1Ac; API |
| Poplar | Hybrid poplar | NA | Insect res. | cry1Ac; API |
| | clone 741 | | | |
| Poplar | trg300-1 | | High cellulose | AaXEG2 |
| Poplar | trg300-2 | | High cellulose | AaXEG2 |
| Potato | 1210 amk | NA | Insect res. | cry3A |
| Potato | 2904/1 kgs | NA | Insect res. | cry3A |
| Canola** | ZSR500 | NA | Glyphosate tol. | cp4 epsps |
| | | | | (aroA:CP4); goxv247 |
| Canola** | ZSR502 | NA | Glyphosate tol. | cp4 epsps |
| | | | | (aroA:CP4); goxv247 |
| Potato | ATBT04-27 | NMK-89367-8 | Insect res. | cry3A |
| Potato | ATBT04-30 | NMK-89613-2 | Insect res. | cry3A |
| Potato | ATBT04-31 | NMK-89170-9 | Insect res. | cry3A |
| Potato | ATBT04-36 | NMK-89279-1 | Insect res. | cry3A |
| Potato | ATBT04-6 | NMK-89761-6 | Insect res. | cry3A |
| Potato | BT06 | NMK-89812-3 | Insect res. | cry3A |
| Potato | BT10 | NMK-89175-5 | Insect res. | cry3A |
| Potato | BT12 | NMK-89601-8 | Insect res. | cry3A |
| Potato | BT16 | NMK-89167-6 | Insect res. | cry3A |
| Potato | BT17 | NMK-89593-9 | Insect res. | cry3A |
| Potato | BT18 | NMK-89906-7 | Insect res. | cry3A |
| Potato | BT23 | NMK-89675-1 | Insect res. | cry3A |
| Potato | EH92-527-1 | BPS-25271-9 | Modified | gbss (antisense) |
| | | | starch/carbohydrate | |
| Potato | HLMT15-15 | NA | Insect & disease res. | cry3A; pvy cp |
| Potato | HLMT15-3 | NA | Insect & disease res. | cry3A; pvy cp |
| Potato | HLMT15-46 | NA | Insect & disease res. | cry3A; pvy cp |
| Potato | RBMT15-101 | NMK-89653-6 | Insect & disease res. | cry3A; pvy cp |
| Potato | RBMT21-129 | NMK-89684-1 | Insect & disease res. | cry3A; plrv orf1; |
| | | | | plrv orf2 |
| Potato | RBMT21-152 | NA | Insect & disease res. | cry3A; plrv orf1; |
| | | | | plrv orf2 |
| Potato | RBMT21-350 | NMK-89185-6 | Insect & disease res. | cry3A; plrv orf1; |
| | | | | plrv orf2 |
| Potato | RBMT22-082 | NMK-89896-6 | Insect & disease res.; | cry3A; plrv orf1; |
| | | | Glyphosate tol. | plrv orf2; cp4 epsps |
| | | | | (aroA:CP4) |
| Potato | RBMT22-186 | NA | Insect & disease res.; | cry3A; plrv orf1; |
| | | | Glyphosate tol. | plrv orf2; cp4 epsps |
| | | | | (aroA:CP4) |
| Potato | RBMT22-238 | NA | Insect & disease res.; | cry3A; plrv orf1; |
| | | | Glyphosate tol. | plrv orf2; cp4 epsps |
| | | | | (aroA:CP4) |
| Potato | RBMT22-262 | NA | Insect & disease res.; | cry3A; plrv orf1; |
| | | | Glyphosate tol. | plrv orf2; cp4 epsps |
| | | | | (aroA:CP4) |
| Potato | SEMT15-02 | NMK-89935-9 | Insect & disease res. | cry3A; pvy cp |
| Potato | SEMT15-07 | NA | Insect & disease res. | cry3A; pvy cp |
| Potato | SEMT15-15 | NMK-89930-4 | Insect & disease res. | cry3A; pvy cp |
| Potato | SPBT02-5 | NMK-89576-1 | Insect res. | cry3A |
| Potato | SPBT02-7 | NMK-89724-5 | Insect res. | cry3A |
| Rice | 7Crp#242-95-7 | | Anti-allergy | 7crp |
| Rice | 7Crp#10 | NA | Anti-allergy | 7crp |
| Rice | GM Shanyou 63 | NA | Insect res. | cry1Ab; cry1Ac |
| Rice | Huahui-1/TT51-1 | NA | Insect res. | cry1Ab; cry1Ac |
| Rice | LLRICE06 | ACS-OS001-4 | Glufosinate tol. | bar |
| Rice | LLRICE601 | BCS-OS003-7 | Glufosinate tol. | bar |
| Rice | LLRICE62 | ACS-OS002-5 | Glufosinate tol. | bar |
| Rice | Tarom molaii + | NA | Insect res. | cry1Ab (truncated) |
| | cry1Ab | | | |
| Rice | GAT-OS2 | | Glufosinate tol. | bar |
| Rice | GAT-OS3 | | Glufosinate tol. | bar |
| Rice | PE-7 | | Insect res. | Cry1Ac |
| Rice | 7Crp#10 | NA | Anti-allergy | 7crp |
| Rice | KPD627-8 | | High tryptophan | OASA1D |
| Rice | KPD722-4 | | High tryptophan | OASA1D |
| Rice | KA317 | | High tryptophan | OASA1D |
| Rice | HW5 | | High tryptophan | OASA1D |
| Rice | HW1 | | High tryptophan | OASA1D |
| Rice | B-4-1-18 | | Erect leaves semidwarf | Δ OsBRI1 |
| Rice | G-3-3-22 | | Semidwarf | OSGA2ox1 |
| Rice | AD77 | | Disease res. | DEF |
| Rice | AD51 | | Disease res. | DEF |
| Rice | AD48 | | Disease res. | DEF |
| Rice | AD41 | | Disease res. | DEF |
| Rice | 13pNasNaatAprt1 | | Low iron tol. | HvNAS1; HvNAAT-A; |
| | | | | APRT |
| Rice | 13pAprt1 | | Low iron tol. | APRT |
| Rice | gHvNAS1- | | Low iron tol. | HvNAS1; HvNAAT-A; |
| | gHvNAAT-1 | | | HvNAAT-B |
| Rice | gHvIDS3-1 | | Low iron tol. | HvIDS3 |
| Rice | gHvNAAT1 | | Low iron tol. | HvNAAT-A; |
| | | | | HvNAAT-B |
| Rice | gHvNAS1-1 | | Low iron tol. | HvNAS1 |
| Rice | NIA-OS006-4 | | Disease res. | WRKY45 |
| Rice | NIA-OS005-3 | | Disease res. | WRKY45 |
| Rice | NIA-OS004-2 | | Disease res. | WRKY45 |
| Rice | NIA-OS003-1 | | Disease res. | WRKY45 |
| Rice | NIA-OS002-9 | | Disease res. | WRKY45 |
| Rice | NIA-OS001-8 | | Disease res. | WRKY45 |
| Rice | OsCr11 | | Anti-allergy | Modified Cry j |
| Rice | 17053 | | Glyphosate tol. | cp4 epsps (aroA:CP4) |
| Rice | 17314 | | Glyphosate tol. | cp4 epsps (aroA:CP4) |
| Rose | WKS82/130-4-1 | IFD-52401-4 | Modified flower color | 5AT; bp40 (f3′5′h) |
| Rose | WKS92/130-9-1 | IFD-52901-9 | Modified flower color | 5AT; bp40 (f3′5′h) |
| Soybean | 260-05 (G94-1, | NA | Modified oil/fatty acid | gm-fad2-1 |
| | G94-19, G168) | | | (silencing locus) |
| Soybean | A2704-12 | ACS-GM005-3 | Glufosinate tol. | pat |
| Soybean | A2704-21 | ACS-GM004-2 | Glufosinate tol. | pat |
| Soybean | A5547-127 | ACS-GM006-4 | Glufosinate tol. | pat |
| Soybean | A5547-35 | ACS-GM008-6 | Glufosinate tol. | pat |
| Soybean | CV127 | BPS-CV127-9 | Imidazolinone tol. | csr1-2 |
| Soybean | DAS68416-4 | DAS68416-4 | Glufosinate tol. | pat |
| Soybean | DP305423 | DP-305423-1 | Modified oil/fatty | gm-fad2-1 (silencing |
| | | | acid; ALS herbicide tol. | locus); gm-hra |
| Soybean | DP356043 | DP-356043-5 | Modified oil/fatty | gm-fad2-1 (silencing |
| | | | acid; glyphosate tol. | locus); gat4601 |
| Soybean | FG72 | MST-FG072-3 | Glyphosate & HPPD tol. | 2mepsps; hppdPF |
| | | | | W336 |
| Soybean | GTS 40-3-2 | MON-04032-6 | Glyphosate tol. | cp4 epsps (aroA:CP4) |
| | (40-3-2) | | | |
| Soybean | GU262 | ACS-GM003-1 | Glufosinate tol. | pat |
| Soybean | MON87701 | MON-87701-2 | Insect res. | cry1Ac |
| Soybean | MON87705 | MON-87705-6 | Modified oil/fatty | fatb1-A (sense & |
| | | | acid; glyphosate tol. | antisense); fad2-1A |
| | | | | (sense & antisense); |
| | | | | cp4 epsps (aroA:CP4) |
| Soybean | MON87708 | MON-87708-9 | Dicamba & glyphosate tol. | dmo; cp4 epsps |
| | | | | (aroA:CP4) |
| Soybean | MON87769 | MON-87769-7 | Modified oil/fatty | Pj.D6D; Nc.Fad3; cp4 |
| | | | acid; glyphosate tol. | epsps (aroA:CP4) |
| Soybean | MON89788 | MON-89788-1 | Glyphosate tol. | cp4 epsps (aroA:CP4) |
| Soybean | W62 | ACS-GM002-9 | Glufosinate tol. | bar |
| Soybean | W98 | ACS-GM001-8 | Glufosinate tol. | bar |
| Soybean | MON87754 | MON-87754-1 | High oil | dgat2A |
| Soybean | DAS21606 | DAS-21606 | Aryloxyalkanoate & | Modified aad-12; pat |
| | | | glufosinate tol. | |
| Soybean | DAS44406 | DAS-44406-6 | Aryloxyalkanoate, glyphosate | Modified aad-12; |
| | | | & glufosinate tol. | 2mepsps; pat |
| Soybean | SYHT04R | SYN-0004R-8 | Mesotrione tol. | Modified avhppd |
| Soybean | 9582.814.19.1 | | Insect res. & | cry1Ac, cry1F, PAT |
| | | | glufosinate tol. | |
| Squash | CZW3 | SEM-ØCZW3-2 | Disease res. | cmv cp, zymv cp, |
| | | | | wmv cp |
| Squash | ZW20 | SEM-0ZW20-7 | Disease res. | zymv cp, wmv cp |
| Sugar Beet | GTSB77 | SY-GTSB77-8 | Glyphosate tol. | cp4 epsps |
| | (T9100152) | | | (aroA:CP4); goxv247 |
| Sugar Beet | H7-1 | KM-000H71-4 | Glyphosate tol. | cp4 epsps (aroA:CP4) |
| Sugar Beet | T120-7 | ACS-BV001-3 | Glufosinate tol. | pat |
| Sugar Beet | T227-1 | | Glyphosate tol. | cp4 epsps (aroA:CP4) |
| Sugarcane | NXI-1T | | Drought tol. | EcbetA |
| Sunflower | X81359 | | Imidazolinone tol. | als |
| Sweet | PK-SP01 | NA | Disease res. | cmv cp |
| Pepper | | | | |
| Tobacco | C/F/93/08-02 | NA | Oxynil tol. | bxn |
| Tobacco | Vector 21-41 | NA | Reduced nicotine | NtQPT1 (antisense) |
| Tomato | 1345-4 | NA | Delayed | acc (truncated) |
| | | | ripening/senescense | |
| Tomato | 35-1-N | NA | Delayed | sam-k |
| | | | ripening/senescense | |
| Tomato | 5345 | NA | Insect res. | cry1Ac |
| Tomato | 8338 | CGN-89322-3 | Delayed | accd |
| | | | ripening/senescense | |
| Tomato | B | SYN-0000B-6 | Delayed | pg (sense or |
| | | | ripening/senescense | antisense) |
| Tomato | Da | SYN-0000DA-9 | Delayed | pg (sense or |
| | | | ripening/senescense | antisense) |
| Sunflower | X81359 | | Imidazolinone tol. | als |
| Tomato | Da Dong No 9 | NA | Modified product | NA |
| Tomato | F (1401F, h38F, | SYN-0000F-1 | Delayed | pg (sense or |
| | 11013F, 7913F) | | ripening/senescense | antisense) |
| Tomato | FLAVR SAVR ™ | CGN-89564-2 | Delayed | pg (sense or |
| | | | ripening/senescense | antisense) |
| Tomato | Huafan No 1 | NA | Delayed | anti-efe |
| | | | ripening/senescense | |
| Tomato | PK- ™8805R | NA | Disease res. | cmv cp |
| | (8805R) | | | |
| Wheat | MON71800 | MON-718ØØ-3 | Glyphosate tol. | cp4 epsps (aroA:CP4) |

*Argentine,
**Polish,
#Eggplant

Treatment of genetically modified plants and seeds with compounds of the disclosure may result in enhanced effects. For example, reduction in application rates, broadening of the activity spectrum, increased tolerance to biotic/abiotic stresses or enhanced storage stability may be greater than expected from just simple additive effects of the application of compounds of the disclosure on genetically modified plants and seeds.

Compounds of this disclosure are also useful in seed treatments for protecting seeds from invertebrate pests. In the context of the present disclosure and claims, treating a seed means contacting the seed with a biologically effective amount of a compound of this disclosure, which is typically formulated as a composition of the disclosure. This seed treatment protects the seed from invertebrate soil pests and generally can also protect roots and other plant parts in contact with the soil of the seedling developing from the germinating seed. The seed treatment may also provide protection of foliage by translocation of the compound of this disclosure or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this disclosure can also increase vigor of plants growing from the treated seed.

One method of seed treatment is by spraying or dusting the seed with a compound of the disclosure (i.e. as a formulated composition) before sowing the seeds. Compositions formulated for seed treatment generally comprise a film former or adhesive agent. Therefore typically a seed coating composition of the present disclosure comprises a biologically effective amount of a compound of Formula 1, an N-oxide or salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 and their compositions, both alone and in combination with other insecticides and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides with which compounds of Formula 1 can be formulated to provide mixtures useful in seed treatment include abamectin, acetamiprid, acrinathrin, amitraz, avermectin, azadirachtin, bensultap, bifenthrin, buprofezin, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of nuclear polyhedrosis viruses.

Fungicides with which compounds of Formula 1 can be formulated to provide mixtures useful in seed treatment include amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria such as Bacillus pumilus (e.g., strain GB34) and Bacillus firmus (e.g., isolate 1582), rhizobia inoculants/extenders, isoflavonoids and lipo-chitooligosaccharides.

The treated seed typically comprises a compound of the present disclosure in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this disclosure can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1, an N-oxide, or salt thereof, (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

The compounds of this disclosure can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present disclosure. Combinations with spray oils, spray oil concentrations, spreader stickers, adjuvants, other solvents, and piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present disclosure and a carrier. One embodiment of such a spray composition comprises a biologically effective amount of a compound or a composition of the present disclosure and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

One embodiment of the present disclosure relates to a method for controlling invertebrate pests, comprising diluting the pesticidal composition of the present disclosure (a compound of Formula 1 formulated with surfactants, solid diluents and liquid diluents or a formulated mixture of a compound of Formula 1 and at least one other pesticide) with water, and optionally adding an adjuvant to form a diluted composition, and contacting the invertebrate pest or its environment with an effective amount of said diluted composition.

Although a spray composition formed by diluting with water a sufficient concentration of the present pesticidal composition can provide sufficient efficacy for controlling invertebrate pests, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

Among the spray adjuvants, oils including crop oils, crop oil concentrates, vegetable oil concentrates and methylated seed oil concentrates are most commonly used to improve the efficacy of pesticides, possibly by means of promoting more even and uniform spray deposits. In situations where phytotoxicity potentially caused by oils or other water-immiscible liquids are of concern, spray compositions prepared from the composition of the present disclosure will generally not contain oil-based spray adjuvants. However, in situations where phytotoxicity caused by oil-based spray adjuvants is commercially insignificant, spray compositions prepared from the composition of the present composition can also contain oil-based spray adjuvants, which can potentially further increase control of invertebrate pests, as well as rainfastness.

Products identified as “crop oil” typically contain 95 to 98% paraffin or naphtha-based petroleum oil and 1 to 2% of one or more surfactants functioning as emulsifiers. Products identified as “crop oil concentrates” typically consist of 80 to 85% of emulsifiable petroleum-based oil and 15 to 20% of nonionic surfactants. Products correctly identified as “vegetable oil concentrates” typically consist of 80 to 85% of vegetable oil (i.e. seed or fruit oil, most commonly from cotton, linseed, soybean or sunflower) and 15 to 20% of nonionic surfactants. Adjuvant performance can be improved by replacing the vegetable oil with methyl esters of fatty acids that are typically derived from vegetable oils. Examples of methylated seed oil concentrates include MSO® Concentrate (UAP-Loveland Products, Inc.) and Premium MSO Methylated Spray Oil (Helena Chemical Company).

The amount of adjuvants added to spray mixtures generally does not exceed about 2.5% by volume, and more typically the amount is from about 0.1 to about 1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

Nonagronomic applications include protecting an animal, particularly a vertebrate, more particularly a homeothermic vertebrate (e.g., mammal or bird) and most particularly a mammal, from an invertebrate parasitic pest by administering a parasiticidally effective (i.e. biologically effective) amount of a compound of the disclosure, typically in the form of a composition formulated for veterinary use, to the animal to be protected. Therefore of note is a method for protecting an animal comprising administering to the animal a parasiticidally effective amount of a compound of the disclosure. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal. Examples of invertebrate parasitic pests controlled by administering a parasiticidally effective amount of a compound of the disclosure to an animal to be protected include ectoparasites (arthropods, acarines, etc) and endoparasites (helminths, e.g., nematodes, trematodes, cestodes, acanthocephalans, etc.). In particular, the compounds of this disclosure are effective against ectoparasites including: flies such as Haematobia (Lyperosia) irritans (horn fly), Stomoxys calcitrans (stable fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus instestinalis, Gastrophilus haemorrhoidalis and Gastrophilus naslis; lice such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; mites such as Psoroptes spp., Sarcoptes scabei, Chorioptes bovis, Demodex equi, Cheyletiella spp., Notoedres cati, Trombicula spp. and Otodectes cyanotis (ear mites); ticks such as Ixodes spp., Boophilus spp., Rhipicephalus spp., Amblyomma spp., Dermacentor spp., Hyalomma spp. and Haemaphysalis spp.; and fleas such as Ctenocephalides felis (cat flea) and Ctenocephalides canis (dog flea).

Nonagronomic applications in the veterinary sector are by conventional means such as by enteral administration in the form of, for example, tablets, capsules, drinks, drenching preparations, granulates, pastes, boli, feed-through procedures, or suppositories; or by parenteral administration, such as by injection (including intramuscular, subcutaneous, intravenous, intraperitoneal) or implants; by nasal administration; by topical administration, for example, in the form of immersion or dipping, spraying, washing, coating with powder, or application to a small area of the animal, and through articles such as neck collars, ear tags, tail bands, limb bands or halters which comprise compounds or compositions of the present disclosure.

Typically a parasiticidal composition according to the present disclosure comprises a mixture of a compound of Formula 1, an N-oxide or a salt thereof, with one or more pharmaceutically or veterinarily acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content. Therefore of note is a composition for protecting an animal from an invertebrate parasitic pest comprising a parasitically effective amount of a compound of the disclosure and at least one carrier.

For parenteral administration including intravenous, intramuscular and subcutaneous injection, a compound of the present disclosure can be formulated in suspension, solution or emulsion in oily or aqueous vehicles, and may contain adjuncts such as suspending, stabilizing and/or dispersing agents. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation.

For oral administration in the form of solutions (the most readily available form for absorption), emulsions, suspensions, pastes, gels, capsules, tablets, boluses powders, granules, rumen-retention and feed/water/lick blocks, a compound of the present disclosure can be formulated with binders/fillers known in the art to be suitable for oral administration compositions, such as sugars (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

If the parasiticidal compositions are in the form of feed concentrates, the carrier is typically selected from high-performance feed, feed cereals or protein concentrates. Such feed concentrate-containing compositions can, in addition to the parasiticidal active ingredients, comprise additives promoting animal health or growth, improving quality of meat from animals for slaughter or otherwise useful to animal husbandry. These additives can include, for example, vitamins, antibiotics, chemotherapeutics, bacteriostats, fungistats, coccidiostats and hormones.

Compounds of the present disclosure have been discovered to have favorable pharmacokinetic and pharmacodynamic properties providing systemic availability from oral administration and ingestion. Therefore after ingestion by the animal to be protected, parasiticidally effective concentrations of compounds of the disclosure in the bloodstream protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the disclosure, one or more carriers selected from binders and fillers suitable for oral administration and feed concentrate carriers).

Formulations for topical administration are typically in the form of a powder, cream, suspension, spray, emulsion, foam, paste, aerosol, ointment, salve or gel. More typically a topical formulation is a water-soluble solution, which can be in the form of a concentrate that is diluted before use. Parasiticidal compositions suitable for topical administration typically comprise a compound of the present disclosure and one or more topically suitable carriers. In applications of a parasiticidal composition topically to the exterior of an animal as a line or spot (i.e. “spot-on” treatment), the active ingredient migrates over the surface of the animal to cover most or all of its external surface area. As a result, the treated animal is particularly protected from invertebrate pests that feed off the epidermis of the animal such as ticks, fleas and lice. Therefore formulations for topical localized administration often comprise at least one organic solvent to facilitate transport of the active ingredient over the skin and/or penetration into the epidermis of the animal. Solvents commonly used as carriers in such formulations include propylene glycol, paraffins, aromatics, esters such as isopropyl myristate, glycol ethers, and alcohols such as ethanol and n-propanol.

The rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredients per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

In general for veterinary use, a compound of Formula 1, an N-oxide or a salt thereof, is administered in a parasiticidally effective amount to an animal to be protected from invertebrate parasite pests. A parasiticidally effective amount is the amount of active ingredient needed to achieve an observable effect diminishing the occurrence or activity of the target invertebrate parasite pest. One skilled in the art will appreciate that the parasitically effective dose can vary for the various compounds and compositions of the present disclosure, the desired parasitical effect and duration, the target invertebrate pest species, the animal to be protected, the mode of application and the like, and the amount needed to achieve a particular result can be determined through simple experimentation.

For oral administration to homeothermic animals, the daily dosage of a compound of the present disclosure typically ranges from about 0.01 mg/kg to about 100 mg/kg, more typically from about 0.5 mg/kg to about 100 mg/kg, of animal body weight. For topical (e.g., dermal) administration, dips and sprays typically contain from about 0.5 ppm to about 5000 ppm, more typically from about 1 ppm to about 3000 ppm, of a compound of the present disclosure.

The compounds of this disclosure prepared by the methods described herein are shown in Index Table A. Me means methyl, Et means ethyl and c-Pr means cyclo-propyl. The abbreviation “Cmpd. No.” stands for “Compound Number”, and the abbreviation “Ex.” stands for “Example” and is followed by a number indicating in which example the compound is prepared. The abbreviation “m.p.” stands for melting point. A wavy line or “-” in a structure fragment denotes the attachment point of the fragment to the remainder of the molecule. The numerical value reported in the column “MS” is the molecular weight of the highest isotopic abundance positively charged parent ion (M+1) formed by addition of H⁺ (molecular weight of 1) to the molecule having the highest isotopic abundance, or the highest isotopic abundance negatively charged ion (M-1) formed by loss of H⁺ (molecular weight of 1). The presence of molecular ions containing one or more higher atomic weight isotopes of lower abundance (e.g., ³⁷Cl, ⁸¹Br) is not reported. The reported MS peaks were observed by mass spectrometry using electrospray ionization (ESI) or atmospheric pressure chemical ionization (APCI). A dash “-” in the X column means that “X” is not present in the T substituent (e.g., T-9).

In Index Table A, J-1 through J-5 have the following meanings as defined below in Exhibit 5. As illustrated below the bond projecting to the left is connected at position J of group T.

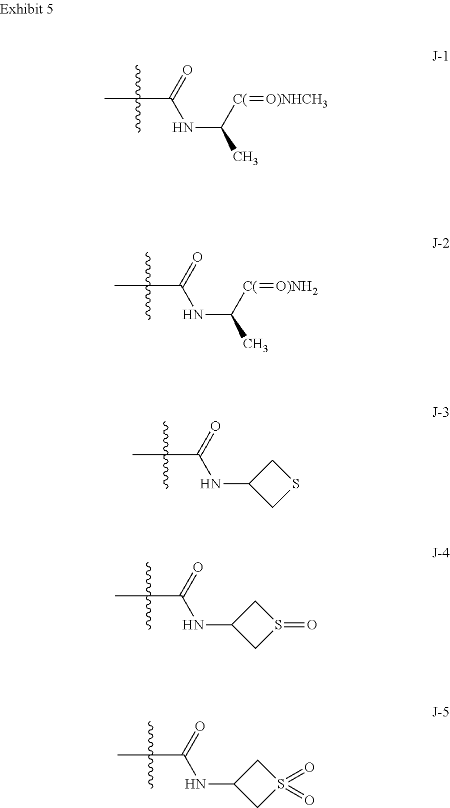

INDEX TABLE A

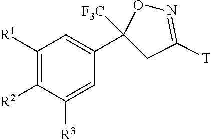

| Cmpd. No. | R¹ | R² | R³ | T | X | J | m.p. (° C.) | MS (M + 1) |
|---|---|---|---|---|---|---|---|---|
| 1 (Ex. 8) | CF₃ | H | H | T-1 | O | CH₂NHC(═O)Me | 155-158 | 484 |
| 2 | Cl | F | Cl | T-2 | O | C(═O)NHCH₂-c-Pr | 230-269 | 516 |
| 3 (Ex. 1) | Cl | F | Cl | T-2 | O | C(═O)NH-c-Pr | 180-238 | 502 |
| 4 | Cl | F | Cl | T-2 | O | C(═O)NH₂ | | 462 |
| 5 | Cl | H | Cl | T-6 | S | J-5 | 177-180 | |
| 6 | Cl | H | Cl | T-6 | S | J-4 | 245-248 | |
| 7 (Ex. 5) | Cl | H | Cl | T-6 | S | J-1 | 135-138 | |
| 8 | Cl | F | Cl | T-1 | O | C(═O)NH₂ | 208-211 | |
| 9 (Ex. 7) | CF₃ | H | H | T-1 | O | C(═O)NH₂ | 167-170 | |
| 10 | Cl | H | Cl | T-6 | S | J-3 | 206-209 | |
| 11 | Cl | H | Cl | T-6 | S | C(═O)NH-c-Pr | 181-184 | |
| 12 | Cl | H | Cl | T-6 | S | J-2 | 117-120 | |
| 13 | Cl | F | Cl | T-1 | O | C(═O)NH-c-Pr | 203-206 | |
| 14 | Cl | F | Cl | T-1 | O | C(═O)NHCH₂-c-Pr | 149-152 | |
| 15 (Ex. 6) | CF₃ | H | H | T-1 | O | C(═O)NH-c-Pr | 193-196 | |
| 16 | CF₃ | H | H | T-1 | O | C(═O)NHCH₂-c-Pr | 164-167 | 498 |
| 17 (Ex. 3) | Cl | F | Cl | T-4 | O | C(═O)NH-c-Pr | 195-218 | 504 |
| 18 | Cl | F | Cl | T-4 | O | C(═O)NH₂ | 209-244 | 464 |
| 19 | Cl | F | Cl | T-4 | O | J-3 | 195-223 | |
| 20 | Cl | F | Cl | T-5 | O | C(═O)NH₂ | 90-112 | 462 |
| 21 (Ex. 2) | Cl | F | Cl | T-5 | O | C(═O)NH-c-Pr | 159-167 | 502 |
| 22 | Cl | F | Cl | T-5 | O | C(═O)NHCH₂-c-Pr | 145-188 | 516 |
| 23 | CF₃ | H | H | T-1 | O | CH₂NHC(═O)Et | 150-153 | |
| 24 | Cl | H | Cl | T-2 | S | J-1 | 235-238 | |
| 25 | Cl | H | Cl | T-2 | S | J-2 | 241-244 | |
| 26 | Cl | H | Cl | T-2 | S | C(═O)NH-c-Pr | 180-183 | |
| 27 (Ex. 4) | Cl | H | Cl | T-2 | S | J-3 | 199-202 | |
| 28 | Cl | H | Cl | T-2 | S | J-4 | 155-158 | |
| 29 | Cl | H | Cl | T-2 | S | J-5 | 261-264 | |
| 30 | Cl | F | Cl | T-9 | — | J-1 | 135-138 | |
| 31 | Cl | F | Cl | T-9 | — | C(═O)NHCH₂-c-Pr | 109-112 | |
| 32 | Cl | F | Cl | T-9 | — | C(═O)NHCH₂—(pyrimidin-2-yl) | 159-161 | |

The following Tests demonstrate the control efficacy of compounds of this disclosure on specific pests. “Control efficacy” represents inhibition of invertebrate pest development (including mortality) that causes significantly reduced feeding. The pest control protection afforded by the compounds is not limited, however, to these species. See Index Table A for compound descriptions.

### BIOLOGICAL EXAMPLES

### Formulation and Spray Methodology for Tests A-G

Test compounds were formulated using a solution containing 10% acetone, 90% water and 300 ppm Activator 90® non-ionic surfactant (Loveland Products, Loveland, Colorado, USA). The formulated compounds were applied in 1 mL of liquid through an atomizer nozzle positioned 1.27 cm (0.5 inches) above the top of each test unit. Test compounds were sprayed at the rates indicated, and each test was replicated three times.

### Test A

For evaluating control of diamondback moth (Plutella xylostella (L.)) the test unit consisted of a small open container with a 12-14-day-old mustard plant inside. This was pre-infested with ˜50 neonate larvae that were dispensed into the test unit via corn cob grits using an inoculator. The larvae moved onto the test plant after being dispensed into the test unit.

Test compounds were formulated and sprayed at 2 and 0.4 ppm. After spraying of the formulated test compound, each test unit was allowed to dry for 1 hour and then a black, screened cap was placed on top. The test units were held for 6 days in a growth chamber at 25° C. and 70% relative humidity. Plant feeding damage was then visually assessed based on foliage consumed, and larvae were assessed for mortality.

Of the compounds of Formula 1 tested at 2 ppm, the following provided very good to excellent levels of control efficacy (40% or less feeding damage and/or 100% mortality): 1, 2, 4, 5, 6, 7, 8, 9, 10, 11, 12, 13, 14, 15, 16, 18, 23, 24, 25, 27, 28, 29, 30, 31, and 32.

Of the compounds of Formula 1 tested at 0.4 ppm, the following provided very good to excellent levels of control efficacy (40% or less feeding damage and/or 100% mortality): 1, 5, 6, 7, 10, 11, 12, 13, 14, 16, 23, 24, 28, 30, and 31.

### Test B

For evaluating control of fall armyworm (Spodoptera frugiperda (J. E. Smith)) the test unit consisted of a small open container with a 4-5-day-old corn (maize) plant inside. This was pre-infested with 10-15 1-day-old larvae on a piece of insect diet.

Test compounds were formulated and sprayed at 2 and 0.4 ppm. After spraying of the formulated test compound, the test units were maintained in a growth chamber for 6 days at 25° C. and 70% relative humidity. Plant feeding damage was then visually assessed based on foliage consumed, and larvae were assessed for mortality.

Of the compounds of Formula 1 tested at 2 ppm, the following provided very good to excellent levels of control efficacy (40% or less feeding damage and/or 100% mortality): 1, 5, 6, 7, 10, 11, 12, 13, 14, 15, 16, 23, 24, 27, 28, 29, 30, and 31.

Of the compounds of Formula 1 tested at 0.4 ppm, the following provided very good to excellent levels of control efficacy (40% or less feeding damage and/or 100% mortality): 1, 5, 7, 10, 13, 14, 23, and 30.

### Test C

For evaluating control of corn planthopper (Peregrinus maidis (Ashmead)) through contact and/or systemic means, the test unit consisted of a small open container with a 3-4-day-old corn (maize) plant inside. White sand was added to the top of the soil prior to application of the test compound.

Test compounds were formulated and sprayed at 50 and 10 ppm. After spraying of the formulated test compound, the test units were allowed to dry for 1 h before they were post-infested with ˜15-20 nymphs (18-to-21-day-old). A black, screened cap was placed on the top of each test unit, and the test units were held for 6 days in a growth chamber at 22-24° C. and 50-70% relative humidity. Each test unit was then visually assessed for insect mortality.

Of the compounds of Formula 1 tested at 50 ppm, the following resulted in at least 80% mortality: 1 and 23.

Of the compounds of Formula 1 tested at 10 ppm, the following resulted in at least 80% mortality: 23.

### Test D

For evaluating control of potato leafhopper (Empoascafabae (Harris)) through contact and/or systemic means, the test unit consisted of a small open container with a 5-6-day-old Soleil bean plant (primary leaves emerged) inside. White sand was added to the top of the soil, and one of the primary leaves was excised prior to application of the test compound.

Test compounds were formulated and sprayed at 50 and 10 ppm. After spraying of the formulated test compound, the test units were allowed to dry for 1 hour before they were post-infested with 5 potato leafhoppers (18-to-21-day-old adults). A black, screened cap was placed on the top of the test unit, and the test units were held for 6 days in a growth chamber at 20° C. and 70% relative humidity. Each test unit was then visually assessed for insect mortality.

Of the compounds of Formula 1 tested at 50 ppm, the following resulted in at least 80% mortality: 1, 6, 7, 8, 9, 10, 11, 12, 13, 14, 16, 23, 26, and 27.

Of the compounds of Formula 1 tested at 10 ppm, the following resulted in at least 80% mortality: 1, 6, 7, 11, 13, 14, 16, and 23.

### Test E

For evaluating control of green peach aphid (Myzus persicae (Sulzer)) through contact and/or systemic means, the test unit consisted of a small open container with a 12-15-day-old radish plant inside. This was pre-infested by placing on a leaf of the test plant 30-40 aphids on a piece of leaf excised from a culture plant (cut-leaf method). The aphids moved onto the test plant as the leaf piece desiccated. After pre-infestation, the soil of the test unit was covered with a layer of sand.

Test compounds were formulated and sprayed at 50 and 10 ppm. After spraying of the formulated test compound, each test unit was allowed to dry for 1 hour and then a black, screened cap was placed on top. The test units were held for 6 days in a growth chamber at 19-21° C. and 50-70% relative humidity. Each test unit was then visually assessed for insect mortality.

Of the compounds of Formula 1 tested at 50 ppm, the following resulted in at least 80% mortality: 1, 6, 7, 10, 13, and 23.

Of the compounds of Formula 1 tested at 10 ppm, the following resulted in at least 80% mortality: 10 and 23.

### Test F

For evaluating control of cotton melon aphid (Aphis gossypii (Glover)) through contact and/or systemic means, the test unit consisted of a small open container with a 5-day-old okra plant inside. This was pre-infested with 30-40 insects on a piece of leaf according to the cut-leaf method, and the soil of the test unit was covered with a layer of sand.

Test compounds were formulated and sprayed at 50 and 10 ppm. After spraying, the test units were maintained in a growth chamber for 6 days at 19° C. and 70% relative humidity. Each test unit was then visually assessed for insect mortality.

Of the compounds of Formula 1 tested at 50 ppm, the following resulted in at least 80% mortality: 1, 6, 7, 10, 12, 13, 23, 30, and 31.

Of the compounds of Formula 1 tested at 10 ppm, the following resulted in at least 80% mortality: 23.

### Test G

For evaluating control of the Western Flower Thrips (Frankliniellla occidentalis (Pergande)) through contact and/or systemic means, the test unit consisted of a small open container with an 8-9-day-old Soleil bean plant inside.

Test compounds were formulated and sprayed at 2 and 0.4 ppm. After spraying, the test units were allowed to dry for 1 hour, and then about 90 thrips (adults and nymphs) were added to each unit. A black, screened cap was placed on top, and the test units were held for 6 days at 25° C. and 45-55% relative humidity. Each test unit was then visually assessed for plant damage and insect mortality.

Of the compounds of Formula 1 tested at 2 ppm, the following provided very good to excellent levels of control efficacy (30% or less plant damage and/or 100% mortality): 5, 6, 7, 8, 10, 11, 12, 13, 14, 16, 23, 24, 28, and 30.

Of the compounds of Formula 1 tested at 0.4 ppm, the following provided very good to excellent levels of control efficacy (30% or less plant damage and/or 100% mortality): 6, 7, and 10.

What is claimed is:

1. A compound selected from Formula 1, an N-oxides or salt thereof, 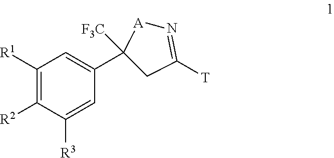
wherein
T is 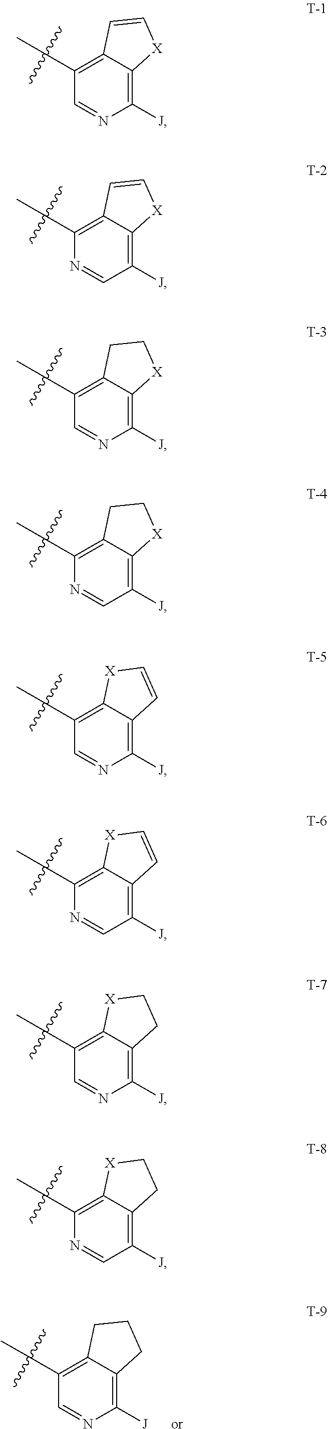 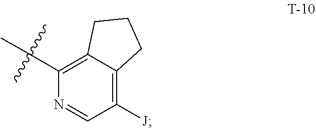
A is O, S or CH₂;
R¹, R², and R³ are each independently hydrogen, halogen, cyano, nitro, C₁-C₄ alkyl, C₁-C₄ haloalkyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
X is O or S;
J is C(═Z)NR⁴R⁵ or CH(R⁶)N(R¹³)C(═Z)R¹⁴;
each Z is independently O or S;
R⁴ is hydrogen, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl or C₂-C₇ alkoxycarbonyl;
R⁵ is hydrogen, OR¹⁰, NR¹¹R¹², SO₂NR¹¹R¹², C(R¹²)═NOR¹¹, CHR¹²NHR¹¹ or Q¹; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷; or
R⁴ and R⁵ are taken together with the nitrogen to which they are attached to form a 3- to 6-membered ring containing ring members selected from carbon atoms and up to two additional atoms independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
R⁶ is hydrogen, halogen, cyano, C₁-C₃ alkyl or C₁-C₃ haloalkyl;
each R⁷ is independently halogen, hydroxy, amino, cyano, nitro, Q², C₁-C₆ alkyl, C₁-C₆ alkoxy, C₁-C₆ alkylthio, C₁-C₆ alkylsulfinyl, C₁-C₆ alkylsulfonyl, C₁-C₆ alkylamino, C₂-C₈ dialkylamino, C₃-C₆ cycloalkylamino, C₂-C₇ alkylcarbonyl, C₂-C₇ alkoxycarbonyl, C₂-C₇ alkylaminocarbonyl, C₃-C₇ cycloalkylaminocarbonyl, C₃-C₇ alkenylaminocarbonyl, C₃-C₇ alkynylaminocarbonyl, C₃-C₉ dialkylaminocarbonyl, C₂-C₇ haloalkylcarbonyl, C₂-C₇ haloalkoxycarbonyl, C₂-C₇ haloalkylaminocarbonyl, C₃-C₉ halodialkylaminocarbonyl or —CONH₂;
Q¹ is a 5- or 6-membered aromatic ring or a 4- to 11-membered saturated or unsaturated ring or ring system, each optionally containing up to three heteroatoms selected from up to 1 oxygen, up to 1 sulfur and up to 3 nitrogen, wherein up to 2 carbon atom ring members are independently selected from C(═O) and C(═S) and the sulfur atom ring member is selected from S, S(═O) and S(═O)₂, each ring or ring system optionally substituted with one or more substituents independently selected from R⁸;
each Q² is independently a phenyl ring, a 5- or 6-membered aromatic heterocyclic ring or a 3- to 6-membered non-aromatic heterocyclic ring, each ring optionally substituted with one or more substituents independently selected from R⁹;
each R⁸ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, C₂-C₇ alkylaminocarbonyl, C₃-C₉ dialkylaminocarbonyl, —CONH₂, cyano or nitro;
each R⁹ is independently halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, cyano, nitro, phenyl or pyridinyl;
R¹⁰ is hydrogen; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more halogen;
R¹¹ is hydrogen, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl, C₂-C₇ haloalkylcarbonyl or C₂-C₇ alkoxycarbonyl;
R¹² is hydrogen or Q³; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷; or
R¹¹ and R¹² are taken together with the nitrogen to which they are attached to form a 3- to 6-membered ring containing ring members selected from carbon atoms and up to one additional atom independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
Q³ is a phenyl ring or a 5- or 6-membered heterocyclic ring, each ring optionally substituted with one or more substituents independently selected from R⁹;
R¹³ is hydrogen, C₁-C₆ alkyl, C₂-C₇ alkylcarbonyl, C₂-C₄ haloalkylcarbonyl, C₂-C₇ alkoxycarbonyl or C₂-C₄ alkoxyalkyl;
R¹⁴ is C₁-C₆ alkyl optionally substituted with halogen, OR¹⁹, S(═O)ₙR²⁰ or NR²¹C(═O)R²²; or
R¹⁴ is C₃-C₆ cycloalkyl or C₄-C₇ cycloalkylalkyl, each optionally substituted with up to one cyclopropyl and up to 4 substituents selected from halogen, cyano, C₁-C₂ alkyl and C₁-C₂ haloalkyl; or
R¹⁴ is (CH₂)ₘQ⁴; or
R¹⁴ is OR¹⁶ or NR^{17a}R^{17b};
Q⁴ is a 3- to 6-membered saturated heterocyclic ring containing ring members selected from carbon atoms and one heteroatom independently selected from one oxygen and one sulfur, wherein the sulfur atom ring member is selected from S, S(═O) or S(═O)₂, each ring optionally substituted with up to 2 substituents independently selected from R¹⁸;
R¹⁶ is C₁-C₄ alkyl or C₁-C₄ haloalkyl;
R^{17a} is hydrogen, C₁-C₄ alkyl, C₁-C₄ haloalkyl or C₃-C₆ cycloalkyl;
R^{17b} is hydrogen, C₁-C₄ alkyl, C₁-C₄ haloalkyl or C₃-C₆ cycloalkyl; or
R^{17a} and R^{17b} are taken together with the nitrogen to which they are attached to form a 3- to 6-membered ring containing ring members selected from carbon atoms and up to one additional atom independently selected from nitrogen, sulfur and oxygen, wherein the sulfur atom ring member is selected from S, S(═O) and S(═O)₂, said ring optionally substituted with 1 to 4 substituents independently selected from halogen, C₁-C₂ alkyl, C₁-C₂ haloalkyl, C₁-C₂ alkoxy, C₁-C₂ haloalkoxy, cyano and nitro;
each R¹⁸ is independently halogen, cyano, C₁-C₂ alkyl or C₁-C₂ haloalkyl;
R¹⁹ is hydrogen, C₁-C₄ alkyl or C₁-C₄ haloalkyl;
R²⁰ is C₁-C₄ alkyl or C₁-C₄ haloalkyl;
R²¹ is hydrogen or C₁-C₄ alkyl;
R²² is C₁-C₄ alkyl, C₁-C₄ haloalkyl or C₃-C₆ cycloalkyl;
n is independently 0, 1 or 2; and
m is 0 or 1.

2. A compound of claim 1 wherein:
A is O; and
R¹, R², and R³ are each independently hydrogen, halogen, cyano, C₁-C₄ alkyl, C₁-C₄ haloalkyl, C₁-C₄ alkoxy or C₁-C₄ haloalkoxy.

3. A compound of claim 2 wherein:
T is T-1, T-2, T-3, T-4, T-5, T-6 or T-9;
R¹, R², and R³ are each independently hydrogen, halogen, C₁-C₂ alkyl, C₁-C₂ haloalkyl, C₁-C₂ alkoxy or C₁-C₂ haloalkoxy; and
Z is O.

4. A compound of claim 3 wherein:
T is T-1, T-2, T-3, T-6 or T-9;
R¹, R², and R³ are each independently hydrogen, halogen or C₁-C₂ haloalkyl;
R⁴ is hydrogen, C₂-C₇ alkylcarbonyl or C₂-C₇ alkoxycarbonyl; and
R₆ is hydrogen, C₁-C₃ alkyl or C₁-C₃ haloalkyl.

5. A compound of claim 4 wherein:
R⁴ is hydrogen;
R⁵ is hydrogen or Q¹; or C₁-C₆ alkyl, C₃-C₆ cycloalkyl or C₄-C₇ cycloalkylalkyl, each optionally substituted with one or more substituents independently selected from R⁷;
R⁶ is hydrogen;
each R⁷ is independently C₁-C₄ alkyl, C₁-C₄ alkoxy, C₁-C₄ alkylthio, C₁-C₄ alkylsulfinyl, C₁-C₄ alkylsulfonyl, C₂-C₅ alkylcarbonyl, C₂-C₅ alkoxycarbonyl, C₂-C₅ alkylaminocarbonyl, C₃-C₅ cycloalkylaminocarbonyl or —CONH₂;
Q¹ is a 4- to 6-membered saturated or unsaturated ring, optionally containing up to two heteroatoms selected from up to 1 oxygen, up to 1 sulfur and up to 2 nitrogen, wherein up to 1 carbon atom ring member is C(═O) and the sulfur atom ring member is selected from S, S(═O) and S(═O)₂, each ring optionally substituted with one or more substituents independently selected from R⁸;
R¹³ is hydrogen, C₁-C₄ alkyl, C₂-C₇ alkylcarbonyl or C₂-C₇ alkoxycarbonyl; and
R¹⁴ is C₁-C₆ alkyl optionally substituted with halogen.

6. A compound of claim 5 wherein:
R⁵ is Q¹; or C₁-C₄ alkyl, cyclopropyl or cyclopropylmethyl, each optionally substituted with one or more substituents independently selected from R⁷;
Q¹ is a 4- to 6-membered saturated ring, optionally containing up to two heteroatoms selected from up to 1 oxygen, up to 1 sulfur and up to 2 nitrogen, wherein the sulfur atom ring member is selected from S, S(═O) and S(═O)₂;
R¹³ is hydrogen; and
R¹⁴ is methyl or ethyl.

7. A compound of claim 6 wherein:
T is T-1, T-2 or T-6.

8. The compound of claim 1 which is selected from the group:
N-(cyclopropylmethyl)-4-[5-(3,5-dichloro-4-fluorophenyl)-4,5-dihydro-5-(trifluoromethyl)-3-isoxazolyl]furo[2,3-c]pyridine-7-carboxamide;
N-cyclopropyl-4-[5-(3,5-dichloro-4-fluorophenyl)-4,5-dihydro-5-(trifluoro-methyl)-3-isoxazolyl]furo[2,3-c]pyridine-7-carboxamide;
N-cyclopropyl-7-[5-(3,5-dichlorophenyl)-4,5-dihydro-5-(trifluoromethyl)-3-isoxazolyl]thieno[2,3-c]pyridine-4-carboxamide;
7-[5-(3,5-dichlorophenyl)-4,5-dihydro-5-(trifluoromethyl)-3-isoxazolyl]-N-[(1R)-1-methyl-2-(methylamino)-2-oxoethyl]thieno[2,3-c]pyridine-4-carboxamide;
7-[5-(3,5-dichlorophenyl)-4,5-dihydro-5-(trifluoromethyl)-3-isoxazolyl]-N-(1,1-dioxido-3-thietanyl)thieno[2,3-c]pyridine-4-carboxamide;
N-[[4-[4,5-dihydro-5-(trifluoromethyl)-5-[3-(trifluoromethyl)phenyl]-3-isoxazolyl]furo[2,3-c]pyridin-7-yl]methyl]acetamid;
N-[[4-[4,5-dihydro-5-(trifluoromethyl)-5-[3-(trifluoromethyl)phenyl]-3-isoxazolyl]furo[2,3-c]pyridin-7-yl]methyl]propanamid;
4-[5-(3,5-dichlorophenyl)-4,5-dihydro-5-(trifluoromethyl)-3-isoxazolyl]-N-[(1R)-1-methyl-2-(methylamino)-2-oxoethyl]thieno[3,2-c]pyridine-7-carboxamide;
N-cyclopropyl-4-[5-(3,5-dichlorophenyl)-4,5-dihydro-5-(trifluoromethyl)-3-isoxazolyl]thieno[3,2-c]pyridine-7-carboxamide;
4-[5-(3,5-dichlorophenyl)-4,5-dihydro-5-(trifluoromethyl)-3-isoxazolyl]-N-(1,1-dioxido-3-thietanyl)thieno[3,2-c]pyridine-7-carboxamide;
N-[[4-[4,5-dihydro-5-(trifluoromethyl)-5-[3-(trifluoromethyl)phenyl]-3-isoxazolyl]-2,3-dihydrofuro[2,3-c]pyridin-7-yl]methyl]-propanamide;
4-[5-(3,5-dichloro-4-fluorophenyl)-4,5-dihydro-5-(trifluoromethyl)-3-isoxazolyl]-6,7-dihydro-N-[(1R)-1-methyl-2-(methylamino)-2-oxoethyl]-5H-cyclopenta[c]pyridine-1-carboxamide.

9. A composition comprising a compound of claim 1 and at least one additional component selected from the group consisting of surfactants, solid diluents and liquid diluents, said composition optionally further comprising at least one additional biologically active compound or agent.

10. The composition of claim 9 wherein the at least one additional biologically active compound or agent is selected from the group consisting of abamectin, acephate, acequinocyl, acetamiprid, acrinathrin, afidopyropen, amidoflumet, amitraz, avermectin, azadirachtin, azinphos-methyl, benfuracarb, bensultap, bifenthrin, bifenazate, bistrifluron, borate, buprofezin, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cyclaniliprole, cycloprothrin, cycloxaprid, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin, flonicamid, flubendiamide, flucythrinate, flufenerim, flufenoxuron, flufenoxystrobin, fluensulfone, fluopyram, flupiprole, flupyradifurone, fluvalinate, tau-fluvalinate, fonophos, formetanate, fosthiazate, halofenozide, heptafluthrin, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicotine, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, tetrachlorvinphos, tetramethrin, tetramethylfluthrin, thiacloprid, thiamethoxam, thiodicarb, thiosultap-sodium, tioxazafen, tolfenpyrad, tralomethrin, triazamate, trichlorfon, triflumezopyrim, triflumuron, Bacillus thuringiensis delta-endotoxins, all strains of Bacillus thuringiensis, all strains of nuclear polyhedrosis viruses entomopathogenic bacteria, entomopathogenic viruses and entomopathogenic fungi.

11. The composition of claim 10 wherein the at least one additional biologically active compound or agent is selected from the group consisting of abamectin, acetamiprid, acrinathrin, afidopyropen, amitraz, avermectin, azadirachtin, benfuracarb, bensultap, bifenthrin, buprofezin, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, fluensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nuclear polyhedrosis viruses.

12. A composition for protecting an animal from an invertebrate parasitic pest comprising a parasiticidally effective amount of a compound of claim 1 and at least one carrier.

13. The composition of claim 12 in a form for oral administration.

14. A method for controlling an invertebrate pest comprising contacting the invertebrate pest or its environment with a biologically effective amount of a compound of claim 1.

15. The method of claim 14 wherein the environment is a plant.

16. The method of claim 14 wherein the environment is an animal.

17. The method of claim 14 wherein the environment is a seed.

18. The method of claim 17 wherein the seed is coated with the compound of claim 1 formulated as a composition comprising a film former or adhesive agent.

19. A treated seed comprising a compound of claim 1 in an amount of from about 0.0001 to 1% by weight of the seed before treatment.
